(19) United States
(12) Patent Application Publication
(10) Pub. No.: US 2005/0215562 A1
(43) Pub. Date: Sep. 29, 2005

(54) Methods for treating protein aggregation disorders

(76) Inventors: Patrick Tremblay, Montreal (CA); Richard McLaughlin, Onalaska, WI (US)

Correspondence Address: LAHIVE & COCKFIELD, LLP., 28 STATE STREET, BOSTON, MA, 02109, US

(21) Appl. No.: 10/874,805

(22) Filed: Jun. 22, 2004

Related U.S. Application Data
(63) Continuation of application No. PCT/IB04/02405, filed on Jun. 21, 2004.
(60) Provisional application No. 60/480,918, filed on Jun. 23, 2003.
(60) Provisional application No. 60/512,017, filed on Oct. 17, 2003.

(51) Int. Cl. A61K 31/495; A61K 31/445; A61K 31/205

(52) U.S. Cl. 514/252.12; 514/317; 514/554

(57) ABSTRACT

The present invention is based, at least in part on the discovery of therapeutic agents capable of preventing, inhibiting or modulating abnormal processing, misfolding or aggregation of protein. The therapeutic agents of the invention may prevent, inhibit or modulate the formation of inclusions. The therapeutic agents of the invention may also be capable of facilitating clearance and/or blocking the cellular toxicity of inclusions to treat or ameliorate disorders characterized by protein aggregation. Compounds which bind to structural motifs commonly found in protein aggregates, such as β-sheets, would represent strong candidates for such compounds and are therefore desirable.

### RELATED APPLICATIONS

This application is a continuation of PCT/US2004/XXXX, filed Jun. 21, 2004 entitled Methods for Treating Protein Aggregation Disorders; and claims priority to U.S. provisional patent application No. 60/480,918, filed Jun. 23, 2003 and U.S. provisional application 60/512,017, filed Oct. 17, 2003, also entitled Methods for Treating Protein Aggregation Disorders.

This application is related to U.S. provisional patent application No. 60/480,984, filed Jun. 23, 2003, U.S. provisional patent application No. 60/512,116, filed Oct. 17, 2003, and U.S. application Ser. No. 10/______, filed Jun. 18, 2004, identified by Attorney Docket No. NBI-152, entitled Pharmaceutical Formulations of Amyloid-Inhibiting Compounds.

This application is related to U.S. provisional application 60/482,214, filed Jun. 23 2003, U.S. provisional application No. 60/436,379, filed Dec. 24, 2002, entitled Combination Therapy for the Treatment of Alzheimer's Disease, U.S. utility patent application Ser. No. 10/746,138, filed Dec. 24, 2003, International patent application no. PCT/CA2003/00201 1, identified by NBI-154PC, and U.S. application Ser. No. ______, filed Jun. 18, 2004, all entitled Therapeutic Formulations for the Treatment of Beta-Amyloid Related Diseases.

This application is related to U.S. provisional patent application No. 60/482,058, filed Jun. 23, 2003, U.S. provisional patent application No. 60/512,135, filed Oct. 17, 2003, both entitled Synthetic Process for Preparing Compounds for Treating Amyloidosis, and U.S. application Ser. No. 10/______, filed Jun. 18, 2004, identified by Attorney Docket No. NBI-156, entitled Improved Pharmaceutical Drug Candidates and Method for Preparation Thereof.

This application is also related to U.S. provisional patent application No. 60/480,906, filed Jun. 23, 2003, U.S. provisional patent application Ser. No. 60/512,047, filed Oct. 17, 2003, U.S. application Ser. No. 10/______, filed Jun. 18, 2004, identified by Attorney Docket No. NBI-162A, U.S. application Ser. No. 10/______, filed Jun. 18, 2004, identified by Attorney Docket No. NBI-162B, all entitled Methods and Compositions for Treating Amyloid-Related Diseases.

This application is also related to U.S. Provisional Patent Application Ser. No. 60/512,018, filed on Oct. 17, 2003, U.S. Provisional Patent Application Ser. No. 60/480,928, filed Jun. 23, 2003, and U.S. patent application Ser. No. 10/______, filed Jun. 18, 2004, identified by Attorney Docket No. NBI-163, all entitled Methods and Compositions for Treating Amyloid- and Epileptogenesis-Associated Diseases. This application is also related to Method for Treating Amyloidosis, U.S. patent application Ser. No. 08/463,548, now U.S. Pat. No. 5,972,328.

The entire contents of each of these patent applications are hereby expressly incorporated herein by reference including without limitation the specification, claims, and abstract, as well as any figures, tables, or drawings thereof.

### BACKGROUND

The biological function of a protein depends on its three dimensional structure, which is determined in large part by its amino acid sequence but also by the environment. In fact, protein conformation governs its interaction with other factors, which can participate in the regulation of protein function. The failure of polypeptides to adopt and maintain their proper structure through proper protein folding is a major threat to cell function and viability. Consequently, elaborate systems have evolved to protect cells from the deleterious effects of misfolded proteins.

The first line of defense against misfolded proteins is the molecular chaperones, which associate with nascent polypeptides as they emerge from the ribosome, promoting correct folding and preventing harmful interactions. Chaperones also assist in the refolding of proteins damaged by stress and cellular injuries (Netzer and Hartl. 1998. Trends Biochem Sci 23:68). Nonetheless, a large fraction of newly translated proteins fail to fold correctly, generating a substantial burden of defective polypeptide (Schubert, et al. 2000. Nature 404:770). These defective proteins are degraded primarily by the ubiquitin-proteasome system, a multi-component system that identifies and degrades unwanted proteins. Under some circumstances, misfolded proteins elude the quality control systems. When these misfolded proteins accumulate in sufficient quantities, they are prone to aggregation and become resistant to proteolysis. Insoluble protein aggregates (or precipitated proteins) may be deposited in microscopically visible inclusions (also referred to as bodies) or plaques, the characteristics of which are often indicative of disease and contain disease-specific proteins.

Proteasomes and ubiquitinated proteins accumulate when proteolysis is impaired and form organized aggregates or aggresomes. Once formed, protein oligomers and larger aggregates can harbor toxic properties (Wojcik and DeMartino. 2003. Int J Biochem Cell Biol 35:579; Bence, et al. 2001. Science 292:1552) by directly impairing critical cellular functions. Furthermore their accumulation within aggresomes or inclusions can impair the ubiquitin-proteasome system normally responsible for the elimination of such harmful misfolded proteins (see, Garcia-Mata, et al. 2002. Traffic 3:388). Since the chaperone and ubiquitin-dependent proteolysis systems are central to the regulation of such fundamental cellular events as cell division and apoptosis, blockage of this system may exacerbate the cellular toxicity resulting from the accumulation of the protein aggregates. However, some data indicate that the presence of inclusions, aggresomes or plaques may not be necessary for the toxic response observed in proteopathies (Klement, et al.1998. Cell 95:41). Nonetheless, the presence of inclusions, aggresomes or plaques is an excellent cellular marker for the characterization of cellular and in vivo mouse models which reconstitute human pathologies. Such markers can be used for screening as an indicator of the harmful protein aggregation events which lead to the formation of such inclusions.

Consequently, abnormalities of protein folding, oligomerization, aggregation or deposition may play an important role in the pathophysiology of a diverse set of chronically progressive degenerative disorders.

For example, a number of degenerative diseases are characterized by the presence of inclusions and plaques. Non-limiting examples of such diseases include the following: Parkinson's Disease (PD), diffuse Lewy body dementia (DLBD), multiple system atrophy (MSA), dystrophia myotonica, dentatorubro-pallidoluysian atrophy (DRPLA), Friedreich's ataxia, fragile X syndrome, fragile XE mental retardation, Machado-Joseph Disease, spinobulbar muscular atrophy (also known as Kennedy's Disease), spinocerebellar ataxia, Huntington's disease (HD), familial encephalopathy with neuroserpin inclusion bodies (FENIB), Pick's disease, corticobasal degeneration (CBD), progressive supranuclear palsy (PSP), amyotrophic lateral sclerosis/parkinsonism dementia complex, Amyotrophic Lateral Sclerosis (ALS), Down's syndrome, Age-Related Macular Degeneration, Cataract, and Wilson's Disease. In many cases, genetic mutations underlying the familial forms of these diseases have been identified. In most cases, however, the initiating idiopathic event facilitating or triggering the conformational transition of the target protein is unknown but ultimately results in the abnormal processing, misfolding, oligomerization or aggregation of protein, which triggers cellular toxicity. As part of a cellular detoxification, defense strategy or as an attempt to degrade the abnormally folded proteins, such abnormal proteins often accumulate in various types of cellular inclusions or aggresomes. These inclusions or aggresomes have thus become one of the pathological hallmarks of this diverse set of degenerative conditions. To date, such therapeutic agents and completely effective treatments for these diseases are not available.

### SUMMARY OF THE INVENTION

It would be desirable to have a therapeutic compound capable of preventing, inhibiting, or modulating abnormal processing, misfolding, or aggregation of proteins to prevent cellular damage and death. These therapeutic compounds would be useful in treating the diseases discussed in the above Background section and further treating those diseases described below. For example, compounds binding directly to the target protein and acting early in the protein oligomerization cascade couldinhibit the formation of aggregates, aggresomes or inclusions. Compounds capable of binding to the aggregates and blocking the cellular toxicity associated with these aggregates could also be effective at treating or ameliorating disorders caused by protein aggregation. Compounds that bind to structural features, such as β-sheets, fibril-like structures or hydrophobic domains commonly found in proteins which form such aggregates, would represent strong candidates for such therapeutic application and are therefore desirable. By preventing de novo formation of aggregates such compounds would be expected to facilitate the clearance and/or inhibit the toxicity of such abnormal proteins and of already formed protein aggregates.

The present invention is based, at least in part on the discovery of therapeutic agents capable of preventing, inhibiting or modulating abnormal processing, misfolding or aggregation of protein. The therapeutic agents of the invention may prevent, inhibit or modulate the formation of inclusions. The therapeutic agents of the invention may facilitate the clearance of protein aggregates. The therapeutic agents of the invention may also be capable of blocking the cellular toxicity of inclusions to treat or ameliorate disorders characterized by protein aggregation. The therapeutic agents of the invention may also be used to prevent or treat disorders of protein conformation or protein aggregation.

In one embodiment of the invention, compounds are provided which bind to target proteins that have a propensity to form β-sheet structures and thereby prevent, inhibit or modulate their misfolding, conformational transition, abnormal processing or aggregation In another embodiment, compounds bind to structural motifs commonly found in protein aggregates, such as β-sheets.

In another aspect of the invention, a method for screening for compounds that inhibit protein aggregation or treat Protein Aggregation Disorders is provided comprising screening for compounds binding to the protein whose aggregation characterizes the disorder, e.g., synuclein for PD.

In another embodiment, compounds are provided which prevent self-association, oligomerization or aggregation of such proteins, and the cellular toxicity associated with such events.

In another embodiment, compounds that bind to the target protein of interest prevent conformational transition toward β-sheet and the formation of oligomers, aggregates or fibrils that would naturally form following such changes.

In another embodiment, compounds are provided which, in cells cultured in vitro, prevent, inhibit or modulate the formation of aggresomes or inclusions that are indicative of the assembly of toxic protein oligomers, aggregates or fibrils.

In another embodiment, a method for screening for compounds for treating or preventing Protein Aggregation Disorder is provided, comprising administration of a compound to transgenic mice developing progressive degenerative changes modeling a human disease, and screening for compounds which prevent some or all of the degenerative changes normally associated with such condition. In further embodiments, the method may comprise determining the effectiveness of the test compound to facilitate clearance of the detrimental protein aggregates or determining the effectiveness of the test compound to facilitate the degradation of the detrimental protein aggregates.

In one embodiment of the invention, a method for treating or preventing a Protein Aggregation Disorder is provided, comprising administering one of the compounds of the invention to individuals having, or predisposed to such condition. In one embodiment, the Protein Aggregation Disorder is not an Amyloid Proteopathy.

In another embodiment of the invention, pharmaceutical compositions of compounds are provided comprising an effective amount of the compound to treat a Protein Aggregation Disorder and a pharmaceutically acceptable carrier. In one embodiment, the Protein Aggregation Disorder is not an Amyloid Proteopathy.

In one embodiment, a packaged composition for treatment of a Protein Aggregation Disorder is provided, with a compound with therapeutic activity, comprising a compound having a target therapeutic activity and directions for using and treating a Protein Aggregation Disorder.

In another embodiment, methods are provided that modulate detrimental protein aggregation comprising contacting a detrimental protein aggregate or a protein that has a propensity to form β-sheet structures with an effective amount of the compound of the invention such that detrimental protein aggregation is modulated, wherein said Protein Aggregation Disorder is not an Amyloid Proteopathy.

In another embodiment, methods are provided that modulate detrimental protein aggregation comprising contacting a detrimental protein aggregate or a protein that has a propensity to form β-sheet structures with an effective amount of the compound of the invention such that clearance of the detrimental protein aggregation is modulated, thereby modulating detrimental protein aggregation, wherein said Protein Aggregation Disorder is not an Amyloid Proteopathy.

In another embodiment, methods are provided that modulate detrimental protein aggregation comprising contacting a detrimental protein aggregate or a protein that has a propensity to form β-sheet structures with an effective amount of the compound of the invention such that cellular toxicity of the detrimental protein aggregation is modulated, thereby modulating detrimental protein aggregation, wherein said Protein Aggregation Disorder is not an Amyloid Proteopathy.

In one embodiment, a method is provided for treating or preventing a Neurofibrillary Tangle associated with tau in a subject comprising administering an effective amount of the compound of the invention such that the Neurofibrillary tangle associated with tau is treated or prevented.

In one embodiment, a method is provided for modulating a Neurofibrillary Tangle associated with tau in a subject comprising administering an effective amount of the compound of the invention such that the Neurofibrillary tangle associated with tau is modulated.

In one embodiment, a method is provided for treating or preventing an inclusion containing the α-synuclein NAC fragment in a subject comprising administering an effective amount of the compound of the invention such that the an inclusion containing the α-synuclein NAC fragment is treated or prevented.

In one embodiment, a method is provided for modulating an inclusion containing the a-synuclein NAC fragment in a subject comprising administering an effective amount of the compound of the invention such that the an inclusion containing the α-synuclein NAC fragment is modulated.

### BRIEF DESCRIPTION OF THE DRAWINGS

FIG. 1—A graph (computer generated) depicting the assembly of NAC peptide into β-sheets at various concentrations utilizing the Thioflavin T Assay as described in the Examples below.

FIG. 2—A circular dichroism analysis (computer generated) of the NAC peptide conformation as described in the Examples.

FIG. 3—Electron micrographs (computer generated) showing the appearance of NAC fibers as described in the Examples below.

FIG. 4—Electron micrographs (computer generated) showing the influence of heparin on NAC fiber formation as described in the Examples below.

FIG. 5—68 depict compounds of the invention, as described herein. The compounds depicted and their pharmaceutically acceptable salts, prodrugs (including esters and amides thereof), pharmaceutical compositions thereof, and the uses thereof in the methods described below are included as part of the invention.

### DETAILED DESCRIPTION OF THE INVENTION

The present invention provides methods and compounds that are useful in the prevention, treatment or modulation of Protein Aggregation Disorders. For convenience, some definitions of terms referred to herein are set forth below.

The biological function of a protein depends on its three dimensional structure, which is determined in large part by its amino acid sequence. As proteins emerge from the ribosome and begin the process of folding to form the appropriate three dimensional structures, they expose their hydrophobic domains, which may lead to unsuccessful associations and detrimental protein aggregation (Wetzel. 1994. Trends Biotechnol 12:193). As used herein, a “misfolded protein” is a protein or peptide that has not conformed to its appropriate three dimensional structure, often resulting in aggregation, oligomerization or fibrillization of the aberrant protein with itself or other proteins or peptides. As used herein, a “conformational change” is the event by which a normal protein undergoes a change which results in altered structural properties. Protein misfolding or conformational change may take place during the translation process or post-translationally. The event may occur due to, for example, particular mutations (familial or idiopathic), expanded polyglutamine repeats, DNA mutation or RNA modification, amino acid misincorporation, or unequal synthesis of subunits in a multisubunit protein or other protein modifications (Wetzel. 1994. Trends Biotechnol 12:193; Bonifacino, et al. 1989. J Cell Biol 109:173; Hurle, et al. 1994. Proc Natl Acad Sci USA, 91:5446; See Table, below). In particular, the absence (or reduction) of a natural binding partner in a multisubunit complex or the absence (or reduction) of a particular chaperone (see below) may result in the misfolding of a protein which normally interacts (directly or indirectly) with these proteins or factors. These changes may take place following post-translational modifications of the protein such as aberrant proteolytic processing, phosphorylation, methylation, acetylation, glycosylation, or nitrosylation of the target protein. Such modifications can be a consequence of particular mutations or of the activation of specific biochemical cellular pathways.

The following Table lists various types of post-translational protein modifications:

| No | Post-Translational Protein Modifications |
|---|---|
| 1 | pPoly(ADP-ribosyl)ation (Burkle, 2000) |
| 2 | Isoprenoids (Maltese, 1990) |
| 3 | Enzymatic Glycosylation (Guevara et al., 1998) (Berninsone & |
| | Hirschberg, 1998) |
| 4 | Acetylation (Ogryzko, 2001) |
| 5 | Methylation (Person et al., 2003) |
| 6 | S-Nitrosylation of protein Cys residues (Lane et al., 2001; |
| | Gu et al., 2002)) |
| 7 | Phosphorylation (Berninsone & Hirschberg, 1998) (Verkman & |
| | Mitra, 2000) |
| 8 | Mono-ADP-ribosylation (Okazaki & Moss, 1999) |
| 9 | Palmitoylation (Beers & Fisher, 1992) |
| 10 | Hydroxylation of prolyl residues (Rucker & Dubick, 1984) |
| 11 | Oxidative deamination of lysyl residues (Rucker & Dubick, 1984) |
| 12 | Crosslinks that covalently bond together polypeptide chains |
| | (Rucker & Dubick, 1984) |
| 13 | Tyrosine sulfation (Huttner, 1988) |
| 14 | Sulfation of glycans (Berninsone & Hirschberg, 1998) |
| 15 | Phosphorylation of glycans (Moses et al. 1997) |
| 16 | Carboxymethylation (Eggo et al., 1983) |
| 17 | Iodination (Eggo et al., 1983) |
| 18 | Tyrosinolation (Nath et al., 1981) |
| 19 | Oxidation of amino acid side chains, especially, side chains |
| | of cysteine, prolyl, arginyl, lysyl and histidinyl residues) (Nath |
| | et al., 1981) |
| 20 | Deamidation of asparaginyl and glutaminyl residues |
| | (Nath et al., 1981) |
| 21 | Addition of metal ions (Waggoner et al., 2000) |
| 22 | S-glutathionylation of Cys (Dalle-Donne et al., 2003) |
| 23 | Proteolysis (Nakayama et al., 2001) |
| 24 | Non-enzymatic reaction of sugars with amino groups of |
| | proteins (Munch et al., 1998) |
| 25 | Carboxylation of glutamine residues (Ware et al., 1989) |
| 26 | Proline and lysine hydroxylations (Uzawa et al., 1998) |
| 27 | Carboxy terminal amidation (Evans & Shine, 1991) |
| 28 | Citrullination (van Venrooij & Pruijn, 2000) |
| 29 | Carbamylation (Hasuike et al., 2002) |
| 30 | Sumoylation (Freiman and Tjian 2003) |

Beers, M. F. & Fisher, A. B. (1992). Am J Physiol 263, L151-60.
Berninsone, P. & Hirschberg, C. B. (1998).. Ann N Y Acad Sci 842, 91-9.
Burkle, A. (2000). Ann N Y Acad Sci 908, 126-32.
Dalle-Donne, I., Giustarini, D., Rossi, R., Colombo, R. & Milzani, A. (2003). Free Radic Biol Med 34, 23-32.
Eggo, M. C., Drucker, D., Cheifetz, R. & Burrow, G. N. (1983). Can J Biochem Cell Biol 61, 662-9.
Evans, H. F. & Shine, J. (1991). Endocrinology 129, 1682-4.
Freiman RN, Tjian R. (2003). Cell 112: 11-7
Gu, Z., Kaul, M., Yan, B., Kridel, S. J., Cui, J., Strongin, A., Smith, J. W., Liddington, R. C. & Lipton, S. A. (2002). Science 297, 1186-90.
Guevara, J., Espinosa, B., Zenteno, E., Vazguez, L., Luna, J., Perry, G. & Mena, R. (1998). J Neuropathol Exp Neurol 57, 905-14.
Hasuike, Y., Nakanishi, T., Maeda, K., Tanaka, T., Inoue, T. & Takamitsu, Y. (2002). Nephron 91, 228-34.
Huttner, W. B. (1988). Annu Rev Physiol 50, 363-76.
Lane, P., Hao, G. & Gross, S. S. (2001). Sci STKE 2001, RE1.
Maltese, W. A. (1990).. Faseb J 4, 3319-28.
Moses, J., Oldberg, A., Cheng, F. & Fransson, L. A. (1997). Eur J Biochem 248, 521-6.
Munch, G., Schinzel, R., Loske, C., Wong, A., Durany, N., Li, J. J., Vlassara, H., Smith, M. A., Perry, G. & Riederer, P. (1998). J Neural Transm 105, 439-61.
Nakayama, K. I., Hatakeyama, S. & Nakayama, K. (2001). Biochem Biophys Res Commun 282, 853-60.
Nath, J., Flavin, M. & Schiffmann, E. (1981). J Cell Biol 91, 232-9.
Ogryzko, V. V. (2001). Cell Mol Life Sci 58, 683-92.
Okazaki, I. J. & Moss, J. (1999). Annu Rev Nutr 19, 485-509.
Person, M. D., Monks, T. J. & Lau, S. S. (2003). Chem Res Toxicol 16, 598-608.
Rucker, R. B. & Dubick, M. A. (1984). Environ Health Perspect 55, 179-91.
Uzawa, K., Marshall, M. K., Katz, E. P., Tanzawa, H., Yeowell, H. N. & Yamauchi, M. (1998).. Biochem Biophys Res Commun 249, 652-5.
van Venrooij, W. J. & Pruijn, G. J. (2000). Arthritis Res 2, 249-51.
Verkman, A. S. & Mitra, A. K. (2000). Am J Physiol Renal Physiol 278, F13-28.
Waggoner, D. J., Drisaldi, B., Bartnikas, T. B., Casareno, R. L., Prohaska, J. R., Gitlin, J. D. & Harris, D. A. (2000). J Biol Chem 275, 7455-8.
Ware, J., Diuguid, D. L., Liebman, H. A., Rabiet, M. J., Kasper, C. K., Furie, B. C., Furie, B. & Stafford, D. W. (1989). J Biol Chem 264, 11401-6.

Alterations in post-translational modifications can be a consequence of particular mutations or of the activation of specific biochemical cellular pathways. The misfolding, oligomerization or aggregation can also occur as a result of a change in the cellular environment such as pH, temperature, ionic strength, redox environment or other stress imposed on a particular biological environment. For instance, pH and temperature changes result in the partial unfolding of proteins and trigger a stress response that mitigates cellular damage. Since a certain amount of misfolding is inevitable, cells have developed several systems to minimize such misfolding and dispose of misfolded proteins prior to aggregation (Wickner, et al. 1999. Science 286:1888).

The first line of defense against misfolded proteins is the molecular chaperones. As used herein, “molecular chaperones” or “chaperones” are molecules that associate with nascent polypeptides emerging from the ribosome, promoting correct folding and preventing harmful interactions. Chaperones also assist in the refolding of proteins damaged by stress and cellular injuries (Netzer and Hartl. 1998. Trends Biochem Sci 23:68). Chaperones bind to and stabilize exposed hydrophobic amino acid residues, and allow the protein to adopt and maintain a proper folding state by preventing incorrect intra- and intermolecular interactions between partially folded or unfolded polypeptides. In fact, many proteins require chaperones to fold properly (Hartl. 1996. Nature 381:571). Nonetheless, a large fraction of newly translated proteins fail to fold correctly, or undergo a post-translational conformational change, generating a substantial burden of defective polypeptide (Schubert, et al. 2000. Nature 404:770). A transient or permanent deficiency in a given chaperone can also result in the accumulation of misfolded proteins.

These defective proteins are degraded primarily by the ubiquitin-proteasome system. As used herein, the “ubiquitin-proteasome system” is a multi-component system that identifies and degrades unwanted proteins. As used herein, the “proteasome” is a multisubunit complex found in both the nucleus and cytosol. The proteasome mediates the degradation of cytosolic, nuclear (Hershko and Ciechanover. 1998. Ann Rev Biochem 67:425), secretory and transmembrane proteins (Hirsch and Ploegh. 2000. Trends Cell Biol 10:268). In addition to clearing defective proteins the ubiquitin-proteasome system also carries out selective degradation of short-lived normal proteins thereby contributing to the regulation of numerous cellular processes. Under some circumstances, misfolded proteins may evade the ubiquitin-proteasome surveillance systems designed to promote correct folding and eliminate faulty proteins. When these misfolded proteins accumulate in sufficient quantity, they are prone to aggregation and may become resistant to proteolysis. As used herein, “aggregates”, “inclusions”, “bodies”, “fibrils” and “plaques” are abnormal associations and accumulations of aberrant proteins that resist proteolysis and may or may not be associated with molecules of the proteasome system. In many cases these aggregates may colocalize with specific markers such as the cytoskeletal microtubular markers vimentin, β-tubulin and γ-tubulin and may be extracellula, intracellular, or nuclear.

Proteasomes and ubiquitinated proteins often accumulate when proteolysis is impaired and may form organized clusters within inclusions or plaques as part of the cellular detoxification or defense strategy. When these clusters are specifically delivered to inclusion bodies by dynein-dependent retrograde transport on microtubules and form in the pericentrosomal area those aggregates, as used herein, are termed “aggresomes”. The aggresomal pathway provides a mechanism by which aggregated proteins form particulate (approximately 200 nm) mini-aggregates that are transported on microtubules (MTs) towards the MT organizing center (MTOC) by a process mediated by the minus-end motor protein dynein. Once at the MTOC, the individual particles pack into a single, usually spherical aggresome (1-3 micron) that surrounds the MTOC. Aggresomes are dynamic: they recruit various chaperones and proteasomes, presumably to aid in the disposal of the aggregated proteins and to act as a cytoprotective mechanism to prevent cellular toxicity (Taylor, et al. 2003. Hum Mol Genet 12:749). In addition, the formation of an aggresome is likely to activate the autophagic clearance mechanism that terminates in lysosomal degradation. Hence, the aggresome pathway may provide a novel system to deliver aggregated proteins from the cytoplasm to lysosomes for degradation. Once formed, accumulating aggresomes (Wojcik and DeMartino. 2003. Int J Biochem Cell Biol 35:579) as well as protein oligomers or aggregates elsewhere in the cell (Bence, et al. 2001. Science 292:1552), may impair the function of the ubiquitin-proteasome system and become toxic. (For a review see, Garcia-Mata, et al. 2002 Traffic 3:388). Consequently, abnormalities of protein aggregation and deposition may play an important role in the pathophysiology of a diverse set of chronically progressive degenerative disorders.

In fact, studies have shown that many degenerative diseases are associated with the oligomerization, aggregation or fibrillization of various proteins (For a review see, Kakizuka. 1998. TIG 14:396). For example, a number of neurodegenerative disorders are caused by expanded CAG repeats encoding polyglutamine tracts. Proteins containing expanded polyglutamine repeats appear to self-aggregate and, as a result, cause neuronal cell death or degeneration. Non-limiting examples of these diseases are the following: spinobulbar muscular atrophy (SBMA) or Kennedy's disease, caused by expanded polyglutamine repeats in the gene encoding the androgen receptor (AR); Huntington's disease (HD), caused by expanded polyglutamine repeats in the huntingtin gene; spinocerebellar ataxia type 1 (SCA1) caused by increased polyglutamine repeats in the ataxin-1 gene; serpinopathies caused by mutations in serpin genes (serine protease inhibitors); spinocerebellar ataxia type 2 (SCA2) caused by increased polyglutamine repeats in the ataxin-3 gene; Machado-Joseph disease (MJD or SCA3) caused by increased polyglutamine repeats in the ataxin-3 gene; spinocerebellar ataxia type 6 (SCA6) caused by increased polyglutamine repeats in the ataxin-6 gene; spinocerebellar ataxia type 7 (SCA7) caused by increased polyglutamine repeats in the ataxin-7 gene; spinocerebellar ataxia type 17 (SCA17) caused by increased polyglutamine repeats in the ataxin-17 gene; dentatorubral-pallidolusian atrophy (DRPLA); and serpinopathies caused by mutations in the serpin genes. While each of these diseases can be caused by mutations in a distinct protein, they all share a common characteristic, namely aggregation through self-association. It has been demonstrated that polyglutamine proteins can undergo aggregation favored by lengthened polyglutamine-stretches, and that this aggregation induces or enhances cell death. Studies using transgenic mice also show that polyglutamine fragments are toxic and suggest that polyglutamine aggregation is central to neurodegeneration (Schilling, et al. 1999. Hum Mol Genet 8:397; Reddy, et al. 1998. Nat Gen 20:198; DiFiglia, et al. 1997. Science 277:1990; Yamamoto, et al. 2000. Cell 101:57).

Similarly, the major pathology of Frontotemporal Dementia and Parkinsonism linked to chromosome 17 (FTDP-17) is the assembly of tau proteins into filaments (called paired helical filaments or PHF) which form neurofibrillary tangles (NFT). This neuropathological feature is the characteristic change defining a family of conditions known as tauopathies which include Alzheimer's disease, Dementia pugilistica, Down syndrome, Prion diseases, Amyotrophic lateral sclerosis/parkinsonism-dementia complex, Argyophilic grain dementia, Corticobasal degeneration, Diffuse neurofibrillary tangles with calcification, Frontotemporal dementia/parkinsonism linked to chromosone-17, Hallervorden-Spatz disease, Multiple system atrophy (MSA), Nieman-Pick disease type C, Pick's disease, Progressive supranuclear palsy, Subacute sclerosing panencephalitis, and Tangle-predominant Alzheimer's disease (AD).

In Frontotemporal Dementia (FTD) aberrant assembly is caused by a post-translational modification, hyperphosphorylation, of the fully mature tau protein. Protein modifications can result from an upstream event such as that induced by overproduction of Aβ peptides in AD, or by the abnormal splicing events of the tau gene resulting from mutations in the intronic sequences of tau and associated with Frontotemporal Dementia (Spillantini, et al. 1998. Proc Natl Acad Sci USA 95:7737). Expression of mutated forms of tau in transgenic mice is sufficient to induce the development of neurofibrillary tangles resembling those characterized in human tauopathies, arguing that abnormal tau is sufficient to induce some forms of neurodegeneration. In transgenic mice expressing mutated forms of both the amyloid precursor protein (APP) gene and tau, mutant Tau proteins synergize with mutant APP to yield a disease with neuropathological changes resembling more closely those observed in AD (For a review see, Lee et al., 2001. Science 293:1446; Gotz; et al. 2001. Science 293:1491; Lewis, et al. 2001. Science 293:1487).

One of the pathological features of Parkinson's disease (PD) is the formation of cytoplasmic inclusion bodies named Lewy bodies; these are also found in Dementia with Lewy Bodies (DLBD) and Multiple System Atrophy (MSA). The major constituent of Lewy bodies is alpha-synuclein (α-synuclein). Thus, α-synuclein provides another example of a protein whose aggregation is linked to neurodegeneration. The accumulation of α-synuclein in Lewy bodies has been shown to be linked to some autosomal dominant mutations within the α-synuclein gene. These substitutions apparently favor conformational transitions within the mature protein that lead to its accumulation in pathologic oligomeric and protofibrillar forms (Polymeropoulos, et al. 1997. Science 276:2045; Kruger, et al. 1998. Nat Genet 18:106; Conway, et al. 2000. Proc Natl Acad Sci USA 97:571). PD is also linked to recessive mutations in a gene, Parkin (Kitada, et al. 1998. Nature 392:605; Lücking, et al. 2000. N Engl J Med 3421560; Ishikawa and Tsuji. 1996. Neurology 47160), whose activity as an E2 dependent ubiquitin protein ligase is important for the ubiquitination of proteins destined for degradation via the proteasome (Shimura, et al. 2000. Nat Genet 25:302). Therefore, the mutations which reduce or abrogate the function of Parkin result in the accumulation or aggregation of substrates that would otherwise betargeted for degradation. In the absence of Parkin activity, binding partners or substrates of Parkin, such as PaelR1, cdc-rel1, synphilin-1, α-synuclein, β- and γ-tubulin, some of which are directly toxic to cells (Petrucelli, et al. 2002. Neuron 36:1007; Imai, et al. 2001. Cell 105:891; Ren, et al. 2003. J Neurosci 23: 316) may accumulate and trigger cellular damage and death (For a review see, Cookson. 2003. Neuron 37:7). Another recent report indicates that dysregulation (mutations) in another α-synuclein interacting protein present in Lewy bodies, synphilin-1, may result in some cases of sporadic PD (Marx, et al. 2003. Hum Mol Genet 12:1223). Similarly, in Amyotrophic Lateral Sclerosis (ALS), inclusion bodies called hyaline inclusions are frequently observed and are known to contain precipitates of mutated superoxide dismutase (SOD1).proteins. Approximately 20% of familial ALS cases have also been linked to mutations in the SOD1 gene (Rosen, et al. 1993. Nature 362:59; Orrell, 2000. Neuromuscular Disord 10:63).

Altogether, these observations indicate that dysregulation of the proteosomal ubiquitin-mediated degradation pathways can lead to the accumulation of misfolded and aggregated proteins that form inclusions, underlying cellular toxicity and degeneration. Electron microscopic analysis indicates that these proteins typically contain granule-, filament- and fibril-like structures. These structures are similar, although distinct, to the amyloid structures that are observed in, for example, Alzheimer's disease and prion diseases. Insoluble “amyloid” aggregates display a characteristic red-green birefringence under polarized light after staining with Congo Red. While the aggregating proteins discussed above are not amyloid per se, they form a similar structure, including β-pleated sheet secondary structures. Hydrophobic regions are also characteristic of aggregating proteins. Thus, while many of the aggregating proteins found in Protein Aggregation Disorders are not amyloid per se, they share many of the structural characteristics of amyloid, namely β-sheets, fibril-like structures, and/or hydrophobic domains. Since all aggregates share common structural features, compounds which bind or inhibit amyloid formation by interacting with these structural motifs, e.g. β-sheets, may be effective at preventing or inhibiting protein aggregation involved in Protein Aggregation Disorders.

Screening for compounds which treat, modulate, prevent or inhibit detrimental protein aggregation therefore represents a rational and generic approach to the treatment or prevention of Protein Aggregation Disorders.

Generally, a “Protein Aggregation Disorder or Protein Aggregation Proteopathy” includes a disease, disorder or condition that is associated with detrimental protein aggregation in a subject. “Detrimental protein aggregation” is the undesirable and harmful accumulation, oligomerization, fibrillization or aggregation, of two or more, hetero- or homomeric, proteins or peptides. A detrimental protein aggregate may be deposited in bodies, inclusions or plaques, the characteristics of which are often indicative of disease and contain disease-specific proteins, e.g. α-synuclein-containing Lewy bodies in Parkinson's disease. A detrimental protein aggregate is a three dimensional structure that may contain, e.g., misfolded protein composed of β-sheets, fibril-like structures and/or highly hydrophobic domains that tend to aggregate and are toxic to cells. Furthermore, a detrimental protein aggregate may be described as amyloid-like, although it does not contain amyloid deposits and is not considered to be associated with an Amyloidosis as it does not adhere to the strict definition of amyloid, i.e., it does not display display red-green or apple-green birefringence under polarized light following staining with Congo red. As used herein, a “non-amyloid” detrimental protein aggregate or “proteopathy” is a detrimental protein aggregate that does not contain amyloid deposits. Non-limiting classes of Protein Aggregation Disorders or Proteopathies include Protein Conformational Disorders, Alpha-Synucleinopathies, Polyglutamine Diseases, Serpinopathies, Tauopathies or other related disorders. Non-limiting examples of Protein Aggregation Disorders include Parkinson's Disease (PD), diffuse Lewy body dementia (DLBD), multiple system atrophy (MSA), dystrophia myotonica, dentatorubro-pallidoluysian atrophy (DRPLA), Friedreich's ataxia, fragile X syndrome, fragile XE mental retardation, Machado-Joseph Disease (MJD or SCA3), spinobulbar muscular atrophy (also known as Kennedy's Disease), spinocerebellar ataxia type 1 (SCA1) gene, spinocerebellar ataxia type 2 (SCA2), spinocerebellar ataxia type 6 (SCA6), spinocerebellar ataxia type 7 (SCA7), spinocerebellar ataxia type 17 (SCA17), chronic liver diseases, Huntington's disease (HD), familial encephalopathy with neuroserpin inclusion bodies (FENIB), Pick's disease, corticobasal degeneration (CBD), progressive supranuclear palsy (PSP), amyotrophic lateral sclerosis/parkinsonism dementia complex, Amyotrophic Lateral Sclerosis (ALS), Cataract, serpinopathies, haemolytic anemia, cystic fibrosis, Wilson's Disease, neurofibromatosis type 2, demyelinating peripheral neuropathies, retinitis pigmentosa, Marfan syndrome, emphysema, idiopathic pulmonary fibrosis, Argyophilic grain dementia, corticobasal degeneration, diffuse neurofibrillary tangles with calcification, frontotemporal dementia/parkinsonism linked to chromosome 17, Hallervorden-Spatz disease, Nieman-Pick disease type C, or subacute sclerosing panencephalitis.

These classes and diseases will be discussed in further detail below. It should be understood that the invention includes an embodiment of Protein Aggregation Disorders that are associated with proteins having a structural features being selectively targeted by the compounds of the invention. Examples of such structural features include β-sheets, fibril-like structures and/or hydrophobic domains. It should also be understood that the Protein Aggregation Disorders of this invention are not intended to include Amyloid Proteopathies, or Amyloidosis or methods of modulating or inhibiting amyloid deposition. For specific examples, see below.

The effects of detrimental protein aggregation tend to be cumulative, with a progressive loss of cellular function that ultimately results in cellular death which underlies the diverse pathological conditions.

Amyloidosis

The pathological deposition of amyloid is characteristic of a group of diseases referred to as Amyloidosis, and includes AA (reactive) amyloidosis, AL amyloidoses, senile systemic amyloidosis, cerebral amyloidosis (including Alzheimer's disease and cerebral amyloid angiopathy), dialysis related amyloidosis, type II diabetes (caused by islet amyloid polypeptide, “IAPP”), and others, all of which are characterized by the presence of amyloid fibrils that have common morphologic properties, stain with specific dyes (e.g., Congo red), and have a characteristic red-green birefringent appearance in polarized light after staining. See, e.g., WO 2003/017994 A1. They also share common ultrastructural features and common X-ray diffraction and infrared spectra. The amyloidogenic proteins associated with each of these diseases, although different in amino acid sequence, have the similar properties of self-associating, forming oligomers and fibrils and binding to various other elements such as proteoglycan, amyloid P and/or complement component. Moreover, each amyloidogenic protein has amino acid sequences which, although different, show functional domain similarities with the ability to bind glycosaminoglycan (GAG) portion of proteoglycan (referred to as the GAG binding site) as well as regions which promote β-sheet formation. Many amyloidogenic proteins are formed by proteolytic cleavage of precursor proteins, e.g., serum amyloid A protein (“ApoSAA,” producing AA peptide), transthyretin (sometimes referred to as prealbumin), β-amyloid precursor protein (“βAPP,” producing Aβ peptide), and peptides derived from the N-terminal region of the kappa or lambda light chain of monoclonal immunoglobulin. See, e.g., 2001. Physiological Reviews Vol. 81.

Amyloid-related diseases can either be restricted to one organ or spread to several organs. The first instance is referred to as “localized amyloidosis” while the second is referred to as “systemic amyloidosis.”

Some amyloid diseases can be idiopathic, but most of these diseases appear as a complication of a previously existing disorder. For example, primary amyloidosis can appear without any other pathology or can follow plasma cell dyscrasia or multiple myeloma.

Secondary amyloidosis is usually seen associated with chronic infection (such as tuberculosis) or chronic inflammation (such as rheumatoid arthritis). A familial form of secondary amyloidosis is also seen in Familial Mediterranean Fever (FMF). This familial type of amyloidosis, as one of the other types of familial amyloidosis, is inherited and found in specific population groups. In both primary and secondary amyloidosis, deposits are found in several organs and are thus considered systemic amyloid diseases.

Another type of systemic amyloidosis is found in long-term hemodialysis patients. In each of these cases, a different amyloidogenic protein is involved in amyloid deposition.

“Localized amyloidoses” are those that tend to involve a single organ system. Different amyloids are also characterized by the type of protein present in the deposit. For example, neurodegenerative diseases such as scrapie, bovine spongiform encephalitis, Creutzfeldt-Jakob disease, and the like are characterized by the appearance and accumulation of a protease-resistant form of a prion protein, (referred to as PrP^{Sc} or PrP27-30) in the central nervous system. Similarly, Alzheimer's disease, another neurodegenerative disorder, is characterized by neuritic plaques and neurofibrillary tangles. In this case, the plaque and blood vessel amyloid is formed by the deposition of fibrillar Aβ amyloid protein. Other diseases such as adult-onset diabetes (type II diabetes) are characterized by the localized accumulation of amyloid in the pancreas.

As used herein, the terms “β-amyloid” or “amyloid-β” refer to amyloid-β proteins or peptides, amyloid β precursor proteins or peptides, intermediates, and modifications and fragments thereof, unless otherwise specifically indicated. In particular, “Aβ” refers to any peptide produced by proteolytic processing of the amyloid precursor protein (APP) gene product, especially peptides which are associated with amyloid pathologies, including Aβ₁₋₃₉, Aβ₁₋₄₀, Aβ₁₋₄₁, Aβ₁₋₄₂, and Aβ₁₋₄₃. As used herein, the terms “β amyloid,” “amyloid-β,” and “Aβ” are synonymous.

Unless otherwise specified, the term “amyloid” refers to amyloidogenic proteins, peptides, or fragments thereof which can be soluble (e.g., monomeric or oligomeric) or insoluble (e.g., having fibrillary structure or in amyloid plaque).

In an embodiment of the invention, a Protein Aggregation Disorder does not include Amyloid Proteopathies. As used herein, the term “Amyloid Proteopathy” means the following disorders (with the associated amyloidogenic protein in parentheses after the disease): reactive or secondary amyloidosis (AA); idiopathic (primary) amyloidosis; myeloma or macroglobulinemia-associated amyloidosis (amyloid λ L-chain or amyloid γ L-chain); familial amyloid polyneuropathy (Portuguese, Japanese, Swedish) (ATTR); familial amyloid cardiomyopathy [Danish] (ATTR); isolated cardiac amyloid (ATTR); systemic senile amyloidosis (ATTR); medullary carcinoma of the thyroid (procalcitonin); isolated atrial amyloid (atrial naturetic factor); familial amyloidosis [Finnish] (gelsolin); hereditary cerebral hemorrhage with amyloidosis [Icelandic] (cystatin C); familial amyloidotic polyneuropathy [Iowa ] (AApoA-I); accelerated senescence in mice (AApoA-II); fibrinogen-associated amyloid; lysozyme-associated amyloid; human prion diseases; transmissible spongiform encephalopathies; Scrapie (prion, or PrP); Creutzfeldt-Jacob disease (PrP); Gerstmann-Straussler-Scheinker syndrome (PrP); Fatal Familial Insomnia (PrP); bovine spongiforn encephalitis (PrP); Alzheimer's disease (Aβ); Cerebral Amyloid Angiopathy (Aβ); Hereditary cerebral hemorrhage (Aβ); fainilial Mediterranean Fever; familial amyloid nephropathy with urticaria and deafness; Muckle-Wells syndrome; dialysis-related amyloidosis (β2-microglobulin); type II diabetes (IAPP); Down's syndrome (Aβ); Age-Related Macular Degeneration (Aβ); and Inclusion Body Myositis (Aβ). Amyloid. proteopathies may be familial or idiopathic or sporadic or infectious, and all forms of the diseases listed above are meant to be included. It should be understood that the term Amyloid Proteopathy is intended to include the disorders described in WO 96/28287, published. Sep. 19, 1996, WO 00/64420, published Nov. 2, 2000, and WO 94/22437, published Oct. 13, 1994.

Protein Conformational Disorders

A group of diverse diseases have been grouped together under the name of protein conformational disorders (PCDs) (Carrell and Lomas. 1997. Lancet 350:134; Kelly. 1996. Curr Opin Struct Biol 6:11; Thomas, et al. 1995. Trends Biochem Sci 20:456; Soto. 1999. J Mol Med 77:412; Carrell and Gooptu. 1998. Curr Opin Struct. Biol 8:799). Non-limiting examples of this group include serpinopathies, haemolytic anemia, Huntington's Disease (HD), cystic fibrosis, amyotrophic lateral sclerosis (ALS), and Parkinson disease (PD). It also includes amyloid-related diseases such as, for example, Alzheimer's disease (AD), transmissible spongiform encephalopathies (TSEs), Diabetes Type II, dialysis-related amyloidosis, secondary (AA) amyloidosis, cerebral amyloid angiopathy, inclusion body myositis, Down's syndrome and Age-Related Macular Degeneration. PCDs also include spinobulbar muscular atrophy (SBMA) or Kennedy's disease, Huntington's disease (HD), spinocerebellar ataxia type 1 (SCA1); spinocerebellar ataxia type 2 (SCA2), Machado-Joseph disease (MJD or SCA3), spinocerebellar ataxia type 6 (SCA6), spinocerebellar ataxia type 7 (SCA7), spinocerebellar ataxia type 17 (SCA17), dentatorubral-pallidolusian atrophy (DRPLA), dystrophia myotonica, Pick's Disease, corticobasal degeneration, progressive supranuclear palsy, amyotrophic lateral sclerosis/parkinsonism dementia complex, Friedreich's ataxia, fragile XE mental retardation, fragile X syndrome, Wilson's Disease, chronic liver diseases, and cataracts.

The defining characteristic in PCD is a change in the secondary or tertiary structure of a normal protein. The conformational change may promote the disease by either gain of a toxic activity or by the lack of biologicalfunction of the natively folded protein (Thomas, et al. 1995. Trends Biochem Sci 20:456; Carrell and Gooptu. 1998. Curr Opin Struct Biol 8:799). There is no evident sequence or structural homology among the proteins implicated in PCD, however, the striking feature of these proteins is their inherent ability to adopt at least two different stable conformations (Carrell and Gooptu. 1998. Curr Opin Struct Biol 8:799). In most PCDs the misfolded protein is rich in β-sheet conformation (Soto. 1999. J Mol Med 77:412; Carrell and Gooptu. 1998. Curr Opin Struct Biol 8:799). β-Sheets are one of the prevalent, repetitive secondary structures in folded proteins and are formed of alternating peptide-pleated strands linked by hydrogen bonding between the NH and CO groups of the peptide bond. While in α-helices the hydrogen bonds are between groups within the same strand, in β-sheets the bonds are between one strand and another. Since the second β-strand can come from a different region of the same protein or from a different molecule, formation of β-sheets is usually stabilized by protein oligomerization or aggregation. Indeed, in most PCDs the misfolded protein self-associates and becomes deposited in various types of aggregates or inclusions in diverse organs, inducing tissue damage and organ dysfunction (Kelly. 1996 Curr Opin Struct Biol 6:11). In one embodiment of the invention, a Protein Conformational Disorder does not include Amyloid Proteopathies.

Alpha-Synucleinopathies

Generally, the Alpha-Synucleinopathies include Parkinson's disease (PD) and other related disorders including diffuse Lewy body dementia (DLBD; also known as Lewy body disease), Shy-Drager syndrome, Neurologic orthostatic hypotension, Shy-McGee-Drager syndrome, Parkinson's plus syndrome and multiple system atrophy (MSA; a name grouping the four cerebral degenerative diseases of Shy-Drager syndrome, Neurologic orthostatic hypotension, and Parkinson's plus syndrome). The commonality of this group of disorders is the abnormal deposition of alpha-synuclein (α-synuclein) in the cytoplasm of neurons or glial cells forming inclusions referred to as Lewy bodies.

In Parkinson's disease and diffuse Lewy body dementia, α-synuclein is the main component of Lewy bodies and dystrophic neurites; α-synuclein also accumulates in the cytoplasm of glial cells. In multiple system atrophy, α-synuclein forms cytoplasmic oligodendroglial inclusions and neuronal inclusions that are the hallmark of this disease.

Alpha-synuclein is a protein of 140 amino acids. Its function is unknown, but it has been shown to have chaperone activity (Souza, et al. 2000. FEBS Lett 474:116) and it has been proposed that it functions in regulating synaptic vesicle formation (Murphy, et al. 2000. J Neurosci 29:3214). Furthermore, α-synuclein has been shown to bind to tau (Jensen, et al. 1999. J Biol Chem 274:25481) and microtubule-associated protein, MAP-1B (Jensen, et al. 2000. J Biol Chem 275:21500).

Accumulations of α-synuclein in the α-synucleinopathies have in common a fibrillar configuration, giving rise to insoluble forms and high molecular weight aggregates in vitro, but they differ in the binding of α-synuclein to distinct proteins with the exception of ubiquitin whose binding to α-synuclein is common to all α-synuclein inclusions (For a review see, Ferrer. 2001. Neurologia 16:163). For example, the Lewy bodies in patients with MSA have been found to contain 14-3-3 proteins, which mediate several types of signal transduction pathways (Kawamoto, et al. 2002. Ann Neurol 52:722); patients with PD have Lewy bodies that contain Synphilin-1 associated with α-synuclein (Wakabayashi, et al. 2000. Ann Neurol 47:521) and in patients with DLBD, cyclin-dependent kinase 5 (Cdk5) has been found in Lewy bodies Takahashi, et al. 2000. Brain Res 862:253).

Transgenic mice expressing alpha-synuclein mutants A30P and A53T have been developed. These mice develop early onset progressive decline of motor function. Neuropathologically these animals display typical α-synuclein immunoreactive Lewy body inclusions in the neurites (Putten, et al. 2000. J Neurosci 20:6021; Sommer, et al. 2000. Exp Gerontol 35:1389; Kahle, et al. 2002. J Clin. Invest 110:1429; Kahle, et al. 2001. Am J Pathol. 159:2215; Gomez-Isla, et al. 2003. Neurobiology Aging 24: 245-258; Lee, et al. 2002. Proc Natl Acad Sci USA 99:8968).

Parkinson's Disease

Parkinson's Disease (PD) is a slowly progressive late-onset neurodegenerative disorder. It is characterized by muscular rigidity, postural instability and trembling.

Recent data has implicated several genetic factors in the etiology of PD. Familial aggregation studies suggest that late-onset PD has a significant genetic etiology (Payami, et al. 2002. Arch Neurol 59:848). Heritable forms of PD are caused by gene mutations. To date, four genes (β-synuclein, parkin, COOH-terminal hydrolase L1 and DJ-1) and several additional loci have been shown to be associated with familial forms of PD (For a review see, Shastry. 2000. Neuroscientist 6:234; Shasrty. 2001. Neurosci Res 41:5; Cookson. 2003. Neuron 37:7). Autosomal dominant PD is due to mutations in the α-synuclein gene and autosomal recessive PD is due to mutations in the parkin gene.

Several lines of evidence suggest that in all known forms of PD, detrimental protein aggregation in dopaminergic neurons of the substantia nigra is the common mechanism of neurodegeneration. Three proteins whose mutations are associated with development of PD (α-synuclein, parkin and COOH-terminal hydrolase L1) are also present in Lewy bodies in sporadic PD (Mouradian. 2002. Neurology 58:179) and in DLBL (Schlossmacher, et al. 2002. Am J Pathol 160:1655). One of the pathological features of Parkinson's disease is the formation of cytoplasmic inclusion bodies named Lewy bodies; these are also found in DLBD and MSA. The major constituent of Lewy bodies is α-synuclein. Thus, α-synuclein provides another example of a protein whose aggregation is linked to neurodegeneration. The accumulation of α-synuclein in Lewy bodies has been shown to be linked to some autosomal dominant mutations within the α-synuclein gene. These substitutions apparently favor conformational transitions within the mature protein that leads to its accumulation in a pathologic oligomeric and protofibrillar form (Polymeropoulos, et al. 1997. Science 276:2045; Kruger, et al. 1998. Nat Genet 18:106; Conway, et al. 2000. Proc Natl Acad Sci USA 97:571). PD is also linked to recessive mutations in a gene, Parkin (Kitada, et al. 1998. Nature 392:605; Lücking, et al. 2000. N Engl J Med 342:1560; Ishikawa and Tsuji. 1996. Neurology 47:160), whose activity as an E2 dependent ubiquitin protein ligase is important for the ubiquitination of proteins destined to degradation via the proteasome (Shimura, et al. 2000. Nat Genet 25:302). Therefore, the loss of function of Parkin results in the accumulation or aggregation of substrates that would otherwise be targeted for degradation. In the absence of Parkin activity, binding partners or substrates of Parkin such as PaelR1, cdc-rel1, synphilin-1, α-synuclein, α- and γ-tubulin, some of which are directly toxic to cells (Petrucelli, et al. 2002. Neuron 36:1007; Imai, et al. 2001. Cell 105:891; Ren, et al. 2003. J Neurosc 23:3316), may accumulate and trigger cellular damage and death (For a review see, Cookson. 2003. Neuron 37:7). Another recent report indicates that dysregulation (mutations) in another α-synuclein interacting protein present in Lewy bodies, synphilin-1, may result in some cases of sporadic PD (Marx, et al. 2003. Hum Mol Genet 12:1223). Altogether, these observations indicate that dysregulation of the proteosomal ubiquitin-mediated degradation pathways can lead to the accumulation of misfolded and aggregated proteins that form inclusions underlying cellular toxicity and degeneration.

Importantly, accumulation of α-synuclein in cultured human cells selectively degenerates dopaminergic neurons in the presence of dopamine but not non-dopamine neurons, suggesting selective toxicity of its accumulation (Xu, et al. 2002. Nat Med, 8, 600). Recombinant α-synuclein incubated at 37° C. for long periods forms aggregates and fibrils, in vitro, with a morphology similar to those fibrils isolated from Lewy bodies (El-Agnaf, et al. 1998. FEBS Lett 440:67; Conway, et al. 1998. Nat Med 4:1318). Overexpression of Parkin in the presence of proteasome inhibitors results in the formation of aggresome-like inclusions (Junn, et al. 2002. J Biol Chem 6:47870). Furthermore, mice expressing known human α-synuclein mutations exhibit adult-onset neurodegeneration and the aggregation of α-synuclein in the brain (Lee, et al. 2002. Proc Natl Acad Sci USA, 99, 8968; Kahle, et al. 2001. Am J Pathol. 159:2215; Kahle, et al. 2002. J Clin Invest 110:1429; Gomez-Isla, et al 2003. Neurobiology Aging 24:245).

Recent findings have demonstrated that a high percentage of Alzheimer's Disease (AD) patients also form Lewy bodies, most abundantly in the amygdala (Hamilton. 2000. Brain Pathol, 10:378; Mukaetova-Ladinska, et al. 2000. J Neuropathol Exp Neurol 59:408). Interestingly, the highly hydrophobic non-amyloid component (NAC) region of α-synuclein has also been described as the second most abundant component of amyloid plaques in the brain of AD patients, after Aβ.

Alpha-synuclein has been shown to form fibrils in vitro. Futhermore it binds to Aβ and promotes its aggregation (Yoshimoto, et al. 1995. Proc Natl Acad Sci USA 92:9141). It was in fact originally identified as the precursor of the non-amyloid beta (Aβ) component (NAD) of AD plaques (Ueda, et al. 1993. Proc Natl Acad Sci USA 90:11282; Iwai. 2000. Biochem Biophys Acta 1502:95;Masliah, et al. 1996. Am J Pathol 148:201). NAC is a 35 amino acid long peptide with highly hydrophobic stretches which can self-aggregate and form fibrils in vitro. Moreover, these fibrils can efficiently seed the formation of Aβ fibrils in vitro (Han, et al. 1995. Chem Biol. 2: 163-169; Iwai, et al. 1995. Biochemistry 34:10139). It is in fact through its NAC domain that alpha-synuclein retains its fibrillogenic properties. Modulating the properties of NAC or targeting NAC with the compounds of the invention could therefore be a valid therapeutic avenue to inhibit the formation of protein aggregates and inclusions associated with alpha-synucleopathies, as well as the formation of aggregates between the beta-amyloid peptide and NAC of alpha-synuclein.

Polylutamine Disorders

Several adult onset diseases with progressive degeneration of the nervous system that is typically fatal have been shown to be caused by the expansion of [CAG]n (poyglutamine or polyQ) tracts in specific target proteins. To date, these diseases include dystrophia myotonica, dentatorubro-pallidoluysian atrophy (DRPLA), Friedreich's ataxia, fragile X syndrome, fragile XE mental retardation, Machado-Joseph disease, spinobulbar muscular atrophy (also known as Kennedy's Disease), spinocerebellar ataxia and Huntington's disease (HD) (Kaneko, et al. 1997. Proc Natl Acad Sci 94:10069; Zoghbi and Orr. 2000. Ann Rev Neurosci, 23:217).

The prototypical polyglutamine disease, Huntington's disease (HD), is an autosomal dominant neurodegenerative disorder characterized by involuntary movements, cognitive impairment progressing to dementia, and mood disturbances. The disorder is characterized by the selective loss of striatal neurons caused by expansion of poyglutamine tracts in the huntingtin (HD) gene (Shastry. 1994. Nasir, et al. 1996; Tobin and Singer. 2000. Trends Cell Biol, 10,531). The mutated proteins (or polyglutamine-containing subfragments) form ubiquitinated aggregates in neurons of patients or mouse models, in most cases within the nucleus.

Although the genes responsible for the polyQ diseases appear to be functionally unrelated, they all share the common feature of a CAG trinucleotide repeat in each gene's coding region that results in a polyglutamnine tract in the protein. In the normal population, the length of the polyQ repeat generally ranges from 10-36 consecutive glutamine residues. In each of these disease states, however, expansion of the poyQ tract beyond the normal range results in adult-onset slowly progressive neurodegeneration. Longer expansions correlate with earlier onset and more severe disease.

These diseases likely share a common molecular pathogenesis resulting from toxicity with the expanded polyglutamine tract. It is now clear that expanded polyQ endows the disease proteins with a dominant gain of function that is toxic to neurons. Each of the polyQ disease is characterized by a different pattern of neurodegeneration and thus different clinical manifestations. The selective vulnerability of different populations of neurons in these diseases is poorly understood, but it is likely related to the expression pattern of each disease gene and the normal function and interactions of the disease gene product (Dragatsis, et al. 2000. Nature Genet 26:300; Zuccato, et al. 2001. Science 293:493).

It has been recognized that expanded polyQ forms neuronal intranuclear inclusions in animal models of the polyQ diseases and the central nervous system of patients with the diseases (Ross. 1997. Neuron 19:1147). These inclusions consist of accumulations of insoluble aggregated polyQ-containing proteins or polyQ-containing fragments in association with other proteins. It has been proposed that proteins with long polyQ tracts misfold and aggregate as antiparallel β-strands termed “polar zippers” (Perutz. 1994. Proc Natl Acad Sci USA, 91:5355). The correlation between the threshold polyQ length for aggregation in experimental systems and the CAG repeat length that leads to human disease supports the argument that self-association or aggregation of expanded polyQ underlies the toxic gain of function. Although in some experimental systems the toxicity of expanded polyQ has been dissociated from the formation of visible inclusions,. the formation of insoluble molecular aggregates appears to be a consistent feature of cellular toxicity (Sisodia. 1998. Cell 95:1; Klement, et al. 1998. Cell 95:41; Saudou, et al. 1998. Cell 95:55; Muchowski, et al. 2002. Proc Natl Acad Sci USA 99:727).

Transgenic mice expressing huntingtin alleles with a diverse set of expanded polyglutamine stretches develop an early clasping phenotype, motor coordination impairment and hyperactivity. These phenotypes are associated with neuropathological appearance of cortical, septal, hippocampal inclusions, reactive gliosis, cell loss, general brain atrophy and cellular inclusions consistent with changes normally seen in Huntington's patients (Schilling, et al. 1999. Hum Mol Genet 8:397; Reddy, et al. 1998. Nat Genet 20:198; DiFiglia, et al. 1997. Science 277:1990; Yamamoto, et al. 2000. Cell 101:57). Similar models have been developed for other Polyglutamine Disorders.

Serpinopathies

The serpinopathies include alpha(1)-antitrypsin (SERPINA1) deficiency and the newly characterized familial encephalopathy with neuroserpin inclusion bodies (FENIB) resulting from mutations in the neuroserpin (SERPINI1) gene.

The serpins, or serine proteinase inhibitors, are a superfamily of proteins found in a large range of species, including viruses, plants and humans. The family includes many diverse members such as α1-antichymotrypsin, C1 inhibitor, antithrombin and plasminogen activator inhibitor-1. In addition to their role in inflammatory, complement, coagulation and fibrinolytic processes, serpins also are involved in chromatin packing and include the proteins MENT and neuroserpin. Inclusion into the serpin superfamily is based on greater than 30% amino-acid homology to α1-antitrypsin and a conserved tertiary structure that is based on three β-sheets (A-C) and an exposed mobile reactive loop that presents a peptide sequence as a pseudosubstrate for the target proteinase.

This conformation is necessary for proteinase function but also renders them liable to undergo conformational transitions that cause disease. Point mutations can destabilize β-sheet A to allow incorporation of the loop of another serpin molecule. Chains of polymers result from sequential reactive loop insertions which are then retained within the cell and cumulatively lead to tissue damage (Stein and Carrell. 1995. Nat Struct Biol 2:96).

This common mechanism has been proposed to explain the variation in disease phenotypes associated with mutations in the members of the serpin superfamily which has led to the grouping of these diverse diseases into a group termed serpinopathies (For a review see, Lomas and Carrell. 2002. Nat Rev Genet 3:759).

Tauopathies

The observation that a broad range of sporadic neuropathological disorders are predominantly characterized by filamentous tau inclusions, similar to that observed in patients with AD and prion disease, has led investigators to propose that tau plays a causative role in these disorders which are collectively referred to as tauopathies. This group of disorders includes Alzheimer's disease, Dementia pugilistica, Down syndrome; Prion diseases, cerebral amyloid angiopathy, Amyotrophic lateral sclerosis/parkinsonism-dementia complex, Argyophilic grain dementia, Corticobasal degeneration, Diffuse neurofibrillary tangles with calcification, Frontotemporal dementia/parkinsonism linked to chromosone-17, Hallervorden-Spatz disease, Multiple system atrophy (MSA), Nieman-Pick disease type C, Pick's disease, Progressive supranuclear palsy, Subacute sclerosing panencephalitis, and Tangle-predominant Alzheimer's disease (Lee and Goedert. 2001. Ann Rev Neurosci 24:1121).

Tau proteins are low molecular weight microtubule-associated proteins that are abundant in the central and peripheral nervous system. The discovery that multiple mutations in the gene encoding tau are associated with frontotemporal dementia and parkinsonism (FTDP-17) provided strong evidence that abnormal forms of tau may contribute to these and potentially other neurodegenerative disease (Reed, et al. 2001. Neurobiol 22:89). Moreover, polymorphisms associated with the tau gene appear to be a risk factor for sporadic corticobasal degeneration, progressive supranuclear palsy and Parkinson's disease (Martin, et al. 2001. J Am Med Assoc 286:2245; Cole, et al. 1999. Semin Neurol. 19:407). The neurofibrilliary tangles (NFTs) (see above), characteristic of AD, also consist of intracellular aggregates of hyperphosphorylated microtubular tau protein. In fact, hyperphosphorylation appears to be a post-translational event favoring the assembly of tau into aggregates.

Although the regions of the brain affected by each of these diseases are different, the evidence suggests that some common features appear to occur in all of them: impaired splicing of tau leading to abnormal hyperphosphorylation of tau, fibrillization of tau and ultimately deposition of tau into aggregates (For a review see, Avila. 2000. FEBS Lett 476:89; Taylor, et al. 2002. Science, 296:1991).

Transgenic mice, expressing the long isoform of Tau bearing mutations found in patients with frontotemporal dementia and parkinsonism develop a tauopathy characterized by the development of congophilic hyperphosphorylated tau inclusions in forebrain neurons. These inclusions appear as early as 18 months of age. As with human cases, tau inclusions are composed of both mutant and endogenous wild-type tau, and are associated with microtubule disruption and flame-shaped transformations of the affected neurons. Behaviorally, aged transgenic mutant Tau mice display cognitive deficits and in particular associative memory impairment (Lewis, et al. 2000. Nat Genet 25:402; Götz, et al. 2001. J Biol Chem 276:529; Tatebayashi, et al. 2002. Proc Natl Acad Sci USA 99:13896; Götz, et al. 2001. Eur J Neurosci. 13:2131; Tanemura, et al. 2002. J Neurosci 22:133; Ishihara, et al. 1999. Neuron 24:751; Spittaels, et al. 1999. Am J Pathol 155:2153; Probst, et al. 2000. Acta Neuropathol 99:469).

Other Related Protein Aggregation Disorders

Amyotrophic Lateral Sclerosis (ALS)

Amyotrophic lateral sclerosis (ALS) is a progressive neurodegenerative disease of upper and lower motor neurons. About 10% of ALS cases are inherited; the remainder are believed to be sporadic cases (Cole, et al. 1999. Semin Neurol, 19:407), and are characterized neuropathologically by degeneration and loss of motor neurons and gliosis. Intracellular inclusions are found in the degenerating neurons and glia (Rowland, et al. 2001. N Eng J Med, 344:1688). Of the inherited cases, approximately 20% are caused by mutations in the gene encoding superoxide dismutase 1 (SOD1). More than 70 different pathogenic SOD1 mutations have been described. Familial ALS is neuropathologically characterized by neuronal Lewy body-like hyaline inclusions and astrocytic hyaline inclusions composed of mutant SOD1. Support for the pathogenic toxicity of mutant SOD1 aggregates is the observation that murine models of mutant SOD1-mediated disease feature prominent intracellular inclusions in motor neurons, and in some cases within the astrocytes surrounding them (Cleveland and Liu. 2000. Nature Med 6: 1320).

Cataracts

Mutations in alpha-crystallin can cause cataract. Alpha-crystallins (α-crystallins) are a major protein component of the mammalian eye lens. They are molecular chaperones that participate in the folding of many proteins and several families are known to exist in mammalian cells including the small heat shock protein (sHSP) family. sHSPs have a molecular mass of about 15-30 kDa. Alpha-crystallin is thought to play a critical role in the maintenance of transparency through its ability to inhibit stress-induced detrimental protein aggregation. α-Crystallin prevents the aggregation of other lens crystallins and proteins that have become unfolded by ‘trapping’ the protein in a high-molecular-mass complex. However, during aging, the chaperone function of α-crystallin becomes compromised, allowing the formation of light-scattering aggregates or inclusion bodies. Within the central part of the lens there is no turnover of damaged protein, and therefore post-translational modifications of α-crystallin accumulate that can further reduce their chaperone function, thus leading to the formation of cataract lenses (For a review see, Horwitz. 2003. Exp Eye Res. 76:145).

Wilson's Disease

Wilson's Disease is a genetic disorder characterized by the accumulation of copper in the body as a result of a defect of copper excretion from hepatocytes. The intracellular localization of the Wilson's disease gene product, ATP7B, was recently identified as the late endosomes. ATP7B is a membrane copper transporter. Various mutations have been documented in patients with Wilson's disease. The clinical manifestations vary greatly among the patients. A common ATP7B mutant, His1069Gln, tagged with green fluorescent protein was expressed in Huh7 and HEK293 cells. This mutant protein did not locate in the late endosomes and was degraded by the proteasomes in the cytoplasm. Furthermore, His1069Gln formed aggresomes composed of the degradates and intermediate filaments at the microtubule-organizing center (Harada, et al. 2001. Gastroenterology 121:1264). Additionally, ubiquitin and the heat shock proteins, hsp70 and hsp90, were shown to co-localize in aggresomes in liver biopsy sections of Wilson's Disease patients (Riley. 2003. Exp Mol Pathol 74:168). Based on the characteristics of the aggresomes in Wilson's Disease patients (localization to the peinuclear area, co-localization with proteasomal subunits, transglutaminase and heat shock proteins) has led to the proposal that Wilson's Disease be included as a member of the Protein Conformation disorders (French. 2001. Gastroenterology 121:1264).

Compounds of the Invention

The invention relates to a method of treating or preventing a Protein Aggregation Disorder, that is not an Amyloid Proteopathy (as defined herein), in a subject (preferably a mammal, more preferably a human) comprising administering to the subject an effective amount of a compound according to the any of the following Formulae, such that a Protein Aggregation Disorder is treated or prevented. In another embodiment, the invention relates to a method of preventing, inhibiting, treating or modulating a Protein Aggregation Disorder, that is not an Amyloid Proteopathy (as defined herein), in a subject (preferably a mammal, more preferably a human) comprising administering to a subject an effective amount of a compound of the invention, such that a Protein Aggregation Disorder is prevented, inhibited, treated or modulated.

One group of example compounds of the invention are alkylsulfonic acids, which may have a structure of the Formula Q—[—Y⁻—X⁺]ₙ, wherein Q is a carrier molecule; Y is SO₃⁻X⁺, OSO₃⁻X⁺, or SSO₃⁻X⁺; and X⁺ is a cationic group such as a positively charged atoms or other moiety. Suitable carrier molecules include carbohydrates, polymers, peptides, peptide derivatives, aliphatic groups, alicyclic groups, heterocyclic groups, aromatic groups or combinations thereof. A carrier molecule can be substituted, e.g., with one or more amino, nitro, halogen, thiol or hydroxy groups. See WO 96/28187, WO 01/85093, and U.S. Pat. No. 5,840,294.

One particular group of compounds of the invention have the following Formula: 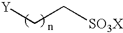
where Y is either an amino group (having the formula —NR^{a}R^{b}) or a sulfonic acid group (having the formula —SO₃⁻X⁺), n is an integer from 1 to 5, and X is hydrogen or a cationic group (e.g., sodium). Some exemplary alkylsulfonic acids include the following

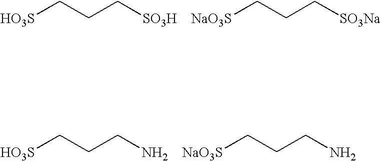

In some cases, the alkylsulfonic acid is a “small molecule,” that is, a compound that that is not itself the product of gene transcription or translation (e.g., protein, RNA, or DNA) and has a low molecular weight, e.g., less than about 2500. In other cases, the compound may be a biological product, such as an antibody or an immunogenic peptide.

Alkylsulfonic acids may be prepared by the methods illustrated in the general reaction schemes as, for example, described in U.S. Pat. Nos. 5,643,562; 5,972,328; 5,728,375; 5,840,294; 4,657,704; and U.S. provisional patent application No. 60/482,058, filed Jun. 23, 2003, U.S. provisional patent application No. 60/512,135, filed Oct. 17, 2003, both entitled Synthetic Process for Preparing Compounds for Treating Amyloidosis, and U.S. application Ser. No. 10/______, filed Jun. 18, 2004, identified by Attorney Docket No. NBI-156, entitled Improved Pharmaceutical Drug Candidates and Method for Preparation Thereof, or by modifications thereof, using readily available starting materials, reagents and conventional synthesis procedures. In these reactions, it is also possible to make use of variants which are in themselves known, but are not mentioned. Functional and structural equivalents of the compounds described herein and which have the same general properties, wherein one or more simple variations of substituents are made which do not adversely affect the essential nature or the utility of the compound may be prepared according to a variety of methods known in the art.

The term “alkylsulfonic acid” as used herein includes substituted or unsubstituted alkylsulfonic acids, and substituted or unsubstituted lower alkylsulfonic acids. Amino-substituted compounds are especially noteworthy and the invention pertains to substituted- or unsubstituted-amino-substituted alkylsulfonic acids, and substituted- or unsubstituted-amino-substituted lower alkylsulfonic acids, and example of which is 3-amino-1-propanesulfonic acid. Also, it should be noted that the term “alkylsulfonic acid” as used herein is to be interpreted as being synonymous with the term “alkanesulfonic acid.”

The invention is directed to a substituted or unsubstituted alkylsulfonic acid, substituted or unsubstituted alkylsulfuric acid, substituted or unsubstituted alkylthiosulfonic acid, substituted or unsubstituted alkylthiosulfiric acid, or an ester or amide thereof, including pharmaceutically acceptable salts thereof. For example, the invention relates to a compound that is a substituted or unsubstituted alkylsulfonic acid, or an ester or amide thereof, including pharmaceutically acceptable salts thereof. In another embodiment, the invention pertains to a compound that is a substituted or unsubstituted lower alkylsulfonic acid, or an ester or amide thereof, including pharmaceutically acceptable salts thereof. Similarly, the invention includes a compound that is a (substituted- or unsubstituted-amino)-substituted alkylsulfonic acid, or an ester or amide thereof, including pharmaceutically acceptable salts thereof. In yet another embodiment, the compound is a (substituted- or unsubstituted-amino)-substituted lower alkylsulfonic acid, or an ester or amide thereof, including pharmaceutically acceptable salts thereof.

As used herein, “alkyl” groups include saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (isopropyl, tert-butyl, sec-butyl, isobutyl, etc.), and alkyl-substituted alkyl groups (e.g., alkyl-substituted cycloalkyl groups and cycloalkyl-substituted alkyl groups). The term “aliphatic group” includes organic moieties characterized by straight or branched-chains, typically having between 1 and 22 carbon atoms. In complex structures, the chains may be branched, bridged, or cross-linked. Aliphatic groups include alkyl groups, alkenyl groups, and alkynyl groups.

Accordingly, the invention relates to substituted or unsubstituted alkylsulfonic acids that are substituted or unsubstituted straight-chain alkylsulfonic acids, substituted or unsubstituted cycloalkylsulfonic acids, and substituted or unsubstituted branched-chain alkylsulfonic acids.

The structures of some of the compounds of this invention include stereogenic carbon atoms. It is to be understood that isomers arising from such asymmetry (e.g., all enantiomers and diastereomers) are included within the scope of this invention unless indicated otherwise. That is, unless otherwise stipulated, any chiral carbon center may be of either (R)- or (S)-stereochemistry. Such isomers can be obtained in substantially pure form by classical separation techniques and by stereochemically-controlled synthesis. In addition, the compounds of the present invention may exist in unsolvated as well as solvated forms with acceptable solvents such as water, THF, ethanol, and the like. In general, the solvated forms are considered equivalent to the unsolvated forms for the purposes of the present invention. The term “solvate” represents an aggregate that comprises one or more molecules of the a compound, with one or more molecules of a pharmaceutical solvent, such as water, ethanol, and the like.

In certain embodiments, a straight-chain or branched-chain alkyl group may have 30 or fewer carbon atoms in its backbone, e.g., C₁-C₃₀ for straight-chain or C₃-C₃₀ for branched-chain. In certain embodiments, a straight-chain or branched-chain alkyl group may have 20 or fewer carbon atoms in its backbone, e.g., C₁-C₂₀ for straight-chain or C₃-C₂₀ for branched-chain, and more, for example, 18 or fewer. Likewise, example cycloalkyl groups have from 4-10 carbon atoms in their ring structure, or 4-7 carbon atoms in the ring structure.

The term “lower alkyl” refers to alkyl groups having from 1 to 6 carbons in the chain, and to cycloalkyl groups having from 3 to 6 carbons in the ring structure. Unless the number of carbons is otherwise specified, “lower” as in “lower alkyl,” means that the moiety has at least one and less than about 8 carbon atoms. In certain embodiments, a straight-chain or branched-chain lower alkyl group has 6 or fewer carbon atoms in its backbone (e.g., C₁-C₆ for straight-chain, C₃-C₆ for branched-chain), for example, methyl, ethyl, propyl, isopropyl, butyl, isobutyl, sec-butyl, and tert-butyl. Likewise, cycloalkyl groups may have from 3-8 carbon atoms in their ring structure, for example, 5 or 6 carbons in the ring structure. The term “C1-C6” as in “C1-C6 alkyl” means alkyl groups containing 1 to 6 carbon atoms.

Moreover, unless otherwise specified the term alkyl includes both “unsubstituted alkyls” and “substituted alkyls,” the latter of which refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclic, alkylaryl, or aromatic (including heteroaromatic) groups.

An “arylalkyl” group is an alkyl group substituted with an aryl group (e.g., phenylmethyl (i.e., benzyl)). An “alkylaryl” moiety is an aryl group substituted with an alkyl group (e.g., p-methylphenyl (i.e., p-tolyl)). The term “n-alkyl” means a straight-chain (i.e., unbranched) unsubstituted alkyl group. An “alkylene” group is a divalent analog of the corresponding alkyl group. The terms “alkenyl” and “alkynyl” refer to unsaturated aliphatic groups analogous to alkyls, but which contain at least one double or triple carbon-carbon bond respectively. Suitable alkenyl and alkynyl groups include groups having 2 to about 12 carbon atoms, preferably from 2 to about 6 carbon atoms.

The term “aromatic group” or “aryl group” includes unsaturated and aromatic cyclic hydrocarbons as well as unsaturated and aromatic heterocycles containing one or more rings. Aryl groups may also be fused or bridged with alicyclic or heterocyclic rings that are not aromatic so as to form a polycycle (e.g., tetralin). An “arylene” group is a divalent analog of an aryl group. Aryl groups can also be fused or bridged with alicyclic or heterocyclic rings which are not aromatic so as to form a polycycle (e.g., tetralin).

The term “heterocyclic group” includes closed ring structures analogous to carbocyclic groups in which one or more of the carbon atoms in the ring is an element other than carbon, for example, nitrogen, sulfur, or oxygen. Heterocyclic groups may be saturated or unsaturated. Additionally, heterocyclic groups (such as pyrrolyl, pyridyl, isoquinolyl, quinolyl, purinyl, and furyl) may have aromatic character, in which case they may be referred to as “heteroaryl” or “heteroaromatic” groups.

Unless otherwise stipulated, aryl and heterocyclic (including heteroaryl) groups may also be substituted at one or more constituent atoms. Examples of heteroaromatic and heteroalicyclic groups may have 1 to 3 separate or fused rings with 3 to about 8 members per ring and one or more N, O, or S heteroatoms. In general, the term “heteroatom” includes atoms of any element other than carbon or hydrogen, preferred examples of which include nitrogen, oxygen, sulfur, and phosphorus. Heterocyclic groups may be saturated or unsaturated or aromatic.

Examples of heterocycles include, but are not limited to, acridinyl; azocinyl; benzimidazolyl; benzofuranyl; benzothiofrranyl; benzothiophenyl; benzoxazolyl; benzthiazolyl; benztriazolyl; benztetrazolyl; benzisoxazolyl; benzisothiazolyl; benzimidazolinyl; carbazolyl; 4aH-carbazolyl; carbolinyl; chromanyl; chromenyl; cinnolinyl; decahydroquinolinyl; 2H,6H-1,5,2-dithiazinyl; dihydrofuro[2,3-b]tetrahydrofuran; furanyl; furazanyl; imidazolidinyl; imidazolinyl; imidazolyl; 1H-indazolyl; indolenyl; indolinyl; indolizinyl; indolyl; 3H-indolyl; isobenzofuranyl; isochromanyl; isoindazolyl; isoindolinyl; isoindolyl; isoquinolinyl; isothiazolyl; isoxazolyl; methylenedioxyphenyl; morpholinyl; naphthyridinyl; octahydroisoquinolinyl; oxadiazolyl; 1,2,3-oxadiazolyl; 1,2,4-oxadiazolyl; 1,2,5-oxadiazolyl; 1,3,4-oxadiazolyl; oxazolidinyl; oxazolyl; oxazolidinyl; pyrimidinyl; phenanthridinyl; phenanthrolinyl; phenazinyl; phenothiazinyl; phenoxathiinyl; phenoxazinyl; phthalazinyl; piperazinyl; piperidinyl; piperidonyl; 4-piperidonyl; piperonyl; pteridinyl; purinyl, pyranyl; pyrazinyl; pyrazolidinyl; pyrazolinyl; pyrazolyl; pyridazinyl; pyridooxazole; pyridoimidazole; pyridothiazole; pyridinyl; pyridyl; pyrimidinyl; pyrrolidinyl; pyrrolinyl; 2H-pyrrolyl; pyrrolyl; quinazolinyl; quinolinyl; 4H-quinolizinyl; quinoxalinyl; quinuclidinyl; tetrahydrofuranyl; tetrahydroisoquinolinyl; tetrahydroquinolinyl; tetrazolyl; 6H-1,2,5-thiadiazinyl; 1,2,3-thiadiazolyl; 1,2,4-thiadiazolyl; 1,2,5-thiadiazolyl; 1,3,4-thiadiazolyl; thianthrenyl; thiazolyl; thienyl; thienothiazolyl; thienooxazolyl; thienoimidazolyl; thiophenyl; triazinyl; 1,2,3-triazolyl; 1,2,4-triazolyl; 1,2,5-triazolyl; 1,3,4-triazolyl; and xanthenyl. Preferred heterocycles include, but are not limited to, pyridinyl; furanyl; thienyl; pyrrolyl; pyrazolyl; pyrrolidinyl; imidazolyl; indolyl; benzimidazolyl; 1H-indazolyl; oxazolidinyl; benzotriazolyl; benzisoxazolyl; oxindolyl; benzoxazolinyl; and isatinoyl groups. Also included are fused ring and spiro compounds containing, for example, the above heterocycles.

A common hydrocarbon aryl group is a phenyl group having one ring. Two-ring hydrocarbon aryl groups include naphthyl, indenyl, benzocyclooctenyl, benzocycloheptenyl, pentalenyl, and azulenyl groups, as well as the partially hydrogenated analogs thereof such as indanyl and tetrahydronaphthyl. Exemplary three-ring hydrocarbon aryl groups include acephthylenyl, fluorenyl, phenalenyl, phenanthrenyl, and anthracenyl groups.

Aryl groups also include heteromonocyclic aryl groups, i.e., single-ring heteroaryl groups, such as thienyl, furyl, pyranyl, pyrrolyl, imidazolyl, pyrazolyl, pyridinyl, pyrazinyl, pyrimidinyl, and pyridazinyl groups; and oxidized analogs thereof such as pyridonyl, oxazolonyl, pyrazolonyl, isoxazolonyl, and thiazolonyl groups. The corresponding hydrogenated (i.e., non-aromatic) heteromonocylic groups include pyrrolidinyl, pyrrolinyl, imidazolidinyl, imidazolinyl, pyrazolidinyl, pyrazolinyl, piperidyl and piperidino, piperazinyl, and morpholino and morpholinyl groups.

Aryl groups also include fused two-ring heteroaryls such as indolyl, isoindolyl, indolizinyl, indazolyl, quinolinyl, isoquinolinyl, phthalazinyl, quinoxalinyl, quinazolinyl, cinnolinyl, chromenyl, isochromenyl, benzothienyl, benzimidazolyl, benzothiazolyl, purinyl, quinolizinyl, isoquinolonyl, quinolonyl, naphthyridinyl, and pteridinyl groups, as well as the partially hydrogenated analogs such as chromanyl, isochromanyl, indolinyl, isoindolinyl, and tetrahydroindolyl groups. Aryl groups also include fused three-ring groups such as phenoxathiinyl, carbazolyl, phenanthridinyl, acridinyl, perimidinyl, phenanthrolinyl, phenazinyl, phenothiazinyl, phenoxazinyl, and dibenzofuranyl groups.

Some typical aryl groups include substituted or unsubstituted 5- and 6-membered single-ring groups. In another aspect, each Ar group may be selected from the group consisting of substituted or unsubstituted phenyl, pyrrolyl, fliryl, thienyl, thiazolyl, isothiaozolyl, imidazolyl, triazolyl, tetrazolyl, pyrazolyl, oxazolyl, isooxazolyl, pyridinyl, pyrazinyl, pyridazinyl, and pyrimidinyl groups. Further examples include substituted or unsubstituted phenyl, 1-naphthyl, 2-naphthyl, biphenyl, 1-pyrrolyl, 2-pyrrolyl, 3-pyrrolyl; 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl groups.

The term “amine” or “amino,” as used herein, refers to an unsubstituted or substituted moiety of the formula —NR^{a}R^{b}, in which R^{a} and R^{b} are each independently hydrogen, alkyl, aryl, or heterocyclyl, or R^{a} and R^{b}, taken together with the nitrogen atom to which they are attached,form a cyclic moiety having from 3 to 8 atoms in the ring. Thus, the term amino includes cyclic amino moieties such as piperidinyl or pyrrolidinyl groups, unless otherwise stated. Thus, the term “alkylamino” as used herein means an alkyl group having an amino group attached thereto. Suitable alkylamino groups include groups having 1 to about 12 carbon atoms, for example, 1 to about 6 carbon atoms. The term amino includes compounds or moieties in which a nitrogen atom is covalently bonded to at least one carbon or heteroatom. The term “dialkylamino” includes groups wherein the nitrogen atom is bound to at least two alkyl groups. The term “arylamino and diarylamino” include groups wherein the nitrogen is bound to at least one or two aryl groups, respectively. The term “alkylarylamino” refers to an amino group which is bound to at least one alkyl group and at least one aryl group. The term “alkaminoalkyl” refers to an alkyl, alkenyl, or alkynyl group substituted with an alkylamino group. The term “amide” or “aminocarbonyl” includes compounds or moieties which contain a nitrogen atom which is bound to the carbon of a carbonyl or a thiocarbonyl group.

The term “alkylthio” refers to an alkyl group, having a sulfhydryl group attached thereto. Suitable alkylthio groups include groups having 1 to about 12 carbon atoms, preferably from 1 to about 6 carbon atoms.

The term “alkylcarboxyl” as used herein means an alkyl group having a carboxyl group attached thereto.

The term “alkoxy” as used herein means an alkyl group having an oxygen atom attached thereto. Representative alkoxy groups include groups having 1 to about 12 carbon atoms, preferably 1 to about 6 carbon atoms, e.g., methoxy, ethoxy, propoxy, tert-butoxy and the like. Examples of alkoxy groups include methoxy, ethoxy, isopropyloxy, propoxy, butoxy, and pentoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfmyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc., as well as perhalogenated alkyloxy groups.

The term “acylamino” includes moieties wherein an amino moiety is bonded to an acyl group. For example, the acylamino group includes alkylcarbonylamino, arylcarbonylamino, carbamoyl and ureido groups.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone.

The term “carbonyl” or “carboxy” includes compounds and moieties which contain a carbon connected with a double bond to an oxygen atom. Examples of moieties which contain a carbonyl include aldehydes, ketones, carboxylic acids, amides, esters, anhydrides, etc.

The term “ether” or “ethereal” includes compounds or moieties which contain oxygen bonded to two carbon atoms. For example, an ether or ethereal group includes “alkoxyalkyl” which refers to an alkyl, alkenyl, or alkynyl group substituted with an alkoxy group.

A “sulfonic acid” or “sulfonate” group is a —SO₃H or —SO₃⁻X⁺ group bonded to a carbon atom, where X⁺ is a cationic counter ion group. Similarly, a “sulfonic acid” compound has a —SO₃H or —SO₃⁻X⁺ group bonded to a carbon atom, where X⁺ is a cationic group. A “sulfate” as used herein is a —OSO₃H or —OSO₃⁻X⁺ group bonded to a carbon atom, and a “sulfuric acid” compound has a —SO₃H or —OSO₃⁻X⁺ group bonded to a carbon atom, where X⁺ is a cationic group. According to the invention, a suitable cationic group may be a hydrogen atom. In certain cases, the cationic group may actually be another group on the therapeutic compound that is positively charged at physiological pH, for example and amino group.

A “counter ion” is required to maintain electroneutrality, and is pharmaceutically acceptable in the compositions of the invention. Compounds containing a cationic group covalently bonded to an anionic group may be referred to as an “internal salt.” Examples of anionic counter ions include halide, triflate, sulfate, nitrate, hydroxide, carbonate, bicarbonate, acetate, phosphate, oxalate, cyanide, alkylcarboxylate, N-hydroxysuccinimide, N-hydroxybenzotriazole, alkoxide, thioalkoxide, alkane sulfonyloxy, halogenated alkane sulfonyloxy, arylsulfonyloxy, bisulfate, oxalate, valerate, oleate, palmitate, stearate, laurate, borate, benzoate, lactate citrate, maleate, fumarate, succinate, tartrate, naphthylate mesylate, glucoheptonate, or lactobionate. Compounds containing a cationic group covalently bonded to an anionic group may be referred to as an “internal salt.”

The term “nitro” means —NO₂; the term “halogen” or “halogeno” or “halo” designates —F, —Cl, —Br or —I; the term “thiol,” “thio,” or “mercapto” means SH; and the term “hydroxyl” or “hydroxy” means —OH.

The term “acyl” refers to a carbonyl group that is attached through its carbon atom to a hydrogen (i e., a formyl), an aliphatic group (e.g., acetyl), an aromatic group (e.g., benzoyl), and the like. The term “substituted acyl” includes acyl groups where one or more of the hydrogen atoms on one or more carbon atoms are replaced by, for example, an alkyl group, alkynyl group, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic. moiety.

Unless otherwise specified, the chemical moieties of the compounds of the invention, including those groups discussed above, may be “substituted or unsubstituted.” In some embodiments, the term “substituted” means that the moiety has substituents placed on the moiety other than hydrogen (i e., in most cases, replacing a hydrogen), which allow the molecule to perform its intended function. Examples of substituents include moieties selected from straight or branched alkyl (e.g., C₁-C₅), cycloalkyl (e.g., C₃-C₈), amino groups (including —NH₂), —SO₃H, —OSO₃H, —CN, —NO₂, halogen (e.g., —F, —Cl, —Br, or —I), —CH₂OCH₃, —OCH₃, —SH, —SCH₃, —OH, and —CO₂H. Examples of substituents include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, and heteroaryl groups, as well as (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), and (CR′R″)₀₋₃OR′ groups, wherein R′ and R″ are each independently hydrogen, a C₁-C₅ alkyl, C₂-C₅ alkenyl, C₂-C₅ alkynyl, or aryl group; or the side chain of any naturally occurring amino acid.

In another embodiment, a substituent may be selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₁₀NR′R″ (e.g., —NH₂), (CR′R″)₀₋₁₀CN (e.g., —CN), NO₂, halogen (e.g., F, Cl, Br, or I), (CR′R″)₀₋₁₀C(halogen)₃ (e.g., —CF₃), (CR′R″)₀₋₁₀CH(halogen)₂(CR′R″)₀₋₁₀CH₂(halogen), (CR′R″)₀₋₁₀CONR′R″, (CR′R″)₀₋₁(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′ (e.g., —SO₃H), (CR′R″)₀₋₁₀O(CR′R″)₀₋₁₀H (e.g., —CH₂OCH₃ and —OCH₃), (CR′R″)₀₋₁₀S(CR′R″)₀₋₃H (e.g., —SH and —SCH₃), (CR′R″)₀₋₁₀OH (e.g., —OH), (CR′R″)₀₋₁₀COR′, (CR′R″)₀₋₁₀ (substituted or unsubstituted phenyl), (CR′R″)₀₋₁₀(C₃-C₈ cycloalkyl), (CR′R″)₀₋₁₀CO₂R′ (e.g., —CO₂H), or (CR′R″)₀₋₁₀OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group.

It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, 0elimination, etc. As used herein, the term “substituted” is meant to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is meant to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more and the same or different for appropriate organic compounds.

In some embodiments, a “substituent” may be, selected from the group consisting of, for example, halogeno, trifluoromethyl, nitro, cyano, C₁-C₆ alkyl, C₂-C₆ alkenyl, C₂-C₆ alkynyl, C₁-C₆ alkylcarbonyloxy, arylcarbonyloxy, C₁-C₆ alkoxycarbonyloxy, aryloxycarbonyloxy, C₁-C₆ alkylcarbonyl, C₁-C₆ alkoxycarbonyl, C₁-C₆ alkoxy, C₁-C₆ alkylthio, arylthio, heterocyclyl, aralkyl, and aryl (including heteroaryl) groups.

Further examples of compounds that may be used as a compound according to the present invention include those described in the U.S. provisional patent application No. 60/480,906, filed Jun. 23, 2003, U.S. provisional patent application No. 60/512,047, filed Oct. 17, 2003, U.S. application Ser. No. 10/______, filed Jun. 18, 2004, identified by Attorney Docket No. NBI-162A, U.S. application Ser. No. 10/______, filed Jun. 18, 2004, identified by Attorney Docket No. NBI-162B, all entitled Methods and Compositions for Treating Amyloid-Related Diseases. Exemplary compounds are also described in U.S. Provisional Patent Application Ser. No. 60/512,018, filed on Oct. 17, 2003, U.S. Provisional Patent Application Ser. No. 60/480,928, filed Jun. 23, 2003, and U.S. patent application Ser. No. 10/______, filed Jun. 18, 2004, identified by Attorney Docket No. NBI-163, all entitled Methods and Compositions for Treating Amyloid- and Epileptogenesis-Associated Diseases.

In an embodiment, the invention pertains, at least in part to a composition having a compound that is a compound of Formula I-A: 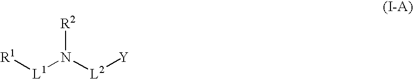
wherein:

R¹ is a substituted or unsubstituted cycloalkyl, aryl, arylcycloalkyl, bicyclic or tricyclic ring, a bicyclic or tricyclic fused ring group, or a substituted or unsubstituted C₂-C₁₀ alkyl group;

R² is selected from a group consisting hydrogen, alkyl, mercaptoalkyl, alkenyl, alkynyl, cycloalkyl, aryl, arylalkyl, thiazolyl, triazolyl, imidazolyl, benzothiazolyl, and benzoimidazolyl;

Y is SO₃⁻X⁺, OSO₃⁻X⁺, or SSO₃⁻X⁺;

X⁺ is hydrogen, a cationic group, or an ester-forming group (i.e., as in a prodrug, which are described elsewhere herein); and

each of L¹ and L² is independently a substituted or unsubstituted C₁-C₅ alkyl group or absent, or a pharmaceutically acceptable salt thereof, provided that when R¹ is alkyl, L¹ is absent.

In another embodiment, the invention pertains, at least in part a composition having a compound that is a compound of Formula II-A: 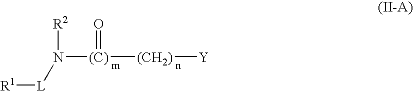
wherein:

R¹ is a substituted or unsubstituted cyclic, bicyclic, tricyclic, or benzoheterocyclic group or a substituted or unsubstituted C₂-C₁₀ alkyl group;

R² is hydrogen, alkyl, mercaptoalkyl, alkenyl, alkynyl, cycloalkyl, aryl, arylalkyl, thiazolyl, triazolyl, imidazolyl, benzothiazolyl, benzoimidazolyl, or linked to R¹ to form a heterocycle;

Y is SO₃⁻X⁺, OSO₃⁻X⁺, or SSO₃⁻X⁺;

X⁺ is hydrogen, a cationic group, or an ester forming moiety;

m is 0 or 1;

n is 1, 2, 3, or 4;

L is substituted or unsubstituted C₁-C₃ alkyl group or absent, or a pharmaceutically acceptable salt thereof, provided that when R¹ is alkyl, L is absent.

In yet another embodiment, the invention pertains, at least in part to a composition having a compound that is a compound of Formula III-A: 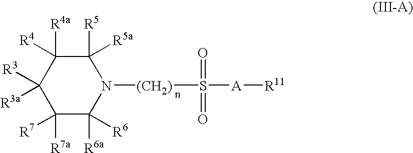
wherein:

A is nitrogen or oxygen;

R¹¹ is hydrogen, salt-forming cation, ester forming group, —(CH₂)ₓ—Q, or when A is nitrogen, A and R¹¹ taken together may be the residue of a natural or unnatural amino acid residue or a salt or ester thereof;

Q is hydrogen, thiazolyl, triazolyl, imidazolyl, benzothiazolyl, or benzoimidazolyl;

x is 0, 1, 2, 3, or 4;

n is 0, 1 , 2, 3, 4, 5, 6, 7, 8, 9, or 10;

R³, R^{3a}, R⁴, R^{4a}, R⁵; R^{5a}, R⁶, R^{6a}, R⁷ and R^{7a} are each independently hydrogen, alkyl, mercaptoalkyl, alkenyl, alkynyl, cycloalkyl, aryl, alkylcarbonyl, arylcarbonyl, alkoxycarbonyl, cyano, halogen, amino, tetrazolyl, or two R groups on adjacent ring atoms taken together with the ring atoms form a double bond, provided that one of R³, R^{3a}, R⁴, R^{4a}, R⁵, R^{5a}, R⁶, R^{6a}, R⁷ and R^{7a} is a moiety of Formula IIIa-A: 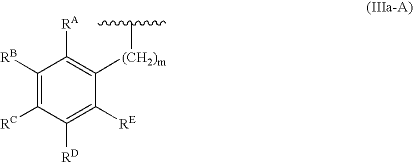
wherein:

m is 0, 1, 2, 3, or 4;

R^{A}, R^{B}, R^{C}, R^{D}, and R^{E} are independently selected from a group of hydrogen, halogen, hydroxyl, alkyl, alkoxyl, halogenated alkyl, mercaptoalkyl, alkenyl, alkynyl, cycloalkyl, aryl, cyano, thiazolyl, triazolyl, imidazolyl, tetrazolyl, benzothiazolyl, and benzoimidazoly; and pharmaceutically acceptable salts and esters thereof, provided that said compound is not 3-(4-phenyl-1, 2, 3, 6-tetrahydro-1-pyridyl)-1-propanesulfonic acid.

In yet another embodiment, the invention pertains at least in part to a composition having a compound that is a compound of Formula IV-A: 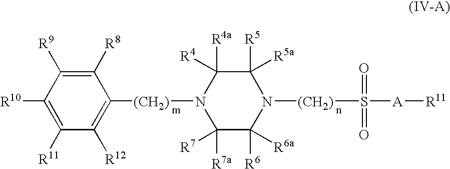
wherein:

A is nitrogen or oxygen;

R¹¹ is hydrogen, salt-forming cation, ester forming group, —(CH₂)ₓ—Q, or when A is nitrogen, A and R¹¹ taken together may be the residue of a natural or unnatural amino acid residue or a salt or ester thereof;

Q is hydrogen, thiazolyl, triazolyl, imidazolyl, benzothiazolyl, or benzoimidazolyl;

x is 0, 1, 2, 3, or 4;

n is 0, 1, 2, 3, 4, 5, 6, 7, 8, 9, or 10;

R⁴, R^{4a}, R⁵, R^{5a}, R⁶, R^{6a}, R⁷, and R^{7a} are each independently hydrogen, alkyl, mercaptoalkyl, alkenyl, alkynyl, cycloalkyl, aryl, alkylcarbonyl, arylcarbonyl, alkoxycarbonyl, cyano, halogen, amino, tetrazolyl, R⁴ and R⁵ are taken together, with the ring atoms they are attached to, form a double bond, or R⁶ and R⁷ are taken together, with the ring atoms they are attached to, form a double bond;

m is 0, 1, 2, 3, or 4;

R⁸, R⁹, R¹⁰, R¹¹, and R¹² are independently selected from a group of hydrogen, halogen, hydroxyl, alkyl, alkoxyl, halogenated alkyl, mercaptoalkyl, alkenyl, alkynyl, cycloalkyl, aryl, cyano, thiazolyl, triazolyl, imidazolyl, tetrazolyl, benzothiazolyl, and benzoimidazolyl, and pharmaceutically acceptable salts and esters thereof.

In another embodiment, the invention includes a composition having a compound that is a compound of Formula V-A: 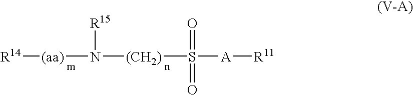
wherein:

A is nitrogen or oxygen;

R¹¹ is hydrogen, salt-forming cation, ester forming group, —(CH₂)ₓ—Q, or when A is nitrogen, A and R¹¹ taken together may be the residue of a natural or unnatural anino acid residue or a salt or ester thereof;

Q is hydrogen, thiazolyl, triazolyl, imidazolyl, benzothiazolyl, or benzoimidazolyl;

x is 0, 1, 2, 3, or 4;

n is 0, 1, 2, 3, 4, 5, 6, 7, 8, 9, or 10;

aa is a natural or unnatural amino acid residue;

m is 0, 1, 2, or 3;

R¹⁴ is hydrogen or protecting group;

R¹⁵ is hydrogen, alkyl or aryl, and pharmaceutically acceptable salts and prodrugs thereof.

In another embodiment, the invention includes a composition having a compound that is a compound of Formula VI-A: 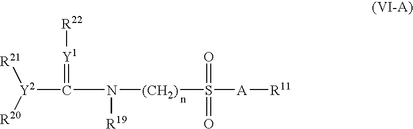
wherein:

n is 1, 2, 3, 4, 5, 6, 7, 8, 9, or 10;

A is oxygen or nitrogen;

R¹¹ is hydrogen, salt-forming cation, ester forming group, —(CH₂)ₓ—Q, or when A is nitrogen, A and R¹¹ taken together may be the residue of a natural or unnatural amino acid residue or a salt or ester thereof;

Q is hydrogen, thiazolyl, triazolyl, imidazolyl, benzothiazolyl, or benzoimidazolyl;

x is 0, 1, 2, 3, or 4;

R¹⁹ is hydrogen, alkyl or aryl;

Y¹ is oxygen, sulfur, or nitrogen;

Y² is carbon, nitrogen, or oxygen;

R²⁰ is hydrogen, alkyl, amino, mercaptoalkyl, alkenyl, alkynyl, cycloalkyl, aryl, arylalkyl, thiazolyl, triazolyl, tetrazolyl, imidazolyl, benzothiazolyl, or benzoimidazolyl;

R²¹ is hydrogen, alkyl, mercaptoalkyl, alkenyl, alkynyl, cycloalkyl, aryl, arylalkyl, thiazolyl, triazolyl, tetrazolyl, imidazolyl, benzothiazolyl, benzoimidazolyl, or absent if Y² is oxygen;

R²² is hydrogen, alkyl, mercaptoalkyl, alkenyl, alkynyl, cycloalkyl, aryl, arylalkyl, thiazolyl, triazolyl, tetrazolyl, imidazolyl, benzothiazolyl, benzoimidazolyl; or R²² is hydrogen, hydroxyl, alkoxy or aryloxy if Y¹ is nitrogen; or R²² is absent if Y¹ is oxygen or sulfur; or R²² and R²¹ may be linked to form a cyclic moiety if Y¹ is nitrogen;

or pharmaceutically acceptable salts thereof.

In another embodiment, the invention includes a composition having a compound that is a compound of Formula VII-A: 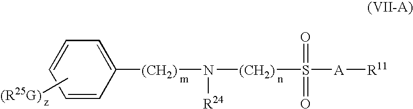
wherein:

n is 2, 3,or 4;

A is oxygen or nitrogen;

R¹ is hydrogen, salt-forming cation, ester forming group, —(CH₂)x—Q, or when A is nitrogen, A and R¹¹ taken together may be the residue of a natural or unnatural amino acid residue or a salt or ester thereof;

Q is hydrogen, thiazolyl, triazolyl, imidazolyl, benzothiazolyl, or benzoimidazolyl;

x is 0, 1, 2, 3, or 4;

G is a direct bond or oxygen, nitrogen, or sulfur;

z is 0, 1, 2, 3, 4, or 5;

m is 0 or 1;

R²⁴ is selected from a group consisting hydrogen, alkyl, mercaptoalkyl, alkenyl, alkynyl, aroyl, alkylcarbonyl, aminoalkylcarbonyl, cycloalkyl, aryl, arylalkyl, thiazolyl, triazolyl, imidazolyl, benzothiazolyl, and benzoimidazolyl;

each R²⁵ is independently selected from hydrogen, halogen, cyano, hydroxyl, alkoxy, thiol, amino, nitro, alkyl, aryl, carbocyclic, or heterocyclic, and pharmaceutically acceptable salts thereof.

Additional compounds include, for example, compounds of Formula I-B: 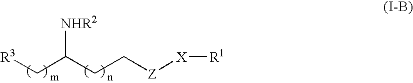
wherein:

X is oxygen or nitrogen;

Z is C═O, S(O)₂, or P(O)OR⁷;

m and n are each independently 0, 1, 2, 3, 4, 5, 6, 7, 8, 9 or 10;

R¹ and R⁷ are each independently hydrogen, metal ion, alkyl, mercaptoalkyl, alkenyl, alkynyl, cycloalkyl, aryl, a moiety together with X to form natural or unnatural amino acid residue, or —(CH₂)ₚ—Y;

Y is hydrogen or a heterocyclic moiety selected from the group consisting of thiazolyl, triazolyl, tetrazolyl, imidazolyl, benzothiazolyl, and benzoimidazolyl;

p is 0, 1, 2, 3, or 4;

R² is hydrogen, alkyl, mercaptoalkyl, alkenyl, alkynyl, cycloalkyl, aryl, alkylcarbonyl, arylcarbonyl, or alkoxycarbonyl;

R³ is hydrogen, amino, cyano, alkyl, mercaptoalkyl, alkenyl, alkynyl, cycloalkyl, heterocyclic, sunbstituted or unsubstituted aryl, heteroaryl, thiazolyl, triazolyl, tetrazolyl, imidazolyl, benzothiazolyl, or benzoimidazolyl, and pharmaceutically acceptable salts, esters, and prodrugs thereof.

In a further embodiment, m is 0, 1, or 2. In another further embodiment, n is 0, 1, or 2. In another further embodiment, R³ is aryl, e.g., heteroaryl or phenyl. In yet another embodiment; Z is S(O)₂.

In another embodiment, the compound of the invention is of the Formula II-B: 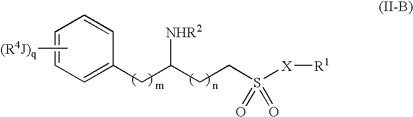
wherein:

each R⁴ is independently selected from the group consisting of hydrogen, halogen, hydroxyl, thiol, amino, cyano, nitro, alkyl, aryl, carbocyclic or heterocyclic;

J is absent, oxygen, nitrogen, sulfur, or a divalent link-moiety comsisting of, without limiting to, lower alkylene, alkylenyloxy, alkylenylamino, alkylenylthio, alkylenyloxyalkyl, alkylenylamonialkyl, alkylenylthioalkyl, alkenyl, alkenyloxy, alkenylamino, or alkenylthio; and

q is 1, 2, 3, 4, or 5, and pharmaceutically acceptable salts, esters and prodrugs thereof, wherein the remaining constituents are as defined above.

In a yet further embodiment, the compound of the invention is of Formula III-B: 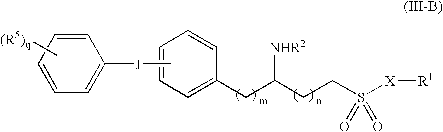
wherein:

X is oxygen or nitrogen;

m and n are each independently 0, 1, 2, 3, 4, 5, 6, 7, 8, 9 or 10;

q is 1, 2, 3, 4, or 5;

R¹ is hydrogen, metal ion, alkyl, mercaptoalkyl, alkenyl, alkynyl, cycloalkyl, aryl, or a moiety together with X to form a natural or unnatural amino acid residue, or —(CH₂)ₚ—Y;

Y is hydrogen or a heterocyclic moiety selected from the group consisting of thiazolyl, triazolyl, tetrazolyl, imidazolyl, benzothiazolyl, and benzoimidazolyl;

p is 0, 1, 2, 3, or 4;

R² is hydrogen, alkyl, mercaptoalkyl, alkenyl, alkynyl, cycloalkyl, aryl, alkylcarbonyl, arylcarbonyl, or alkoxycarbonyl;

R⁵ is selected from the group consisting of hydrogen, halogen, amino, nitro, hydroxy, carbonyl, thiol, carboxy, alkyl, alkoxy, alkoxycarbonyl, acyl, alkylamino, acylamino;

q is an integer selected from 1 to 5;

J is absent, oxygen, nitrogen, sulfur, or a divalent link-moiety comsisting of, without limiting to, lower alkylene, alkylenyloxy, alkylenylamino, alkylenylthio, alkylenyloxyalkyl, alkylenylamonialkyl, alkylenylthioalkyl, alkenyl, alkenyloxy, alkenylamino, or alkenylthio; and

pharmaceutically acceptable salts, esters, and prodrugs thereof.

In yet another embodiment, the compound of the invention is:

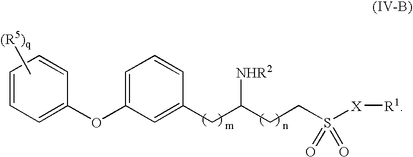

In a further embodiment, m is 0.

In another embodiment, the invention pertains to compounds of Formula V-B: 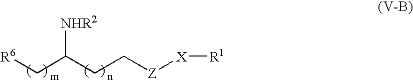
wherein:

R⁶ is a substituted or unsubstituted heterocyclic moiety, and the remaining constituents are as defined above.

In a further embodiment, m is 0 or 1. In another embodiment, n is 0 or 1. In another further embodiment, R⁶ is thiazolyl, oxazoylyl, pyrazolyl, indolyl, pyridinyl, thiazinyl, thiophenyl, benzothiophenyl, dihydroimidazolyl, dihydrothiazolyl, oxazolidinyl, thiazolidinyl, tetrahydropyrimidinyl, or oxazinyl. In yet another embodiment, Z is S(O)₂.

The invention also realtes to the compounds of Formula I-C (see WO 00/64,420): 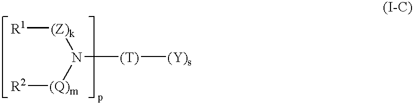
wherein R¹ and R² are each independently a hydrogen atom or a substituted or unsubstituted aliphatic or aryl group; Z and Q are each independently a carbonyl (C═O), thiocarbonyl (C═S), sulfonyl (SO₂), or sulfoxide (S═O) group; k and m are 0 or 1, provided when k is 1, R¹ is not a hydrogen atom, and when m is 1, R² is not a hydrogen atom. In an embodiment, at least one of k or m must equal 1. The variables p and s are each independently positive integers selected such that the biodistribution of the therapeutic compound for an intended target site is not prevented while maintaining activity of the therapeutic compound. T is a linking group (such as an a;kylene group) and Y is a group of the formula is SO₃⁻X⁺OSO₃⁻X⁺, or SSO₃⁻X⁺; wherein X⁺ is a cationic group; and pharmaceutically acceptable salts and prodrugs thereof.

In another embodiment of Formula I-C, R¹ is an alkyl, alkenyl, or a single-ring aromatic group, where said alkyl group may be substituted with a hydroxyl group; R² is a alkyl, alkenyl, hydroxyalkyl, a single-ring aromatic group, or a hydrogen atom, or R¹ and R², taken together with the nitrogen to which they are attached, form a heterocyclic group which is a fused ring structure; k and m are zero, and p and s are one; T is an alkylene group; Y is SO₃X, and X is a cationic group; and pharmaceutically acceptable salts and prodrugs thereof.

In yet another embodiment of Formula I-C, R¹ is an alkyl, an alkenyl, or an aromatic group; R² is a hydrogen atom, an alkyl group, or an aromatic group, or R¹ and R², taken together, form a heterocyclic group which is a fused ring structure; Z and Q are each independently a carbonyl (C═O), thiocarbonyl (C═S), sulfonyl (SO₂), or sulfoxide (S═O) .group; k is 1 and m is 0 or 1, provided when k is 1, R¹ is not a hydrogen atom and when m is 1, R² is not a hydrogen atom; p and s are each 1; T is an alkylene group; and Y is SO₃X, and X is a cationic group; and pharmaceutically acceptable salts thereof.

In another aspect, compounds of Formula I-C may be have R¹ and R² as alkyl, alkenyl, or single-ring aromatic groups, or R¹ and R², taken together with the nitrogen to which they are attached, form a heterocyclic group which is a fused ring structure; k and m as zero, and p and s as one; T as an alkylene group; Y as SO₃X, and X as a cationic group; and pharmaceutically acceptable salts and prodrugs thereof.

In still yet another embodiment of Formula I-C, R¹ is an alkyl, alkenyl, or single-ring aromatic group, where said alkyl group may be substituted with a hydroxyl group; R² is a alkyl, alkenyl, single-ring aromatic group, or a hydrogen atom, where said alkyl group may be substituted with a hydroxyl group, or R¹ and R², taken together with the nitrogen to which they are attached, form a heterocyclic group which is a fused ring structure; k and m are zero, and p and s are one; T is an alkylene group; Y is SO₃X, and X is a cationic group; and pharmaceutically acceptable salts and prodrugs thereof.

In still yet another embodiment, the invention pertains to compounds of Formula I-D: 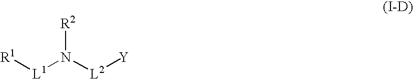
wherein:

R¹ is a substituted or unsubstituted cycloalkyl, heterocyclic, aryl, arylcycloalkyl, bicyclic or tricyclic ring, a bicyclic or tricyclic fused ring group, or a substituted or unsubstituted C₂-C₁₀ alkyl group;

R² is selected from a group consisting of hydrogen, alkyl, mercaptoalkyl, alkenyl, alkynyl, cycloalkyl, aryl, arylalkyl, thiazolyl, triazolyl, imidazolyl, benzothiazolyl, and benzoimidazolyl;

Y is SO₃⁻X⁺, OSO₃⁻X⁺or SSO₃⁻X⁺;

X⁺ is hydrogen, a cationic group, or ester-forming group; and

each of L¹ and L² is independently a substituted or unsubstituted C₁-C₅ alkyl group or absent, or a pharmaceutically acceptable salt thereof, provided that when R₁ is alkyl, L¹ is absent.

In a further embodiment, the invention pertains to compounds of Formula II-D: 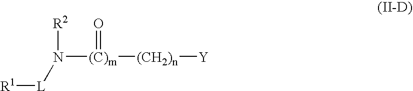
wherein:

R¹ is a substituted or unsubstituted cyclic, bicyclic, tricyclic, or benzoheterocyclic group or a substituted or unsubstituted C₂-C₁₀ alkyl group;

R² is hydrogen, alkyl, mercaptoalkyl, alkenyl, alkynyl, cycloalkyl, aryl, arylalkyl, thiazolyl, triazolyl, imidazolyl, benzothiazolyl, benzoimidazolyl, or linked to R¹ to form a heterocycle;

Y is SO₃⁻X⁺, OSO₃⁻X⁺, or SSO₃⁻X⁺;

X⁺ is hydrogen, a cationic group, or an ester forming moiety;

m is 0 or 1;

n is 1, 2, 3, or 4;

L is substituted or unsubstituted C₁-C₃ alkyl group or absent, or a pharmaceutically acceptable salt thereof, provided that when R¹ is alkyl, L is absent.

In a further embodiment, R² is hydrogen. In another flurther embodiment, R¹ is straight chain alkyl, for example, ethyl, n-pentyl, n-heptyl, or n-octyl. In another embodiment, R¹ is t-butyl. In yet another alternate embodiment, R¹ is C₇-C₁₀ bicycloalkyl or tricycloalkyl, such as, for example, tricyclo[3.3.1.0^{3,7}]decyl (or adamantyl), bicyclo[2.1.2]heptyl, or indolyl. In another alternate embodiment, R¹ is tetrahydronaphthyl.

In one embodiment, L² is —(CH₂)₃—. In another further embodiment, L² is —(CH₂)₄— or —(CH₂)₅—. In yet another further embodiment, L₂ is —(CH₂)₂—. In yet another further embodiment, L² is substituted alkyl, e.g., —CH₂—(CHOH)—CH₂—.

In another embodiment, L¹ is CH₂CH₂ or absent.

In a further embodiment, R¹ is branched alkyl, e.g., t-butyl. In another embodiment, R¹ is adamanyl. In another embodiment, R¹ is cyclic alkyl, e.g., cyclopropyl, cyclohexyl, cycloheptyl, cyclo-octyl, etc. The cycloalkyl moieties may be substituted further, e.g., with additional alkyl groups or other groups which allow the molecule to perform its intended function. In another embodiment, R¹ is alkyl substituted with a propargyl moiety (e.g., HC≡C—). In another embodiment, R¹ is cyclohexyl substituted with one or more methyl or propargyl groups.

In other embodiments, L¹ is a C₁-C₂ alkyl linker group (e.g., —CH(CH₃)— or —(CH₂)₂—/. In a further embodiment, R¹ is phenyl. In certain embodiments, R¹ is substituted with a methoxy group. In other embodiments, L¹ is C₃, e.g., —(CH₂)₃— or C(CH₃)₂—. In certain embodiments, L¹ is substituted, e.g., with an alkoxy, carboxylate (—COOH), benzyl , amido (—C═O—NH—), or ester (C═O—C—O) group. In certain embodiment, the ester group is a methyl, ethyl, propyl, butyl, cyclohexyl, or benzyl ester. In other embodiments, the ester group may be propenyl. In other embodiments, L¹ is substituted with a carboxylate group. In a further embodiment, R¹ is substituted with a subsituted amido group, wherein the amido group is substituted with an alkyl, e.g., methyl, ethyl, propyl, butyl, pentyl, or hexyl group. In another embodiment, the alkyl R group is a substituted with a —C═O—NH—OH, C═O—NH₂, or an amido group. In certain embodiments, the amido group is substituted with an alkyl (e.g., methyl, ethyl, propyl, butyl, pentyl, hexyl, cyclohexyl, bezyl or aryl group. In another embodiment, the amido group is substituted with a —CH(CH₂)₂ group. R¹ itself may be substituted with a phenyl or may be branched or straight chain alkyl. In certain embodiments, R¹ may also be substituted with a thioether moiety. Examples of thioethers include S—Me, S—Et, etc. In certain embodiments, the alkyl R¹ moiety is substituted with both an aryl or a thioether moiety and an amido moiety. In other embodiments, the alkyl R¹ moiety may be substituted with both a thioether and a carboxylate moiety. In other embodiments, alkyl R¹ groups are substituted with hydroxyl. R¹ groups, e.g., alkyl R¹ groups, may also be substituted with both thioether and hydroxyl groups. In other embodiments, R¹ groups, e.g., alkyl R¹ groups are substituted with cyano groups. Examples of R¹ groups including —CN moieties include —C(CH₃)₂CN, cyclohexyl substituted with one or more cyano groups, etc.

In other embodiments, alkyl R¹ groups are substituted with aryl groups. The aryl groups may be substituted phenyl, for example. The substituted phenyl may be substituted with one or more substituents such as hydroxy, cyano and alkoxy. In other embodiments, alkyl R¹ groups are substituted with tetrazolyl or substituted or unsubstituted benzyl.

In a further embodiment, L¹ is —C(CH₃)₂—(CH₂)—. In another embodiment, L¹ is —(C(CH₃)₂—CHOH—. In yet another embodiment, L¹ is —(C(CH₃)₂CH(OMe)—. In another embodiment, R¹ is substituted or unsubstituted phenyl. In a further embodiment, R¹ is para-substituted phenyl. Examples of substitutuents include but are not limited to fluorine, chlorine, bromine, iodine, methyl, t-butyl, alkoxy, methoxy, etc. In other embodiment, R¹ is substituted at the meta position. Examples of substituents include methoxy, chloro, methyl, t-butyl, fluoro, alkyl, alkoxy, iodo, trifluoralkyl, methoxy, etc. In another embodiment, R¹ is phenyl substituted in the ortho position, with similar substituents. In another embodiment, L¹ comprises a cycloalkyl moiety, e.g., cyclopentyl. In another embodiment, L¹ comprises an alkyenyl group and, optionally, a substituted aryl group, with substittuents similar to those described about.

In certain embodiments, R¹ is cyclopropyl or cyclohexyl. In certain embodiments, the cyclopropyl or cyclohexyl group is subsituted with an ether group or an alkyl group. In certain further embodiments, the ether group is a benzyl ether group.

In another embodiment, wherein R¹ is alkyl, it is substituted with groups such as phenyl, or hydroxy.

In another embodiment, the invention pertains to compounds of Formula III-D: 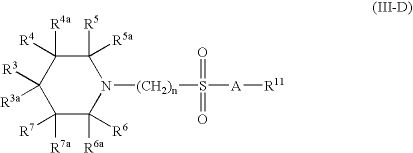
wherein:

A is nitrogen or oxygen;

R¹¹ is hydrogen, salt-forming cation, ester forming group, —(CH₂)ₓ—Q, or when A is nitrogen, A and R¹¹ taken together may be the residue of a natural or unnatural amino acid or a salt or ester thereof;

Q is hydrogen, thiazolyl, triazolyl, imidazolyl, benzothiazolyl, or benzoimidazolyl;

x is 0, 1, 2, 3, or 4;

n is 0, 1, 2, 3, 4, 5, 6, 7, 8, 9, or 10;

R³, R^{3a}, R⁴, R^{4a}, R⁵, R^{5a}, R⁶, R^{6a}, R⁷ and R^{7a} are each independently hydrogen, alkyl, mercaptoalkyl, alkenyl, alkynyl, cycloalkyl, aryl, alkylcarbonyl, arylcarbonyl, alkoxycarbonyl, cyano, halogen, amino, tetrazolyl, or two R groups on adjacent ring atoms taken together with the ring atoms form a double bond, provided that one of R³, R^{3a}, R⁴, R^{4a}, R⁵, R^{5a}, R⁶, R^{6a}, R⁷ and R^{7a} is a moiety of the Formula IIIa-D: 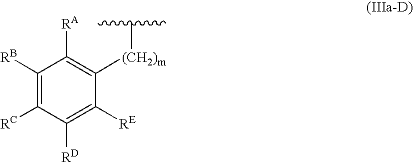
wherein:

m is 0, 1, 2, 3, or 4;

R^{A}, R^{B}, R^{C}, R^{D}, and R^{E} are independently selected from a group of hydrogen, halogen, hydroxyl, alkyl, alkoxyl, halogenated alkyl, mercaptoalkyl, alkenyl, alkynyl, cycloalkyl, aryl, cyano, thiazolyl, triazolyl, imidazolyl, tetrazolyl, benzothiazolyl, and benzoimidazolyl; and pharmaceutically acceptable salts and esters thereof, provided that said compound is not 3-(4-phenyl-1,2,3,6-tetrahydro-1-pyridyl)-1-propanesulfonic acid. In a further embodiment, n is 2, 3 or 4.

In another embodiment, R¹¹ is a salt-forming cation. Examples of salt forming cations include pharmaceutically acceptable salts described herein as well as lithium, sodium, potassium, magnesium, calcium, barium, zinc, iron, and ammonium. In another embodiment, R¹¹ is an ester-forming group. An ester-forming group includes groups when bound form an ester. Examples of such groups include substituted or unsubstituted alkyl, aryl, alkenyl, alkynyl, or cycloalkyl. In another embodiment, A is oxygen.

In another embodiment, R³ and R⁴ are taken together with the carbon atoms to which they are attached to form a double bond. In another embodiment, R^{A}, R^{B}, R^{C}, R^{D}, and R^{E} are each hydrogen. R^{A}, R^{B}, R^{D}, and R^{E} are each hydrogen and R^{C} is a halogen, such as fluorine, chlorine, iodine, or bromine.

In another embodiment, R³ or R^{5a} is a moiety of Formula IIIa-D.

In another embodiment, R⁴, R⁵, R⁶, and R⁷ are each hydrogen. In another further embodiment, R^{4a}, R^{5a}, R^{6a}, and R^{7a} are each hydrogen.

In another, R^{3a} is hydroxyl, cyano, acyl, or hydroxyl.

In another further embodiment, R¹¹ and A taken together are a natural or unnatural amino acid residue or a pharmaceutically acceptable salt or ester thereof. Examples of amino acid residues include esters and salts of phenylalanine and leucine.

In another embodiment, m is 0, 1, or 3.

In another embodiment, the invention pertains to compounds of Formula IV-D: 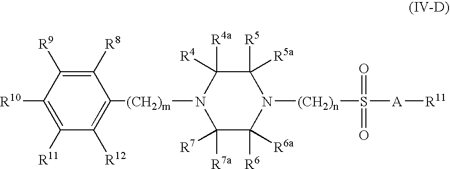
wherein:

A is nitrogen or oxygen;

R¹¹ is hydrogen, salt-forming cation, ester forming group, —(CH₂)ₓ—Q, or when A is nitrogen, A and R¹¹ taken together may be the residue of a natural or unnatural amino acid or a salt or ester thereof;

Q is hydrogen, thiazolyl, triazolyl, imidazolyl, benzothiazolyl, or benzoimidazolyl;

x is 0, 1, 2, 3, or 4;

n is 0, 1, 2, 3, 4, 5, 6, 7, 8, 9, or 10;

R⁴, R^{4a}, R⁵, R^{5a}, R⁶, R^{6a}, R⁷, and R^{7a} are each independently hydrogen, alkyl, mercaptoalkyl, alkenyl, alkynyl, cycloalkyl, aryl, alkylcarbonyl, arylcarbonyl, alkoxycarbonyl, cyano, halogen, amino, tetrazolyl, R⁴ and R⁵ taken together, with the ring atoms they are attached to, form a double bond, or R⁶ and R⁷ taken together, with the ring atoms they are attached to, form a double bond;

m is 0, 1, 2, 3, or 4;

R⁸, R⁹, R¹⁰, R¹¹, and R¹² are independently selected from a group of hydrogen, halogen, hydroxyl, alkyl, alkoxyl, halogenated alkyl, mercaptoalkyl, alkenyl, alkynyl, cycloalkyl, aryl, cyano, thiazolyl, triazolyl, imidazolyl, tetrazolyl, benzothiazolyl, and benzoimidazolyl, and pharmaceutically acceptable salts and esters thereof.

In another embodiment, R¹¹ is a salt-forming cation. Examples of salt forming cations include pharmaceutically acceptable salts described herein as well as lithium, sodium, potassium, magnesium, calcium, barium, zinc, iron, and ammonium. In another embodiment, R¹¹ is an ester-forming group. An ester-forming group includes groups when bound form an ester. Examples of such groups include substituted or unsubstituted alkyl, aryl, alkenyl, alkynyl, or cycloalkyl. In another embodiment, A is oxygen.

In another embodiment, m is 0 or 1. In another further embodiment, n is 2, 3, or 4. In another further embodiment, R⁴, R⁵, R⁶ and R⁷ are each hydrogen. R^{4a}, R^{5a}, R^{6a}, and R^{7a} also may be hydrogen. Examples of R⁸, R⁹, R¹⁰, R¹¹, and R¹² include hydrogen. In other embodiment R⁸, R⁹, R¹¹, R¹² are each hydrogen, and R¹⁰ is a halogen, (e.g., fluorine, chlorine, bromine, or iodine), nitro, or alkyl (e.g., methyl, ethyl, butyl). In another embodiment, A—R¹¹ may be the residue of an amino acid, e.g., a phenyl alanine residue. In another embodiment, R⁹, R¹⁰, R¹¹ and R¹² are each hydrogen, and R⁸ is not hydrogen, e.g., halogen, e.g., fluorine, bromine, chlorine, or iodsine.

In another embodiment, the invention pertains to compounds of Formula V-D: 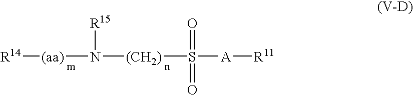
wherein:

A is nitrogen or oxygen;

R¹¹ is hydrogen, salt-forming cation, ester forming group, —(CH₂)ₓ—Q, or when A is nitrogen, A and R¹¹ taken together may be the residue of a natural or unnatural amino acid or a salt or ester thereof;

Q is hydrogen, thiazolyl, triazolyl, imidazolyl, benzothiazolyl, or benzoimidazolyl;

x is 0, 1, 2, 3, or 4;

n is 0, 1, 2, 3, 4, 5, 6, 7, 8, 9, or 10;

aa is a natural or unnatural amino acid residue;

m is 0, 1, 2, or 3;

R¹⁴ is hydrogen or protecting group;

R¹⁵ is hydrogen, alkyl or aryl, and pharmaceutically acceptable salts and prodrugs thereof.

In another embodiment, R¹¹ is a salt-forming cation. Examples of salt forming cations include pharmaceutically acceptable salts described herein as well as lithium, sodium, potassium, magnesium, calcium, barium, zinc, iron, and ammonium. In another embodiment, R¹¹ is an ester-forming group. An ester-forming group includes groups when bound form an ester. Examples of such groups include substituted or unsubstituted alkyl, aryl, alkenyl, alkynyl, or cycloalkyl. In another embodiment, A is oxygen.

In an embodiment, n is 2, 3 or 4. In certain embodiments, m is 0. In certain embodiments, A—R¹¹ is a residue of a natural amino acid, or a salt or ester thereof. Examples of amino acid residues, include, but are not limited, to leucine or phenylalanine residues, and pharmaceutically acceptable salts and esters thereof. Examples of possible esters include methyl, ethyl, and t-butyl.

In another embodiment, m is 1. Examples of aa include natural and unnatural amino acid residues such as phenylalanine, glycine, and leucine.

In another embodiment, (aa)ₘ is a residue of phe-phe; and pharmaceutically acceptable salts or an appropriate protecting group. In certain embodiments, R¹⁵ is hydrogen or substituted alkyl, e.g., arylalkyl.

The term “unnatural amino acid” refers to any derivative of a natural amino acid including D forms, and α- and β-amino acid derivatives. It is noted that certain amino acids, e.g., hydroxyproline, that are classified as a non-natural amino acid herein, may be found in nature within a certain organism or a particular protein. Amino acids with many different protecting groups appropriate for immediate use in the solid phase synthesis of peptides are commercially available. In addition to the twenty most common naturally occurring amino acids, the following examples of non-natural amino acids and amino acid derivatives may be used according to the invention (common abbreviations in parentheses): β-alanine (β-ALA), γ-aminobutyric acid (GABA), 2-aminobutyric acid (2-Abu), α,β-dehydro-2-aminobutyric acid (8-AU), 1-aminocyclopropane-1-carboxylic acid (ACPC), aminoisobutyric acid (Aib), 2-amino-thiazoline-4-carboxylic acid, 5-aminovaleric acid (5-Ava), 6-aminohexanoic acid (6-Ahx), 8-aminooctanoic acid (8-Aoc), 11-aminoundecanoic acid (11-Aun), 12-aminododecanoic acid (12-Ado), 2-aminobenzoic acid (2-Abz), 3-aminobenzoic acid (3-Abz), 4-aminobenzoic acid(4-Abz), 4-amino-3-hydroxy-6-methylheptanoic acid (Statine, Sta), aminooxyacetic acid (Aoa), 2-aminotetraline-2-carboxylic acid (ATC), 4-amino-5-cyclohexyl-3-hydroxypentanoic acid (ACHPA), para-aminophenylalanine (4-NH₂-Phe), biphenylalanine (Bip), para-bromophenylalanine (4-Br-Phe), ortho-chlorophenylalanine] (2-Cl-Phe), meta-chlorophenylalanine (3-Cl-Phe), para-chlorophenylalanine (4-Cl-Phe), meta-chlorotyrosine (3-Cl-Tyr), para-benzoylphenylalanine (Bpa), tert-butylglycine (TLG), cyclohexylalanine (Cha), cyclohexylglycine (Chg), 2,3-diaminopropionic acid (Dpr), 2,4-diaminobutyric acid (Dbu), 3,4-dichlorophenylalanine (3,4-Cl₂-Phe), 3,4-diflurorphenylalanine (3,4-F₂-Phe), 3,5-diiodotyrosine (3,5-I₂-Tyr), ortho-fluorophenylalanine (2-F-Phe), meta-fluorophenylalanine (3-F-Phe), para-fluorophenylalanine (4-F-Phe), meta-fluorotyrosine (3-F-Tyr), homoserine (Hse), homophenylalanine (Hfe), homotyrosine (Htyr), 5-hydroxytryptophan (5-OH-Trp), hydroxyproline (Hyp), para-iodophenylalanine (4-1-Phe), 3-iodotyrosine (3-1-Tyr), indoline-2-carboxylic acid (Idc), isonipecotic acid (Inp), meta-methyltyrosine (3-Me-Tyr), 1-naphthylalanine (1-Nal), 2-naphthylalanine (2-Nal), para-nitrophenylalanine (4-NO₂-Phe), 3-nitrotyrosine (3-NO₂-Tyr), norleucine (Nle), norvaline (Nva), ornithine (Orn), ortho-phosphotyrosine (H₂PO₃-Tyr), octahydroindole-2-carboxylic acid (Oic), penicillamine (Pen), pentafluorophenylalanine (F₅-Phe), phenylglycine (Phg), pipecolic acid (Pip), propargylglycine (Pra), pyroglutamic acid (PGLU), sarcosine (Sar), tetrahydroisoquinoline-3-carboxylic acid (Tic), and thiazolidine-4-carboxylic acid (thioproline, Th). Additionally, N-alkylatd amino acids may be used, as well as amino acids having amine-containing side chains (such as Lys and Orn) in which the amine has been acylated or or alkylated.

In another embodiment, the invention pertains, at least in part, to compounds of Formula VI-D: 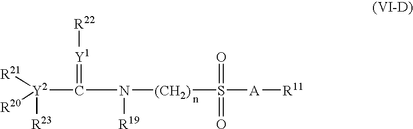
wherein:

n is 1, 2, 3, 4, 5, 6, 7, 8, 9, or 10;

A is oxygen or nitrogen;

R¹¹ is hydrogen, salt-forming cation, ester forming group, —CH₂)ₓ—Q, or when A is nitrogen, A and R¹¹ taken together may be the residue of a natural or unnatural amino acid or a salt or ester thereof;

Q is hydrogen, thiazolyl, triazolyl, imidazolyl, benzothiazolyl, or benzoimidazolyl;

xis 0, 1, 2, 3, or 4;

R¹⁹ is hydrogen, alkyl or aryl;

Y¹ is oxygen, sulfur, or nitrogen;

Y² is carbon, nitrogen, or oxygen;

R²⁰ is hydrogen, alkyl, amino, mercaptoalkyl, alkenyl, alkynyl, cycloalkyl, aryl, arylalkyl, thiazolyl, triazolyl, tetrazolyl, imidazolyl, benzothiazolyl, or benzoimidazolyl;

R²¹ is hydrogen, alkyl, mercaptoalkyl, alkenyl, alkynyl, cycloalkyl, aryl, arylalkyl, thiazolyl, triazolyl, tetrazolyl, imidazolyl, benzothiazolyl, benzoimidazolyl, or absent if Y² is oxygen;

R²² is hydrogen, alkyl, mercaptoalkyl, alkenyl, alkynyl, cycloalkyl, aryl, arylalkyl, thiazolyl, triazolyl, tetrazolyl, imidazolyl, benzothiazolyl, benzoimnidazolyl; or R²² is hydrogen, hydroxyl, alkoxy or aryloxy if Y¹ is nitrogen; or R²² is absent if Y¹ is oxygen or sulfur; or R²² and R²¹ may be linked to form a cyclic moiety if Y¹ is nitrogen;

R²³ is hydrogen, alkyl, amino, mercaptoalkyl, alkenyl, alkynyl, cycloalkyl, aryl, arylalkyl, thiazolyl, triazolyl, tetrazolyl, imidazolyl, benzothiazolyl, or benzoimidazolyl, or absent if Y² is nitrogen or oxygen;

or pharmaceutically acceptable salts thereof.

In another embodiment, R¹¹ is a salt-forming cation. Examples of salt forming cations include pharmaceutically acceptable salts described herein as well as lithium, sodium, potassium, magnesium, calcium, barium, zinc, iron, and ammonium. In a further embodiment, the salt is a sodium salt. In a further, embodiment, A is oxygen.

In another embodiment, Y¹ is oxygen or sulfur, and R²² is absent.

In another embodiment, Y² is oxygen and R²¹ is absent. Examples of R²⁰ include benzyl, aryl (e.g., phenyl), alkyl, cycloalkyl (e.g., adamantyl), etc. In other embodiment, Y² is nitrogen and R²¹ is hydrogen. In other embodiment, R²¹ is benzyl. In another fuirther embodiment, R²⁰ and R²¹ are linked to form a pyridyl ring. In another embodiment, Y¹ is sulfur.

In another embodiment, the invention pertains to compounds of Formula VII-D: 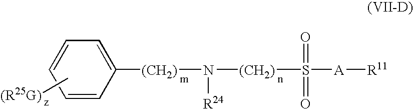
wherein:

n is 2, 3,or 4;

A is oxygen or nitrogen;

R¹¹ is hydrogen, salt-forming cation, ester forming group, —(CH₂)ₓ—Q, or when A is nitrogen, A and R¹¹ taken together may be the residue of a natural or unnatural amino acid or a salt or ester thereof;

Q is hydrogen, thiazolyl, triazolyl, imidazolyl, benzothiazolyl, or benzoimidazolyl;

x is 0, 1, 2, 3, or 4;

G is a direct bond or oxygen, nitrogen, or sulfur;

z is 0, 1, 2, 3, 4, or 5;

m is 0 or 1;

R²⁴ is selected from a group consisting of hydrogen, alkyl, mercaptoalkyl, alkenyl, alkyriyl, aroyl, alkylcarbonyl, aminoalkylcarbonyl, cycloalkyl, aryl, arylalkyl, thiazolyl, triazolyl, imidazolyl, benzothiazolyl, and benzoimidazolyl;

each R²⁵ is independently selected from hydrogen, halogen, cyano, hydroxyl, alkoxy, thiol, amino, nitro, alkyl, aryl, carbocyclic, or heterocyclic, and pharmaceutically acceptable salts thereof.

In one embodiment, R¹¹ is hydrogen. In another, A is oxygen. For example, n may be 3 and m may be 1. In other embodiments, R²⁴ is hydrogen or benzyl.

In certain embodiments, z is 0, 2, or 3. In others, R²⁵ is hydroxyl or alkoxy, e.g., methoxy, ethoxy, etc. In certain embodiments, two or more R²⁵ substituents can be linked to form a fused ring (e.g., to form a methylendioxyphenyl moiety).

The invention pertains to both salt forms and acid/base forms of the compounds of the invention. For example, the invention pertains not only to the particular salt forms of compounds shown herein as salts, but also the invention includes other pharmaceutically acceptable salts, and the acid and/or base form of the compound. The invention also pertains to salt forms of compounds shown herein.

Exemplary compounds of the invention are also shown in the Figures attached hereto. Intended to be part of this invention are the exemplary compounds and selected groups and subsets thereof for any of the formulas recited herein, e.g., Formula I-D through VII-D, provided the two U.S. Patent Applications filed Jun. 18, 2004, both entitled “Methods and Compositions for Treating Amyloid Related Diseases” (Attorney Docket Nos. NBI-162A and NBI-162B), and the U.S. Patent Application filed Jun. 18, 2004, entitled “Methods and Compositions for the Treatment of Amyloid- and Epileptogenesis-Associated Diseases” (Attorney Docket Nos. NBI-163), which are expressly incorporated by reference herein.

In one embodiment, the invention does not pertain to the compounds described in WO 00/64420, WO 97/023458 and WO 96/28187. In one embodiment, the invention does not pertain to methods of using the compounds described in WO 00/64420, WO 97/023458 and WO 96/28187 for the treatment of diseases or disorders described therein. In a further embodiment, the invention pertains to methods of using the compounds described in WO 00/64420, WO 97/023458 and WO 96/28187 for methods described in this application, which are not described in WO 00/64420, WO 97/023458 and WO 96/28187. Moreover WO 00/64420, WO 97/023458 and WO 96/28187 are incorporated by reference herein in their entirety.

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here. Functional and structural equivalents of the compounds described herein and which have the same general properties, wherein one or more simple variations of substituents are made which do not adversely affect the essential nature or the utility of the compound. The compounds of the present invention may be readily prepared in accordance with the synthesis schemes and protocols described herein, as illustrated in the specific procedures provided. However, those skilled in the art will recognize that other synthetic pathways for forming the compounds of this invention may be used, and that the following is provided merely by way of example, and is not limiting to the present invention. See, e.g., “Comprehensive Organic Transformations” by R. Larock, VCH Publishers (1989). It will be further recognized that various protecting and deprotecting strategies will be employed that are standard in the art (See, e.g., “Protective Groups in Organic Synthesis” by Greene and Wuts). Those skilled in the relevant arts will recognize that the selection of any particular protecting group (e.g., amine and carboxyl protecting groups) will depend on the stability of the protected moiety with regards to the subsequent reaction conditions and will understand the appropriate selections. Further illustrating the knowledge of those skilled in the art is the following sampling of the extensive chemical literature: “Chemistry of the Amino Acids” by J. P. Greenstein and M. Winitz, John Wiley & Sons, Inc., New York (1961); “Comprehensive Organic Transformations” by R. Larock, VCH Publishers (1989); T. D. Ocain, et al., J. Med. Chem. 31, 2193-99 (1988); E. M. Gordon, et al., J. Med. Chem. 31, 2199-10 (1988); “Practice of Peptide Synthesis” by M. Bodansky and A. Bodanszky, Springer-Verlag, New York (1984); “Protective Groups in Organic Synthesis” by T. Greene and P. Wuts (1991); “Asymmetric Synthesis: Construction of Chiral Molecules Using Amino Acids” by G. M. Coppola and H. F. Schuster, John Wiley & Sons, Inc., New York (1987); “The Chemical Synthesis of Peptides” by J. Jones, Oxford University Press, New York (1991); and “Introduction of Peptide Chemistry” by P. D. Bailey, John Wiley & Sons, Inc., New York (1992).

The chemical structures herein are drawn according to the conventional standards known in the art. Thus, where an atom, such as a carbon atom, as drawn appears to have an unsatisfied valency, then that valency is assumed to be satisfied by a hydrogen atom even though that hydrogen atom is not necessarily explicitly drawn. The structures of some of the compounds of this invention include stereogenic carbon atoms. It is to be understood that isomers arising from such asymmetry (e.g., all enantiomers and diastereomers) are included within the scope of this invention unless indicated otherwise. That is, unless otherwise stipulated, any chiral carbon center may be of either (R)- or (S)-stereochemistry. Such isomers can be obtained in substantially pure form by classical separation techniques and by stereochemically-controlled synthesis. Furthermore, alkenes can include either the E- or Z-geometry, where appropriate. In addition, the compounds of the present invention may exist in unsolvated as well as solvated forms with acceptable solvents such as water, THF, ethanol, and the like. In general, the solvated forms are considered equivalent to the unsolvated forms for the purposes of the present invention.

It should be understood that the use of any of the compounds described herein or in the applications identified in “The Related Applications” Section are within the scope of the present invention and are intended to be encompassed by the present invention and are expressly incorporated herein at least for these purposes, and are furthermore expressly incorporated for all other purposes.

In one embodiment, the Protein Aggregation Disorder may be Pick's Disease, corticobasal degeneration, progressive supranuclear palsy, amyotrophic lateral sclerosis/parkinsonism dementia complex, Parkinson's Disease (PD), Huntington's disease (HD), dystrophia myotonica, dentatorubro-pallidoluysian atrophy, Friedreich's ataxia, fragile X syndrome, fragile XE mental retardation, spinobulbar muscular atrophy, Wilson's Disease, and spinocerebellar ataxia type 1 (SCA1); spinocerebellar ataxia type 2 (SCA2), Machado-Joseph disease (MJD or SCA3), spinocerebellar ataxia type 6 (SCA6), spinocerebellar ataxia type 7 (SCA7), spinocerebellar ataxia type 17 (SCA17), chronic liver diseases, cataracts, serpinopathies, haemolytic anemia, cystic fibrosis, neurofibromatosis type 2, demyelinating peripheral neuropathies, retinitis pigmentosa, Marfan syndrome, emphysema, idiopathic pulmonary fibrosis, Argyophilic grain dementia, corticobasal degeneration, diffuse neurofibrillary tangles with calcification, frontotemporal dementia/parkinsonism linked to chromosome 17, Hallervorden-Spatz disease, Nieman-Pick disease type C, or subacute sclerosing panencephalitis.

In one embodiment, the Protein Aggregation Disorder is an Alpha-Synucleinopathy. In a preferred embodiment, the Protein Aggregation Disorder is Parkinson's Disease, Shy-Drager syndrome, Neurologic orthostatic hypotension, Shy-McGee-Drager syndrome, or Parkinson's plus syndrome.

In another embodiment, the Protein Aggregation Disorder is a Tauopathy provided that the Tauopathy is not Alzheimer's disease, Prion diseases, or cerebral amyloid angiopathy.

In a preferred embodiment the Taupathy is Amyotrophic lateral sclerosis/parkinsonism-dementia complex, Argyophilic grain dementia, DLBD, Corticobasal degeneration, Diffuse neurofibrillary tangles with calcification, Frontotemporal dementia/parkinsonism linked to chromosone-17, Hallervorden-Spatz disease, Multiple system atrophy, Nieman-Pick disease type C, Pick's disease, Progressive supranuclear palsy or Subacute sclerosing panencephalitis.

The present invention relates to a method of treating, preventing or modulating a Protein Aggregation Disorder or Proteopathy that is not an Amyloid Proteopathy. One aspect of the invention relates to a method for treating, preventing or modulating a Protein Aggregation Disorder or Proteopathy due to a familial mutation in a gene sequence, such as, for example, the following non-limiting examples: familial Parkinson's Disease (in which, for example, α-synuclein, parkin and COOH-terminal hydrolase L1 may form detrimental protein aggregates); dystrophia myotonica (e.g., in which detrimental protein aggregates of dystrophia myotonica protein kinase (DMPK) form); dentatorubro-pallidoluysian atrophy (DRPLA) (e.g., in which detrimental protein aggregation of the DRPLA gene occur); Friedreich's ataxia (e.g., in which detrimental protein aggregates of the Frataxin (FRDA) gene form); mutations in the androgen receptor (AR) (in which, e.g., detrimental protein aggregates in spinobulbar muscular atrophy form) (also known as Kennedy's Disease); spinocerebellar ataxia caused by, e.g., mutations in the SCA1 gene; Huntington's disease (HD) caused, e.g., by mutations in the huntingtin or IT15 gene leading to the formation of detrimental protein aggregates; Huntington's disease-like disorder, caused e.g. by mutations in the junctophilin-3 (JPH3/HDL2) or TBP gene; familial encephalopathy with neuroserpin inclusion bodies (FENIB) caused e.g., by mutations in the neuroserpin gene leading to detrimental protein aggregates; or Amyotrophic Lateral Sclerosis (ALS) in which, e.g., mutations in the SOD1 gene lead to detrimental protein aggregates; wherein said disorder is not an Amyloid Proteopathy.

In one embodiment of the invention, the accumulation or oligomerization of a detrimental protein aggregate may be associated with an idiopathic mutation in a gene sequence, or occur sporadically as in the non-limiting example of amyotrophic lateral sclerosis/parkinsonism dementia complex in which tau aggregates to form detrimental protein aggregates. One aspect of the invention relates to a method for treating, preventing or modulating a Protein Aggregation Disorder or Proteopathy that occurs idiopathically, such as, for example, the following: Parkinson's Disease (PD), diffuse Lewy body dementia (DLBD), multiple system atrophy (MSA), dystrophia myotonica, dentatorubro-pallidoluysian atrophy (DRPLA), Friedreich's ataxia, fragile X syndrome, fragile XE mental retardation, Machado-Joseph Disease, spinobulbar muscular atrophy (also known as Kennedy's Disease), spinocerebellar ataxia, Huntington's disease (HD), familial encephalopathy with neuroserpin inclusion bodies (FENIB), Pick's disease, corticobasal degeneration (CBD), progressive supranuclear palsy (PSP), amyotrophic lateral sclerosis/parkinsonism dementia complex, Amyotrophic Lateral Sclerosis (ALS), Cataract, or Wilson's Disease, wherein said disorder is not an Amyloid Proteopathy.

Also, the invention relates to a method of modulating a detrimental protein aggregate, provided it is not associated with an amyloid proteopathy as defined herein, comprising contacting a detrimental protein aggregate with an effective amount of the compound of the invention, such that the detrimental protein aggregate is modulated.

In one embodiment of the invention, the detrimental protein aggregate is associated with a misfolding of mature protein.

In another embodiment, the detrimental protein aggregate is extracellular.

In a further embodiment, the detrimental protein aggregate is intracellular.

In another embodiment, the detrimental protein aggregate is cytosolic.

In one embodiment, the detrimental protein aggregate is nuclear.

In another embodiment, the detrimental protein aggregate is intra-membranal.

In another embodiement, the detrimental protein aggregate is associated with an aggresome.

In a further embodiment, the detrimental protein aggregate is associated with improper degradation of protein.

In another embodiment, the detrimental protein aggregate is in the endoplasmic reticulum.

In another embodiment, the detrimental protein aggregate is in the trans-Golgi network.

In one embodiment, the detrimental protein aggregate is associated with misfolded protein that has evaded the ubiquitin-proteasome system.

In another embodiment, the detrimental protein aggregate is modulated by enhancing degradation of said protein aggregate.

In another embodiment, the detrimental protein aggregate is inhibited.

In a further embodiment, the detrimental protein aggregate is modulated by increasing the clearance of the protein aggregate.

In another embodiment the detrimental protein aggregate is associated with improper posttranscriptional modification, improper posttranslational modification, misfolding of mature protein due to, for example, lack of an appropriate chaperone molecule, improper degradation of protein, evasion by a protein of the ubiquitin-proteasome system, which results in the formation of inclusion bodies, aggregates, aggresomes, precipitated protein, insoluble aggregates, or any combination thereof.

In another embodiment the detrimental protein aggregate is associated with fibrils, β-sheets, or hydrophobic domains.

As used herein, “treatment” of a subject includes the application or administration of a therapeutic compound to a subject, or application or administration of a therapeutic compound to a cell or tissue from a subject, having a disease or disorder, a symptom of a disease or disorder, or at risk of (or susceptible to) a disease or disorder, with the purpose of curing, healing, alleviating, relieving, altering, remedying, ameliorating, improving, or affecting the disease or disorder, the symptom of the disease or disorder, or the risk of (or susceptibility to) the disease or disorder.

The term “subject” includes living organisms in which Protein Aggregation Disorders can occur. Examples of subjects include humans, monkeys, cows, sheep, goats, dogs, cats, mice, rats, and transgenic species thereof. Administration of the compositions of the present invention to a subject to be treated can be carried out using known procedures, at dosages and for periods of time effective to modulate detrimental protein aggregation in the subject as further described herein. An “effective amount” of the therapeutic compound necessary to achieve a therapeutic effect may vary according to factors such as the amount of protein already deposited at the clinical site in the subject, the age, sex, and weight of the subject, and the ability of the therapeutic compound to modulate detrimental protein aggregation in the subject. Dosage regimens can be adjusted to provide the optimum therapeutic response. For example, several divided doses may be administered daily or the dose may be proportionally reduced as indicated by the exigencies of the therapeutic situation.

In certain embodiments of the invention, the subject is in need of treatment by the methods of the invention, and is selected for treatment based on this need. A subject in need of treatment is art-recognized, and includes subjects that have been identified as having a disease or disorder related to a Protein Aggregation Disorder, having a symptom of such a disease or disorder, or at risk of such a disease or disorder, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder).

The term “modulating” is intended to encompass prevention or stopping of detrimental protein aggregation or accumulation, inhibition or slowing down of further detrimental protein aggregation in a subject with ongoing Protein Aggregation Disorder, e.g., already having protein aggregates, and reducing, reversing or facilitating clearance of detrimental protein aggregates in a subject with ongoing Protein Aggregation Disorder. Modulation of detrimental protein aggregation is determined relative to an untreated subject or relative to the treated subject prior to treatment, or, e.g., determined by clinically measurable improvement, such as, in the case of a patient with e.g., Parkinson's disease or a synucleinopathy, stabilization of cognitive function or prevention of a further decrease in cognitive function (i.e., preventing, slowing, or stopping disease progression). The term “modulating” is intended to encompass both inhibition, as defined below, and enhancement of detrimental protein aggregation. The term “modulating” is intended, therefore, to encompass 1) prevention or stopping of detrimental protein aggregation or accumulation, inhibition or slowing down of further detrimental protein aggregation in a subject with ongoing Protein Aggregation Disorder, e.g., already having detrimental protein aggregation, and reducing or reversing of detrimental protein aggregation in a subject with ongoing Protein Aggregation Disorder, and 2) enhancing detrimental protein aggregation, e.g., increasing the rate or amount of detrimental protein aggregation in vivo or in vitro. Detrimental protein aggregation-enhancing compounds may be useful in animal models of Protein Aggregation Disorder, for example, to make possible the development of detrimental protein aggregation in animals in a shorter period of time or to increase detrimental protein aggregation over a selected period of time. Detrimental protein aggregation-enhancing compounds may be useful in screening assays for compounds which inhibit detrimental protein aggregation in vivo, for example, in animal models, cellular assays and in vitro assays for detrimental protein aggregation. Such compounds may be used, for example, to provide faster or more sensitive assays for compounds. In some cases, detrimental protein aggregation enhancing compounds may also be administered for therapeutic purposes, e.g., to enhance the deposition of detrimental protein aggregation. Modulation of detrimental protein aggregation is determined relative to an untreated subject or relative to the treated subject prior to treatment.

“Inhibition” of detrimental protein aggregation includes preventing or stopping of aggresome formation, inhibiting or slowing down of further amyloid deposition in a subject with amyloidosis, e.g., already having protein aggregates, and reducing or reversing Protein Aggregation Disorders or deposits in a subject with ongoing Protein Aggregation Disorder. Inhibition of detrimental protein aggregation is determined relative to an untreated subject, or relative to the treated subject prior to treatment, or, e.g., determined by clinically measurable improvement, such as, in the case of a patient with e.g., Parkinson's disease or a synucleinopathy, stabilization of cognitive function or prevention of a further decrease in cognitive function (i.e., preventing, slowing, or stopping disease progression).

In accordance with the present invention, there is further provided a method for modulating cellular toxicity, preferably neurotoxicity, associated with a detrimental protein aggregate comprising contacting a detrimental protein aggregate with an effective amount of the compound of the invention, such that cellular toxicity is modulated. In a preferred embodiment, the cellular toxicity is associated with inclusions. In another preferred embodiment, cellular toxicity is modulated in a neuronal cell or a glial cell.

The present invention also provides a method for treating or preventing a Neuofibrillary Tangle associated with tau, in a subject (preferably a mammal, more preferably a human) comprising administering to the subject an effective amount of a compound of the invention, such that Neurofibrillary Tangle associated with tau is treated or prevented. In another embodiment, the invention relates to a method of modulating a Neurofibrillary Tangle associated with tau, in a subject (preferably a mammal, more preferably a human) comprising administering to a subject an effective amount of a compound of the invention, such that a Neurofibrillary Tangle associated with tau is modulated.

The present invention provides a method for treating or preventing an inclusion containing α-synuclein NAC fragment, in a subject (preferably a mammal, more preferably a human) comprising administering to the subject an effective amount of a compound of the invention, such that an inclusion containing α-synuclein NAC fragment is treated or prevented. In another embodiment, the invention relates to a method of modulating an inclusion containing α-synuclein NAC fragment, in a subject (preferably a mammal, more preferably a human) comprising administering to a subject an effective amount of a compound of the invention, such that an inclusion containing α-synuclein NAC fragment is modulated.

In another embodiment the detrimental protein aggregate is associated with one of the following proteins or fragments: α-synuclein, tau, NAC, huntingtin, DRPLA, Schwannomin, cytokeratin, myelin protein 22, rhodopsin, atrophin-1, fibrillin-1, ataxin-1, ataxin-2, ataxin-3, ataxin-6, ataxin-17, androgen receptor, surfactant protein-C or alpha1-antitrypsin.

The compounds of the invention may be administered therapeutically or prophylactically to treat diseases associated with detrimental protein aggregation formation, development of detrimental protein aggregates into aggresomes, or deposition of detrimental protein aggregates into inclusion bodies, such as Lewy bodies. The compounds of the invention may bind to the form of the protein that forms the aggregate prior to its inclusion in a protein aggregate or once it is part of an aggregate, or it may bind to the aggregate itself. The compounds of the invention may also bind to the native form of the protein and prevent its conformational change into the form that forms the detrimental aggregate. The compounds of the invention may act to ameliorate the course of Protein Aggregation Disorders using any of the following mechanisms (this list is meant to be illustrative and not limiting): slowing the rate of detrimental protein aggregate formation or deposition; lessening the degree of detrimental protein aggregate deposition; inhibiting, reducing, or preventing detrimental protein aggregate fibril formation; inhibiting neurodegeneration or cellular toxicity induced by detrimental protein aggregation; inhibiting detrimental protein aggregate induced inflammation; or enhancing the clearance of detrimental protein aggregate from the brain.

The compounds of the invention may be used alone or in combination with a second compound. The compound may be any compound or substance known in the art which may be beneficial to the subject. The second compound may be any compound which is known in the art to treat, prevent, or reduce the symptoms of a Protein Aggregation Disorder, e.g. Parkinson's Disease. Furthermore, the second compound may be any compound of benefit to the subject when administered in combination with the administration of a compound of the invention, e.g. a neuroprotective compound. The language “in combination with” a second compound includes co-administration of the compounds of the invention, and with the second compound, administration of the compound of the invention first, followed by the second compound and administration of the second compound first, followed by the compound of the invention.

Compounds of the invention may be effective in controlling detrimental protein aggregate deposition either following their entry into the brain (following penetration of the blood brain barrier) or from the periphery. When acting from the periphery, a compound may alter the equilibrium of, for example, α-synuclein, between the brain and the plasma so as to favor the exit of α-synuclein from the brain. An increase in the exit of α-synuclein from the brain would result in a decrease in α-synuclein brain concentration and therefore favor a decrease in α-synuclein deposition in aggregates or Lewy bodies. Alternatively, compounds that penetrate the brain could control deposition by acting directly on brain α-synuclein e.g., by maintaining it in a non-fibrillar form or favoring its clearance from the brain.

Subjects and Patient Populations

The term “subject” includes living organisms in which amyloidosis can occur, or which are susceptible to a Protein Aggregation Disorder, as described herein. Examples of subjects include humans, chickens, ducks, peking ducks, geese, monkeys, cows, rabbits, sheep, goats, dogs, cats, mice, rats, and transgenic species thereof. Administration of the compositions of the present invention to a subject to be treated can be carried out using known procedures, at dosages and for periods of time effective to modulate detrimental protein aggregation or aggregate-induced toxicity in the subject as further described herein. An effective amount of the therapeutic compound necessary to achieve a therapeutic effect may vary according to factors such as the amount of detrimental protein aggregate already deposited at the clinical site in the subject, the age, sex, and weight of the subject, and the ability of the therapeutic compound to modulate detrimental protein aggregation in the subject. Dosage regimens can be adjusted to provide the optimum therapeutic response. For example, several divided doses may be administered daily or the dose may be proportionally reduced as indicated by the exigencies of the therapeutic situation.

In an exemplary aspect of the invention, the subject is a human. For example, the subject may be a human over 30 years old, a human over 40 years old, a human over 50 years old, a human over 60 years old, a human over 70 years old, a human over 80 years old, a human over 85 years old, a human over 90 years old, or a human over 95 years old. The subject may be a female human, including a postmenopausal female human, who may be on hormone (estrogen) replacement therapy. The subject may also be a male human. In another embodiment, the subject is under 40 years old.

A subject may be a human at risk for a Protein Aggregation Disorder, e.g., being over the age of 40 or having a predisposition for a Protein Aggregation Disorder. Protein Aggregation Disorder predisposing factors identified or proposed in the scientific literature include, among others, a genotype predisposing a subject to a Protein Aggregation Disorder; environmental factors predisposing a subject to a Protein Aggregation Disorder; past history of infection by viral and bacterial agents predisposing a subject to a Protein Aggregation Disorder; and vascular factors predisposing a subject to a Protein Aggregation Disorder. A subject may also have one or more risk factors for cardiovascular disease (e.g., atherosclerosis of the coronary arteries, angina pectoris, and myocardial infarction) or cerebrovascular disease (e.g., atherosclerosis of the intracranial or extracranial arteries, stroke, syncope, and transient ischemic attacks), such as hypercholesterolemia, hypertension, diabetes, cigarette smoking, familial or previous history of coronary artery disease, cerebrovascular disease, and cardiovascular disease. Hypercholesterolemia typically is defined as a serum total cholesterol concentration of greater than about 5.2 mmol/L (about 200 mg/dL).

The methods of the present invention can be used for one or more of the following: to prevent a Protein Aggregation Disorder, to treat a Protein Aggregation Disorder, or to ameliorate symptoms of a Protein Aggregation Disorder, or to regulate production of detrimental protein aggregates. In one embodiment, the human has a family history of a Protein Aggregation Disorder or a dementia illness.

In another embodiment, the human is at least about 40 years of age. In another embodiment, the human is at least about 60 years of age. In another embodiment, the human is at least about 70 years of age. In another embodiment, the human is at least about 80 years of age. In another embodiment, the human is at least about 85 years of age. In one embodiment, the human is between about 60 and about 100 years of age.

In still a further embodiment, the subject is shown to be at risk by a diagnostic brain imaging technique, for example, one that measures brain activity, plaque deposition, e.g., detrimental protein aggregation, or brain atrophy.

In still a further embodiment, the subject is shown to be at risk by a cognitive test such as Clinical Dementia Rating (“CDR”) or Mini-Mental State Examination (“MMSE”). The subject may exhibit a below average score on a cognitive test, as compared to a historical control of similar age and educational background. The subject may also exhibit a reduction in score as compared to previous scores of the subject on the same or similar cognition tests.

In determining the CDR, a subject is typically assessed and rated in each of six cognitive and behavioural categories: memory, orientation, judgement and problem solving, community affairs, home and hobbies, and personal care. The assessment may include historical information provided by the subject, or preferably, a corroborator who knows the subject well. The subject is assessed andrated in each of these areas and the overall rating, (0, 0.5, 1.0, 2.0 or 3.0) determined. A rating of 0 is considered normal. A rating of 1.0 is considered to correspond to mild dementia. A subject with a CDR of 0.5 is characterized by mild consistent forgetfulness, partial recollection of events and “benign” forgetfulness. In one embodiment the subject is assessed with a rating on the CDR of above 0, of above about 0.5, of above about 1.0, of above about 1.5, of above about 2.0, of above about 2.5, or at about 3.0.

Another test is the Mini-Mental State Examination (MMSE), as described by Folstein “Mini-mental state. A practical method for grading the cognitive state of patients for the clinician.” J. Psychiatr. Res. 12:189-198, 1975. The MMSE evaluates the presence of global intellectual deterioration. See also Folstein “Differential diagnosis of dementia. The clinical process.” Psychiatr Clin North Am. 20:45-57, 1997. The MMSE is a means to evaluate the onset of dementia and the presence of global intellectual deterioration. The MMSE is scored from 1 to 30. The MMSE does not evaluate basic cognitive potential, as, for example, the so-called IQ test. Instead, it tests intellectual skills. A person of “normal” intellectual capabilities will score a “30” on the MMSE objective test (however, a person with a MMSE score of 30 could also score well below “normal” on an IQ test). See, e.g., Kaufer, J. Neuropsychiatry Clin. Neurosci. 10:55-63, 1998; Becke, Alzheimer Dis Assoc Disord. 12:54-57, 1998; Ellis, Arch. Neurol. 55:360-365, 1998; Magni, Int. Psychogeriatr. 8:127-134, 1996; Monsch, Acta Neurol. Scand. 92:145-150, 1995. In one embodiment, the subject scores below 30 at least once on the MMSE. In another embodiment, the subject scores below about 28, below about 26, below about 24, below about 22, below about 20, below about 18, below about 16, below about 14, below about 12, below about 10, below about 8, below about 6, below about 4, below about 2, or below about 1.

In another embodiment, the subject exhibits no symptoms of a Protein Aggregation Disorder. In another embodiment, the subject is a human who is at least 40 years of age and exhibits no symptoms of a Protein Aggregation Disorder. In another embodiment, the subject is a human who is at least 40 years of age and exhibits one or more symptoms of a Protein Aggregation Disorder.

The methods of the invention may be applied as a therapy for a subject having a Protein Aggregation Disorder, or the methods of the invention may be applied as a prophylaxis against a Protein Aggregation Disorder or dementia for a subject with such a predisposition, as in a subject, e.g., with a genomic mutation

It is to be understood that wherever values and ranges are provided herein, e.g., in ages of subject populations, dosages, and blood levels, all values and ranges encompassed by these values and ranges, are meant to be encompassed within the scope of the present invention. Moreover, all values in between these values and ranges may also be the upper or lower limits of a range.

Pharmaceutical Preparations

In another embodiment, the present invention relates to pharmaceutical compositions comprising compounds according to any of the Formulae herein for the treatment of an Protein Aggregation Disorder, as well as methods of manufacturing such pharmaceutical compositions.

In general, the compounds of the present invention may be prepared by the methods illustrated in the general reaction schemes as, for example, in the patents and patent applications refered to herein, or by modifications thereof, using readily available starting materials, reagents and conventional synthesis procedures. In these reactions, it is also possible to make use of variants which are in themselves known, but are not mentioned here. Functional and structural equivalents of the agents described herein and which have the same general properties, wherein one or more simple variations of substituents are made which do not adversely affect the essential nature or the utility of the agent are also included.

The agents of the invention may be supplied in a solution with an appropriate solvent or in a solvent-free form (e.g., lyophilized). In another aspect of the invention, the agents and buffers necessary for carrying out the methods of the invention may be packaged as a kit, optionally including a container. The kit may be commercially used according to the methods described herein and may include instructions for use in a method of the invention. Additional kit components may include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

The term “container” includes any receptacle for holding the therapeutic formulation. For example, in one embodiment, the container is the packaging that contains the formulation. In other embodiments, the container is not the packaging that contains the formulation, i.e., the container is a receptacle, such as a box or vial that contains the packaged formulation or unpackaged formulation and the instructions for use of the formulation. Moreover, packaging techniques are well known in the art. It should be understood that the instructions for use of the therapeutic formulation may be contained on the packaging containing the therapeutic formulation, and as such the instructions form an increased finctional relationship to the packaged product.

The therapeutic compound may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

To administer the therapeutic compound by other than parenteral administration, it may be necessary to coat the compound with, or co-administer the compound with, a material to prevent its inactivation. For example, the therapeutic compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., (1984) J. Neuroimmunol. 7:27).

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fuigi.

The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents are included, for example, sugars, sodium chloride, or polyalcohols such as mannitol and sorbitol, in the composition. Prolonged absorption of the injectable compositions can be brought about by including in the composition a compound which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile vehicle which contains a basic dispersion medium and the required other ingredients from those enumerated above. In the case of sterile powders for the preparation of sterile injectable solutions, the methods of preparation are vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of agenting such a therapeutic compound for the treatment of amyloid deposition in subjects.

The present invention therefore includes pharmaceutical formulations comprising the agents of the Formulae described herein, including pharmaceutically acceptable salts thereof, in pharmaceutically acceptable carriers for aerosol, oral and parenteral administration. Also, the present invention includes such agents, or salts thereof, which have been lyophilized and which may be reconstituted to form pharmaceutically acceptable formulations for administration, as by intravenous, intramuscular, or subcutaneous injection. Administration may also be intradermal or transdermal.

In accordance with the present invention, a compound of the Formulae described herein, and pharmaceutically acceptable salts thereof, may be administered orally or through inhalation as a solid, or may be administered intramuscularly or intravenously as a solution, suspension or emulsion. Alternatively, the agents or salts may also be administered by inhalation, intravenously or intramuscularly as a liposomal suspension.

Pharmaceutical formulations are also provided which are suitable for administration as an aerosol, by inhalation. These formulations comprise a solution or suspension of the desired compound of any Formula herein, or a salt thereof, or a plurality of solid particles of the compound or salt. The desired formulation may be placed in a small chamber and nebulized. Nebulization may be accomplished by compressed air or by ultrasonic energy to form a plurality of liquid droplets or solid particles comprising the agents or salts. The liquid droplets or solid particles should have a particle size in the range of about 0.5 to about 5 microns. The solid particles can be obtained by processing the solid compound of any Formula described herein, or a salt thereof, in any appropriate manner known in the art, such as by micronization. The size of the solid particles or droplets will be, for example, from about 1 to about 2 microns. In this respect, commercial nebulizers are available to achieve this purpose.

A pharmaceutical formulation suitable for administration as an aerosol may be in the form of a liquid, the formulation will comprise a water-soluble compound of any Formula described herein, or a salt thereof, in a carrier which comprises water. A surfactant may be present which lowers the surface tension of the formulation sufficiently to result in the formation of droplets within the desired size range when subjected to nebulization.

Peroral compositions also include liquid solutions, emulsions, suspensions, and the like. The pharmaceutically acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol, and water. For a suspension, typical suspending agents include methyl cellulose, sodium carboxymethyl cellulose, tragacanth, and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Pharmaceutical compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, waxes, and shellac.

Other compositions useful for attaining systemic delivery of the subject agents include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

The compositions of this invention can also be administered topically to a subject, e.g., by the direct laying on or spreading of the composition on the epidermal or epithelial tissue of the subject, or transdermally via a “patch”. Such compositions include, for example, lotions, creams, solutions, gels and solids. These topical compositions may comprise an effective amount, usually at least about 0.1%, or evan from about 1% to about 5%, of a compound of the invention. Suitable carriers for topical administration typically remain in place on the skin as a continuous film, and resist being removed by perspiration or immersion in water. Generally, the carrier is organic in nature and capable of having dispersed or dissolved therein the therapeutic compound. The carrier may include pharmaceutically acceptable emolients, emulsifiers, thickening agents, solvents and the like.

Active agents are administered at a therapeutically effective dosage sufficient to inhibit detrimental protein aggregation in a subject. A “therapeutically effective” dosage inhibits detrimental protein aggregation by, for example, at least about 20%, or by at least about 40%, or even by at least about 60%, or by at least about 80% relative to untreated subjects. In the case of, e.g., Parkinson's s patient, a “therapeutically effective” dosage stabilizes cognitive function or prevents a further decrease in cognitive function (i.e., preventing, slowing, or stopping disease progression). The present invention accordingly provides therapeutic drugs. By “therapeutic” or “drug” is meant a compound having a beneficial ameliorative or prophylactic effect on a specific disease or condition in a living human or non-human animal.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50, and usually a larger therapeutic index is more efficacious. While agents that exhibit toxic side effects may be used, care should be taken to design a delivery system that targets such agents to the site of affected tissue in order to minimize potential damage to uninfected cells and, thereby, reduce side effects.

It is understood that appropriate doses depend upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses depend upon the potency with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The ability of a compound to inhibit protein aggregation can be evaluated in an animal model system that may be predictive of efficacy in inhibiting protein aggegation in human diseases, such as a transgenic mouse expressing human α-synuclein or other relevant animal models where protein aggregation is seen, such as those described herein. Likewise, the ability of a compound to prevent or reduce cognitive impairment in a model system may be indicative of efficacy in humans. Alternatively, the ability of a compound can be evaluated by examining the ability of the compound to inhibit protein aggegation formation in vitro, e.g., using a fibrillogenesis assay such as that described herein, including a ThT, CD, or EM assay.

Blood-Brain Barrier

The agents of the invention can be formulated to ensure proper distribution in vivo. For example, the blood-brain barrier (BBB) excludes many highly hydrophilic agents. To ensure that the more hydrophilic therapeutic agents of the invention cross the BBB, they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties”), thus providing targeted drug delivery (see, e.g., V. V. Ranade (1989) J. Clin. Pharmacol. 29:685).

Exemplary targeting moieties include folate or biotin (see, e.g., U.S. Pat. No. 5,416,016 to Low et al.); mannosides (Umezawa et al. (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J Physiol. 1233:134); gp120 (Schreier et aL (1994) J. Biol. Chem. 269:9090); see also K. Keinanen; M. L. Laukkanen (1994) FEBS Lett. 346:123; J. J. Killion; I. J. Fidler (1994) Immunomethods 4:273. In an embodiment, the therapeutic agents of the invention are formulated in liposomes; which may include a targeting moiety.

To ensure that agents of the invention cross the BBB, they may be coupled to a BBB transport vector (for review of BBB transport vectors and mechanisms, see Bickel, et al., Adv. Drug Delivery Reviews, vol. 46, pp. 247-279, 2001). Exemplary transport vectors include cationized albumin or the OX26 monoclonal antibody to the transferrin receptor; these proteins undergo absorptive-mediated and receptor-mediated transcytosis through the BBB, respectively.

Examples of other BBB transport vectors that target receptor-mediated transport systems into the brain include factors such as insulin, insulin-like growth factors (IGF-I, IGF-II), angiotensin II, atrial and brain natriuretic peptide (ANP, BNP), interleukin I (IL-1) and transferrin. Monoclonal antibodies to the receptors which bind these factors may also be used as BBB transport vectors. BBB transport vectors targeting mechanisms for absorptive-mediated transcytosis include cationic moieties such as cationized LDL, albumin or horseradish peroxidase coupled with polylysine, cationized albumin or cationized inimunoglobulins. Small basic oligopeptides such as the dynorphin analogue E-2078 and the ACTH analogue ebiratide can also cross the brain via absorptive-mediated transcytosis and are potential transport vectors.

Other BBB transport vectors target systems for transporting nutrients into the brain. Examples of such BBB transport vectors include hexose moieties, e.g. glucose, monocarboxylic acids, e.g. lactic acid, neutral amino acids, e.g. phenylalanine, amines, e.g. choline, basic amino acids, e.g. arginine, nucleosides, e.g. adenosine, purine bases, e.g. adenine, and thyroid hormone, e.g triiodothyridine. Antibodies to the extracellular domain of nutrient transporters can also be used as transport vectors. Other possible vectors include angiotensin II and ANP; which may be involved in regulating BBB permeability.

In some cases, the bond linking the therapeutic compound to the transport vector may be cleaved following transport into the brain in order to liberate the biologically active compound. Exemplary linkers include disulfide bonds, ester-based linkages, thioether linkages, amide bonds, acid-labile linkages, and Schiff base linkages. Avidin/biotin linkers, in which avidin is covalently coupled to the BBB drug transport vector, may also be used. Avidin itself may be a drug transport vector.

Prodrugs

The present invention is also related to prodrugs of the agents of the Formulae disclosed herein. Prodrugs are agents which are converted in vivo to active forms (see, e.g., R. B. Silverman, 1992, “The Organic Chemistry of Drug Design and Drug Action,” Academic Press, Chp. 8). Prodrugs can be used to alter the biodistribution (e.g., to allow agents which would not typically enter the reactive site of the protease) or the pharmacokinetics for a particular compound. For example, a carboxylic acid group, can be esterified, e.g., with a methyl group or an ethyl group to yield an ester. When the ester is administered to a subject, the ester is cleaved, enzymatically or non-enzymatically, reductively, oxidatively, or hydrolytically, to reveal the anionic group. An anionic group can be esterified with moieties (e.g., acyloxymethyl esters) which are cleaved to reveal an intermediate compound which subsequently decomposes to yield the active compound. The prodrug moieties may be metabolized in vivo by esterases or by other mechanisms to carboxylic acids.

Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the agents, or by separately reacting the purified compound in its free acid form with a suitable derivatizing compound. Carboxylic acids can be converted into esters via treatment with an alcohol in the presence of a catalyst.

Examples of cleavable carboxylic acid prodrug moieties include substituted and unsubstituted, branched or unbranched lower alkyl ester moieties, (e.g., ethyl esters, propyl esters, butyl esters, pentyl esters, cyclopentyl esters, hexyl esters, cyclohexyl esters), lower alkenyl esters, dilower alkyl-amino lower-alkyl esters (e.g., dimethylaminoethyl ester), acylamino lower alkyl esters, acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, dilower alkyl amides, and hydroxy amides.

Pharmaceutically Acceptable Salts

Certain embodiments of the present agents can contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of agents of the present invention. These salts can be prepared in situ during the final isolation and purification of the agents of the invention, or by separately reacting a purified compound of the invention in its free base form with a suitable organic or inorganic acid, and isolating the salt thus formed.

Representative salts include the hydrohalide (including hydrobromide and hydrochloride), sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, 2-hydroxyethylsulfonate, and laurylsulphonate salts and the like. (See, e.g., Berge et al. (1977) “Pharmaceutical Salts”, J. Pharm. Sci. 66, 1-19).

In other cases, the agents of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of agents of the present invention.

These salts can likewise be prepared in situ during the final isolation and purification of the agents, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines usefuil for the formation of base addition salts include ethylamine, diethylamine, ethylenediamine, ethanolamine,:diethanolamine, piperazine and the like.

### EXAMPLES

The invention is further illustrated by the following examples, which should not be construed as further limiting.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. The contents of all references, issued patents, and published patent applications cited throughout this application are hereby incorporated by reference.

### Example 1

### Assays used to Detect a Detrimental Protein Aggregate Associated with a Protein Aggregation Disorder

Assays for Detecting a Detrimental Protein Aggregate Associated with the Detrimental Accumulation of NAC

Preparation of NAC Peptide

As discussed herein, the acronym NAC refers to a non-amyloid component of the amyloid placques found inADpatients, specifically, a 35 amino acid peptide corresponding to residues 61 to 95 of the α-synuclein protein. NAC peptide was synthesized by Fmoc tertiary butyl chemistry on a Protein Technologies, Inc. peptide synthesizer with a purity of >98%. The peptide content was determined by amino analysis and found to be 71.6%.

Preparations of NAC may contain aggregated material. To eliminate these aggregates and monomerize the peptide, this disaggregation/filtration procedure was adapted from the work of Walsh and Colleagues (J Biol Chem. 1997 Aug. 29; 272 (35):22364-72). The method consists of sonicating the peptide in 1,1,1,3,3,3 Hexafluoroisopropanol (HFIP) to dissolve any structures that may be present. Subsequently, any large aggregates that may remain in the solution are removed by filtration through a 20 nm filter, and stored at −80° C. for later use.

Compound Preparation

2 mM Solutions were prepared in TBSA buffer (Tris 0.02 M, NaCl 0.15 M, NaN₃ 0.005%, pH 7.4) and stored at 4° C. For the assays, compounds were diluted to the desired concentrations in TBSAE buffer (Tris 0.02 M, NaCl 0.15 M, NaN₃ 0.005%, EDTA 100 μM, pH 7.4) and combined with the NAC peptides.

Fibrillogenesis Assays

Fibrillogenesis assays were employed to identify test compounds that inhibit NAC's ability to assemble into fibers. Following removal of HFIP by evaporation under N₂ gas, 20 μM NAC peptide is incubated in the assay buffer (0.02M Tris, 0.15 M NaCl, 0.005% NaN₃, 100 uM EDTA) at 37° C. in a Perkin-Elmer HTS 7000 plus microplate reader with shaking for 1 minute every twenty minutes. The presence of fibers can be monitored by three different methods.

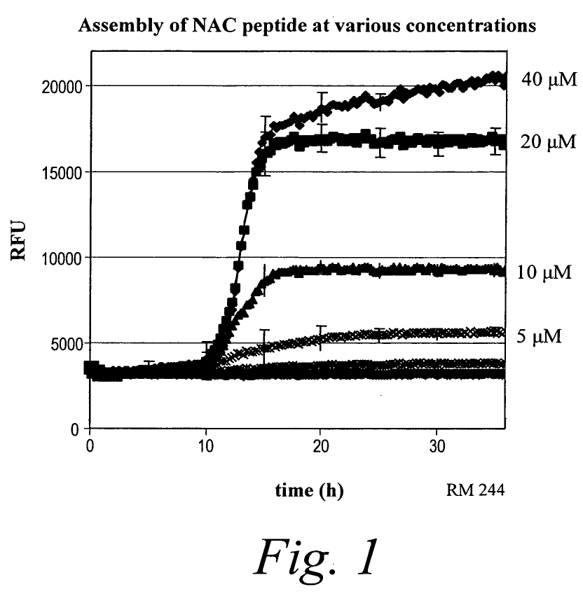

The following assays were set up in clear polystyrene 96 well microplates by combining 125 μL of 40 μM NAC peptide with 125 μL of 200 μM test compound in 0.02M Tris, 0.15 M NaCl, 0.005% NaN₃, 100 μM pH 7.4. The microplates were sealed with a plastic sheet and incubated at 37° C. in a Perkin-Elmer HTS 7000 microplate reader with shaking for 1 minute, every 20 minutes.
Thioflavin T Analysis. To perform this assay 5 μM Thioflavin T is added to the NAC assembly conditions described above. The fluorescence is measured at 430 excitation/485 emission every twenty minutes with a Perkin-Elmer HTS microplate reader for the duration of the assay. The presence of fibers can be detected by an increase in fluorescence as seen in FIG. 1. This assay allows for the identification of compounds that can inhibit this increase in fluorescence upon incubation.
Turbidity Analysis. Alternatively, NAC peptide that has been incubated with test compounds in the appropriate assembly conditions can be analyzed by reading the absorbance at 405 nm to follow the turbidity of the solution which is also an indication of the formation of aggregates.
Circular Dichroism (CD) Analysis. Analysis of NAC peptide during the process of assembly demonstrates that NAC in the absence of test compounds converts from a random coil conformation to a β-sheet/turn conformation after approximately 15-20 hours of incubation as detected by circular dichroism spectroscopy. This assay allows for the identification of compounds that can prevent NAC from adopting a β-sheet/β-turn conformation upon incubation.
At the appropriate time point, during the incubation, the test solutions were transferred to 0.1-cm path length quartz cuvettes and scanned by CD spectroscopy between 190 and 260 nm, with a resolution of 0.1 nm and a bandwidth of 1 nm using a Jasco J-715 spectropolarimeter at 37° C. NAC peptide alone was found to adopt a β-sheet/turn conformation after overnight incubation. Some of the samples with test compounds were observed to have maintained the NAC in a random coil, some were undergoing conformational transition, but had not yet shifted to the β-sheet/β-turn conformation.
Electron Microscopy (EM) Analysis. At the time point at which CD scans are performed a 3 μl aliquot is removed from the sample, spotted onto a Formvar grid and then negatively stained with 4% uranyl acetate. The grids were examined for the presence of fibrils and amorphous aggregates using a JOEL 2000 transmission electron microscope at a 25,000 × magnification. This assay can be used to confirm the absence of fibers in the presence of compounds which inhibit NAC assembly by ThT and CD. The final results were obtained by averaging 10 complete sectors.

### Example 2

### Use of the Thioflavin T Assay to Determine that Isolated NAC Forms β-Sheets

Experiments performed show that Thioflavin T (ThT) could be used for both high throughput and confirmatory work using the NAC peptide. Fluorescent signal indicative of β-sheet formation appeared starting at 10 hours of incubation (FIG. 1). The intensity of the fluorescent signal was directly related to the concentration of the NAC in the solutions and became maximum at 30 μM NAC. A similar ThT profile with a T_{1/2} (time required to obtain a signal equal to half the maximal signal obtained) of ˜15 hours was observed (in 96 well plate format).

### Example 3

### Circular Dichroism and Electron Microscopy of NAC Peptide Conformation

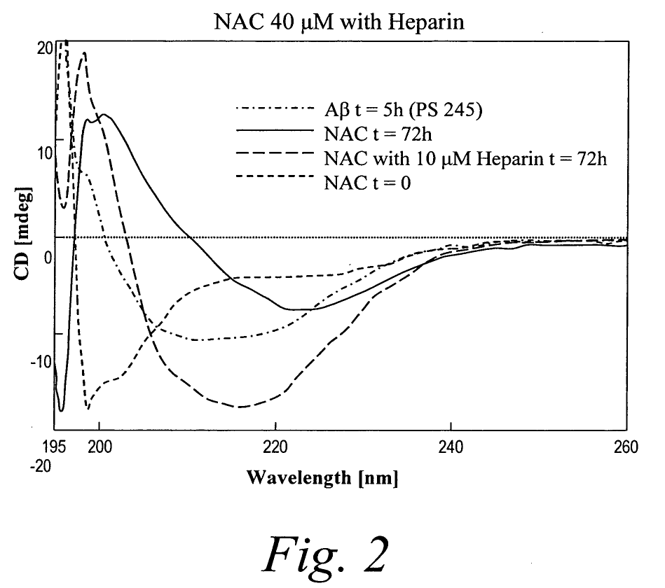

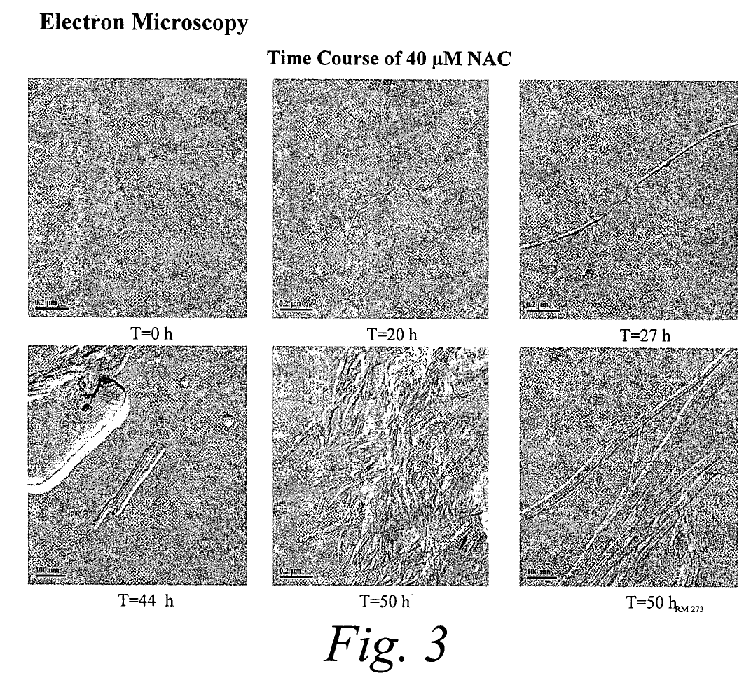

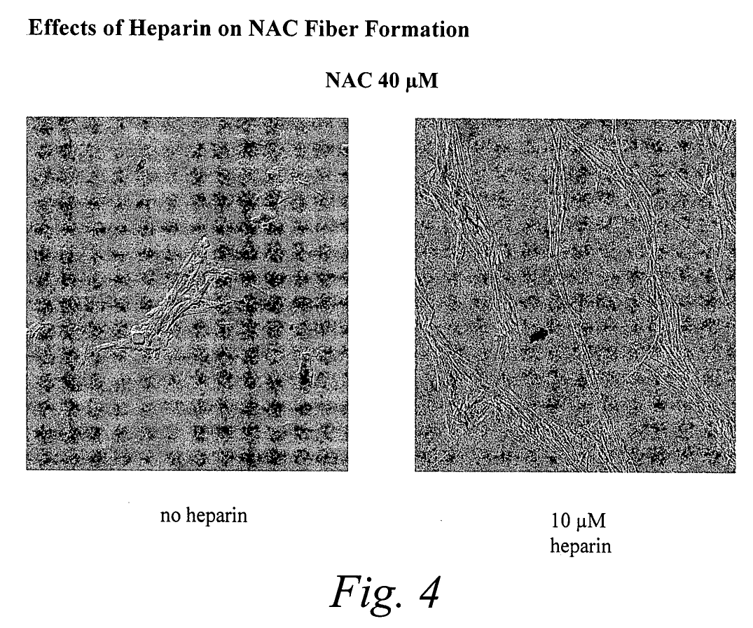

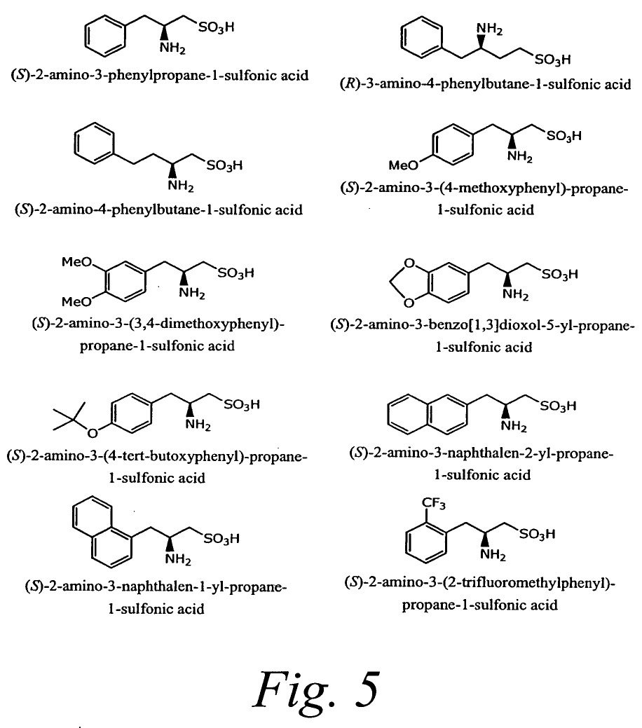

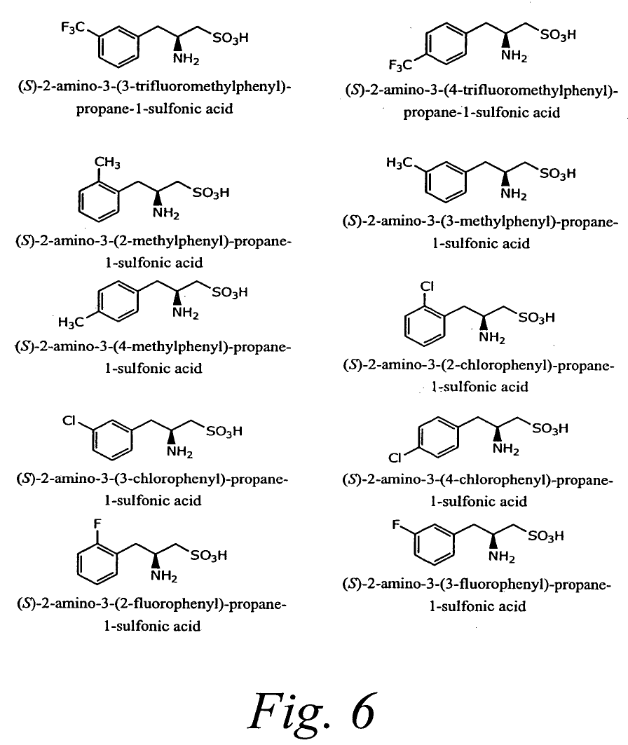

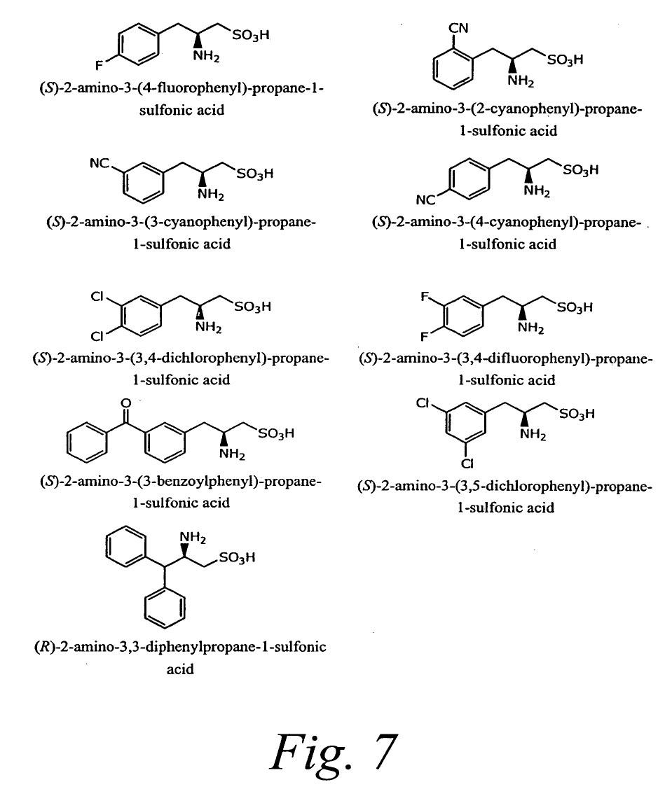

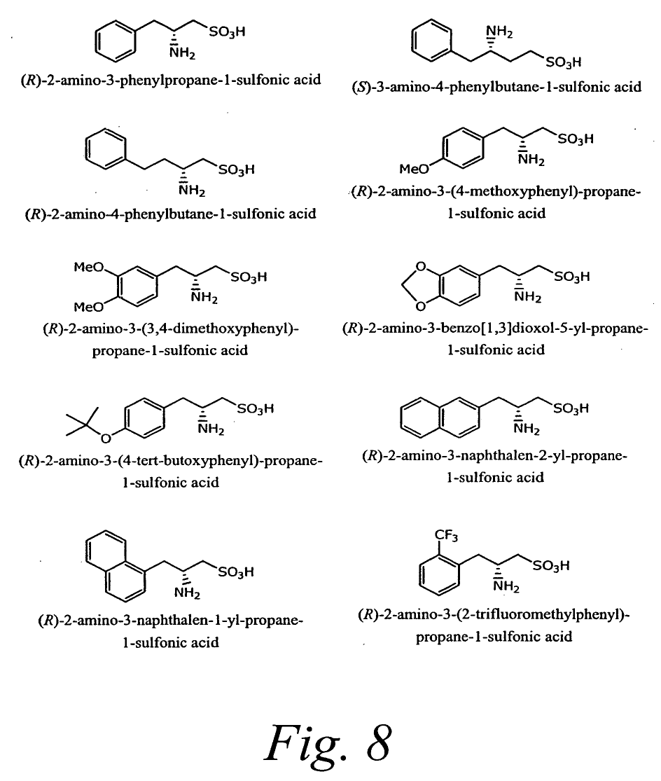

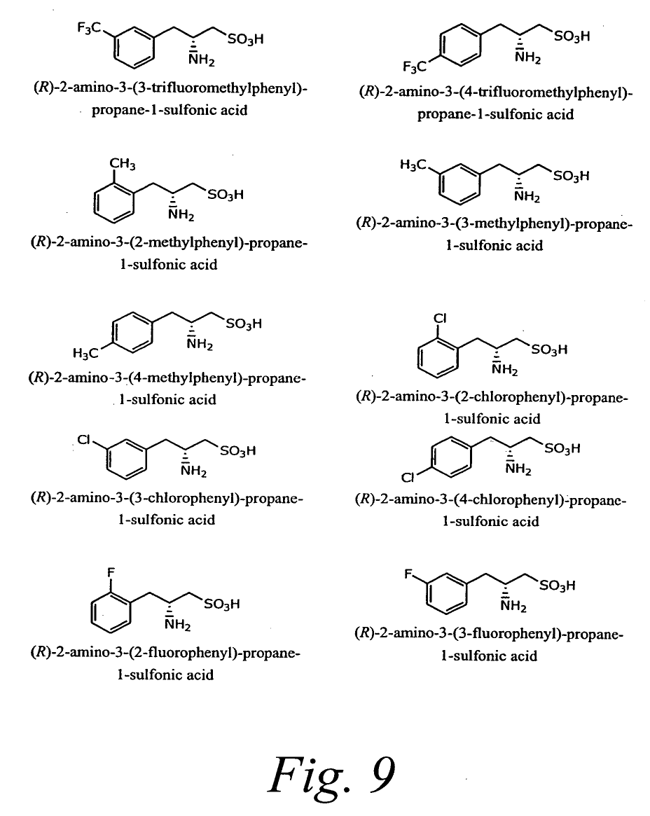

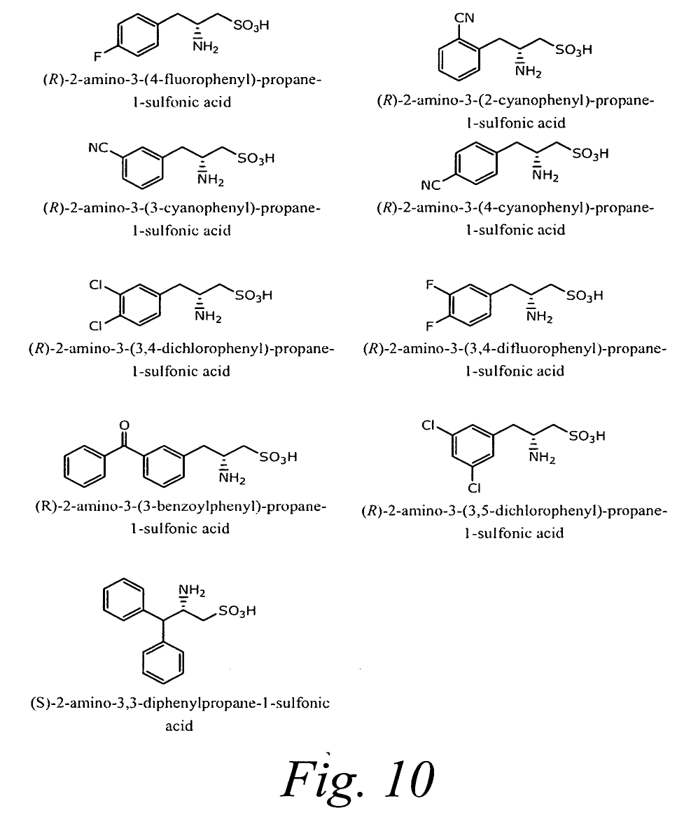

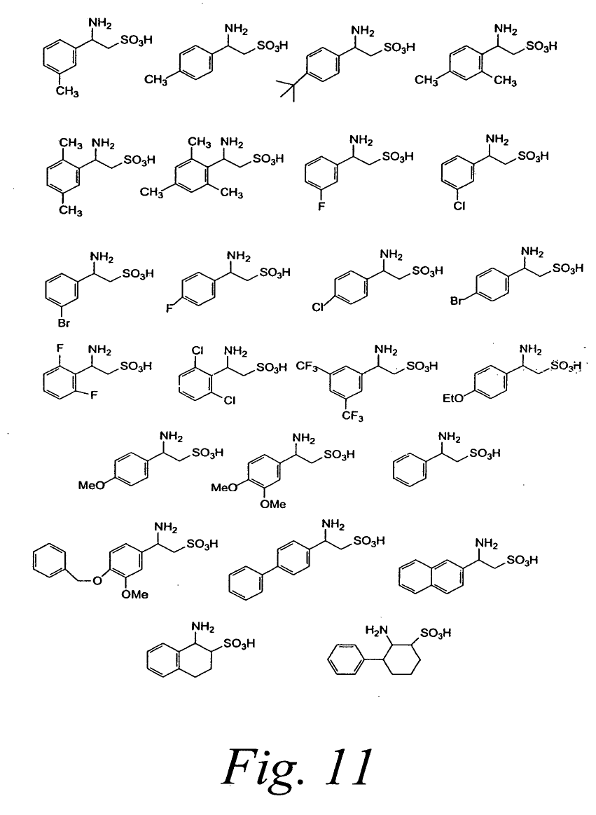

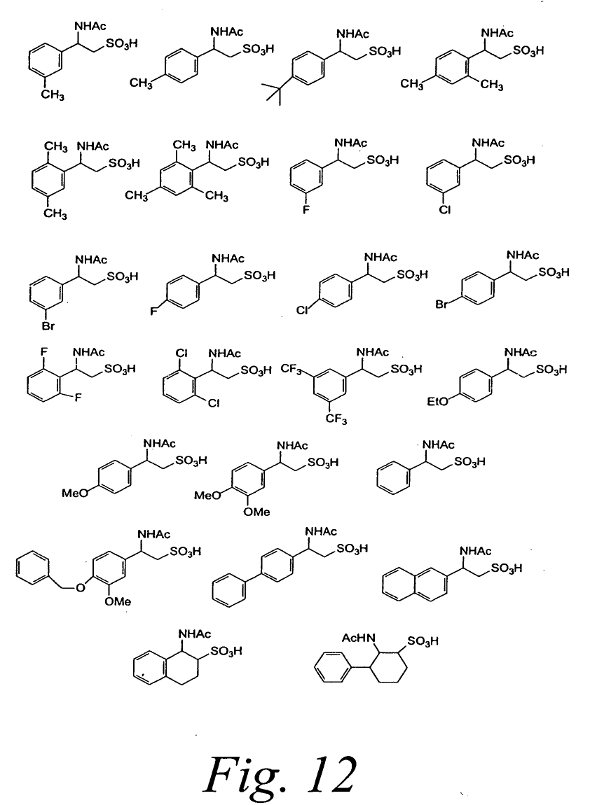

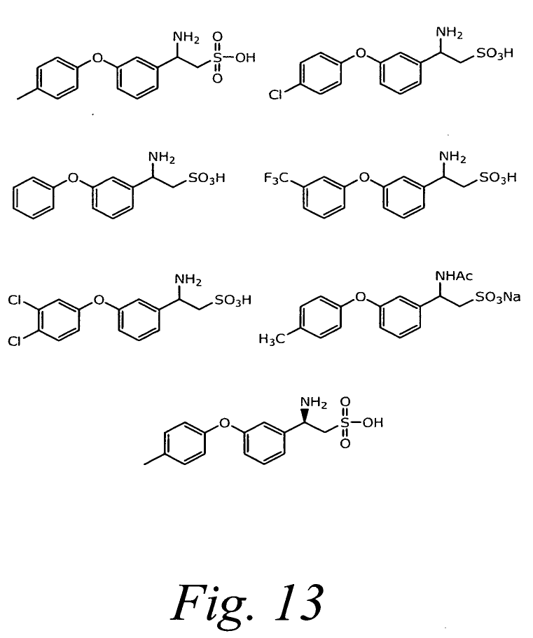

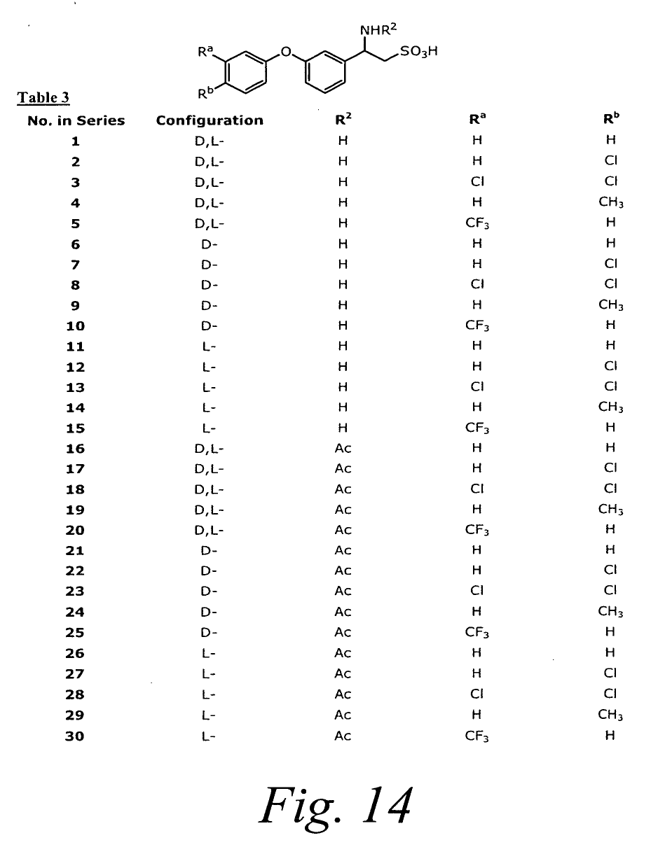

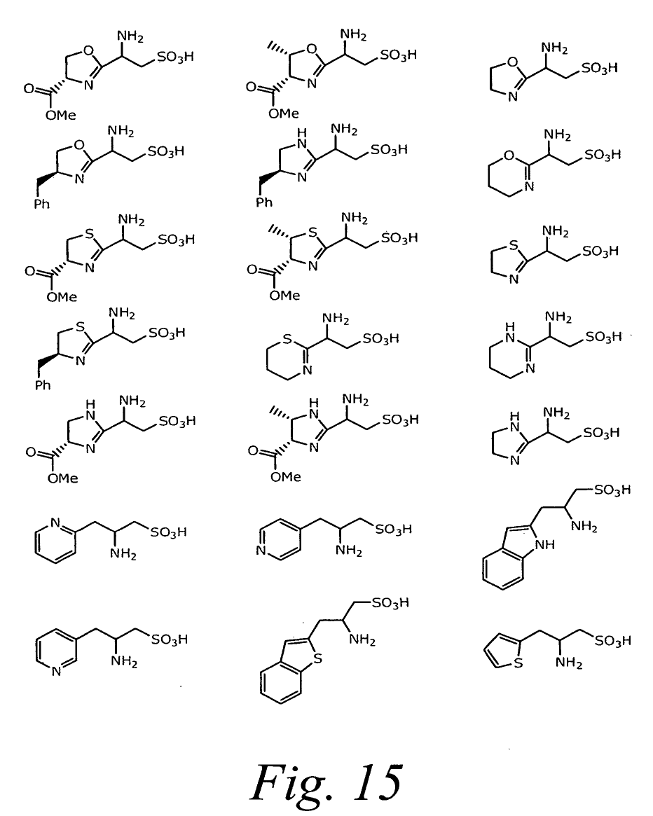

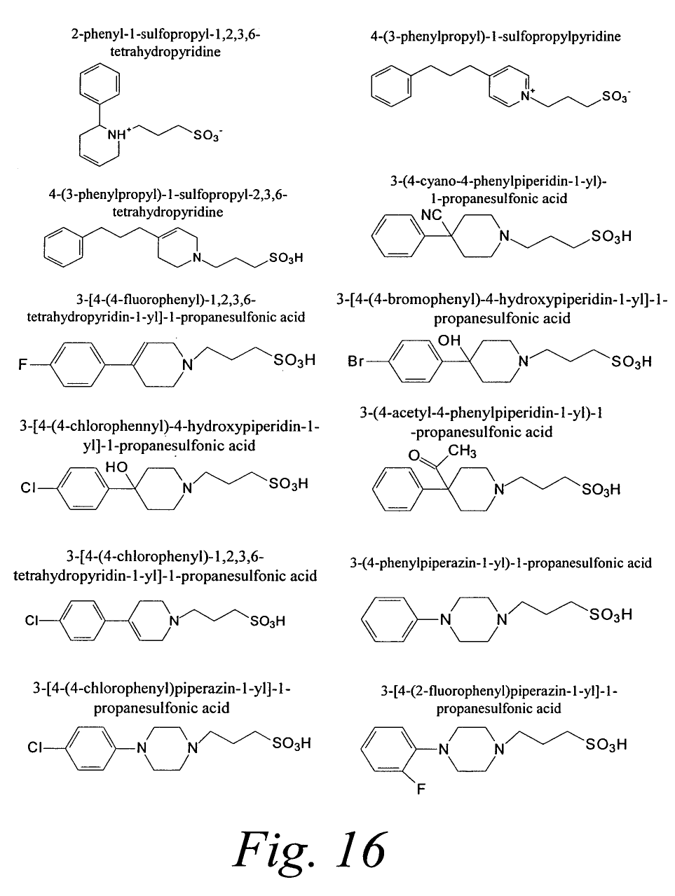

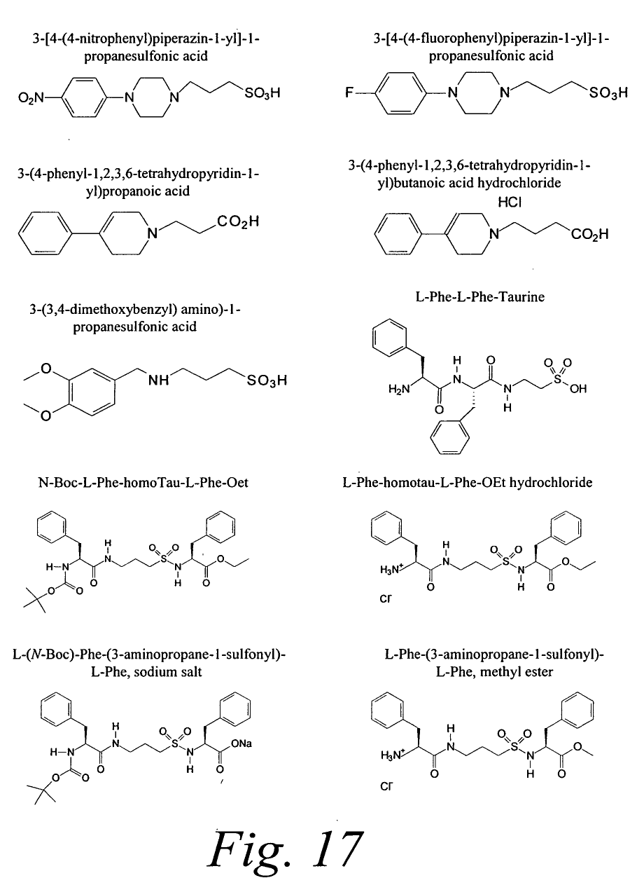

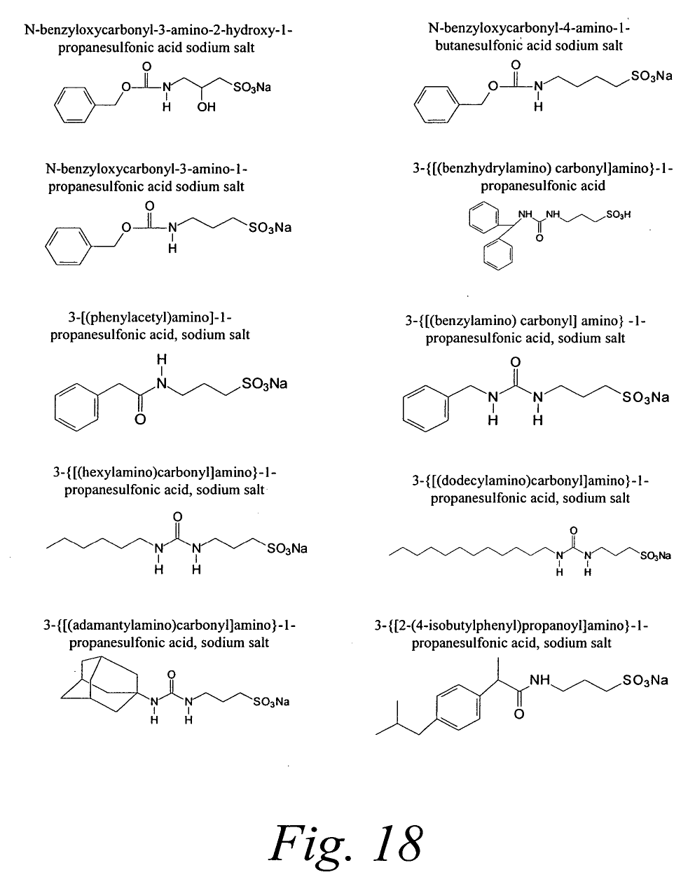

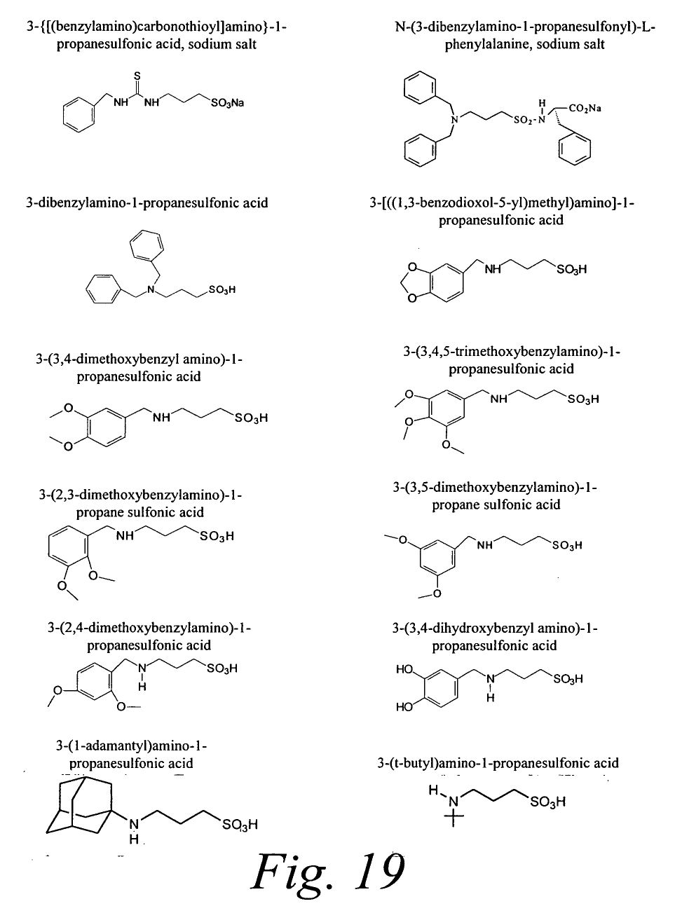

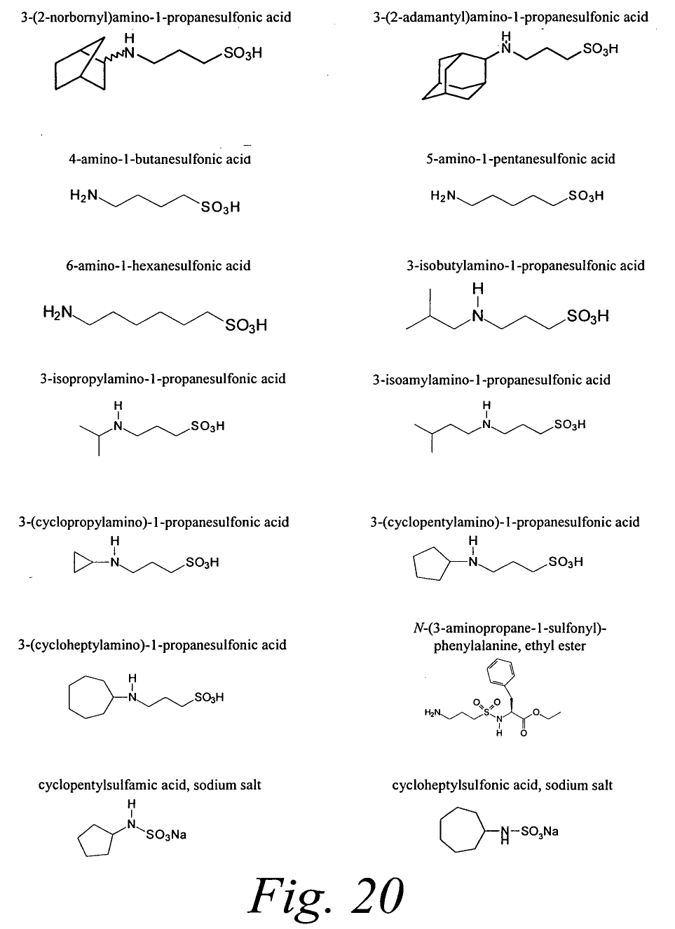

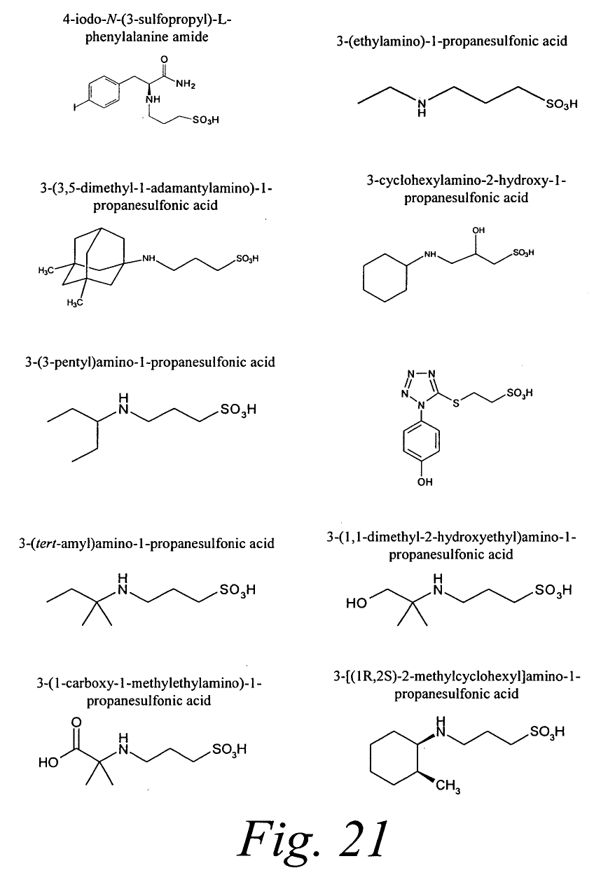

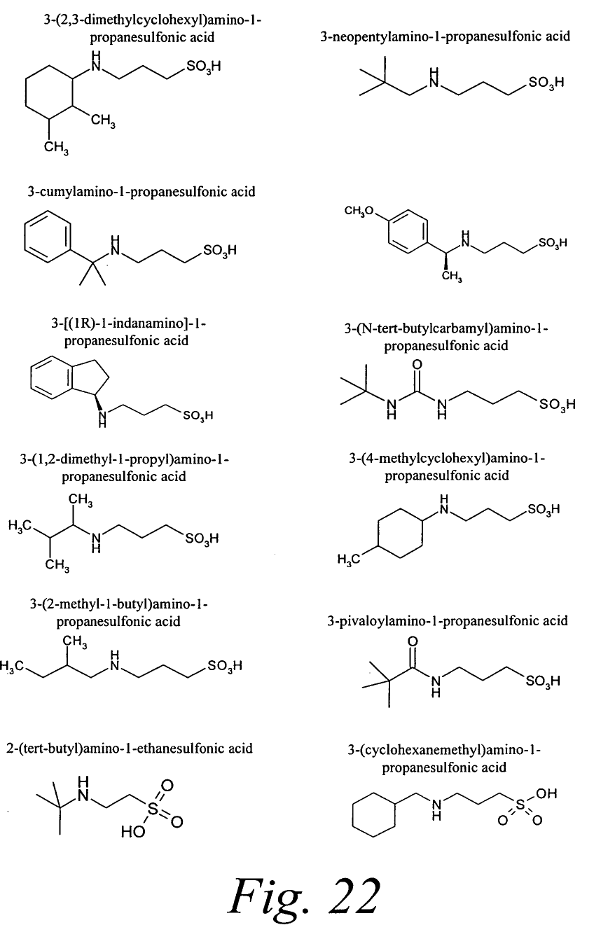

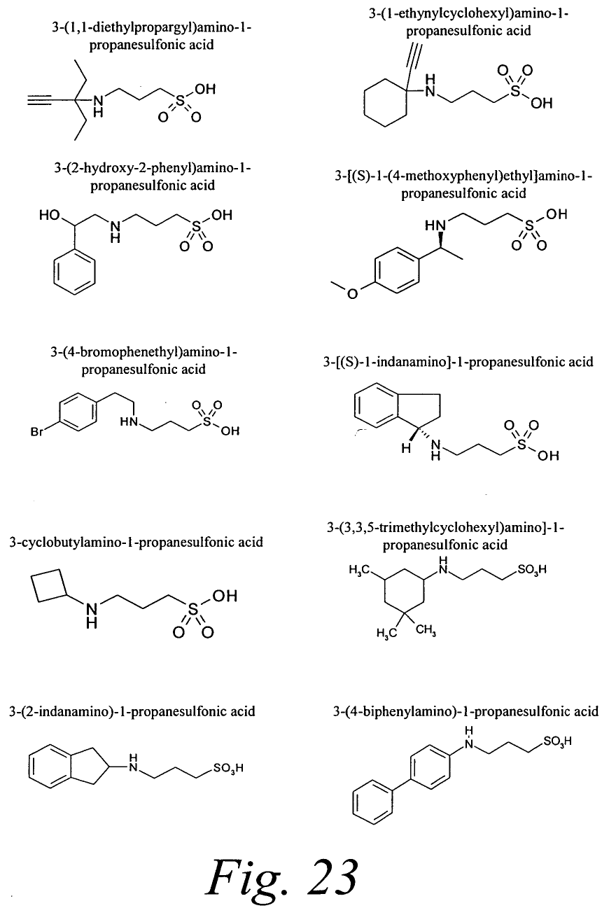

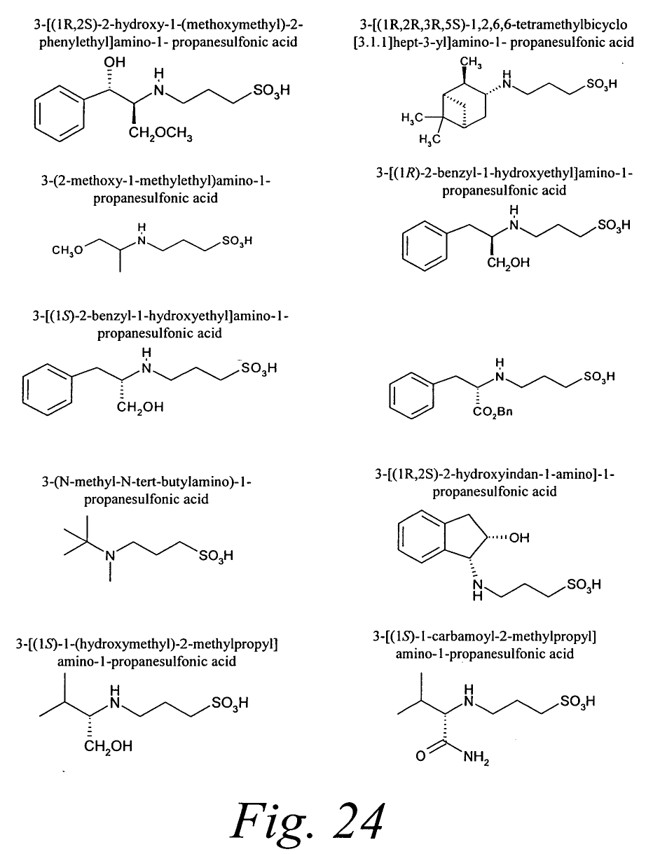

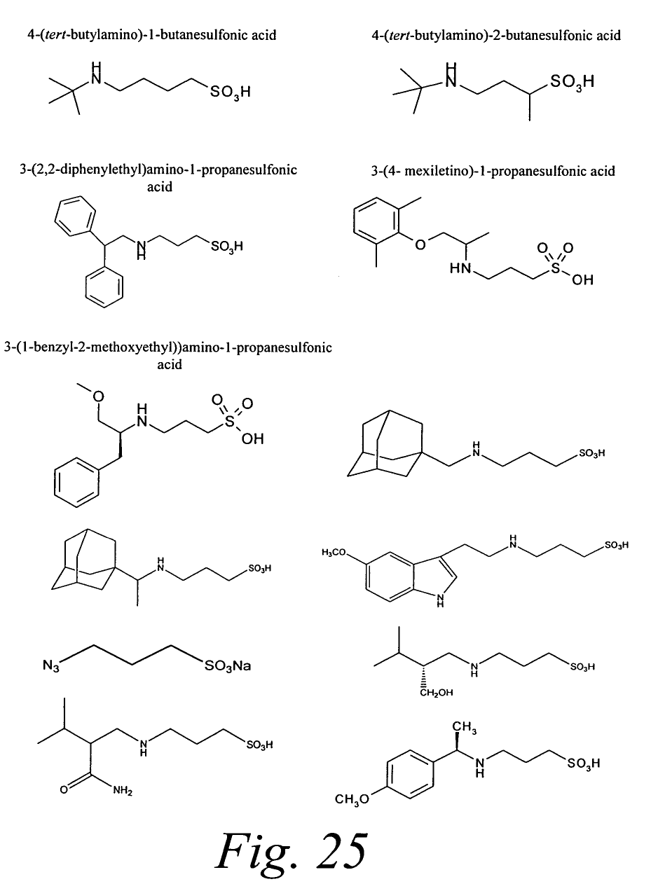

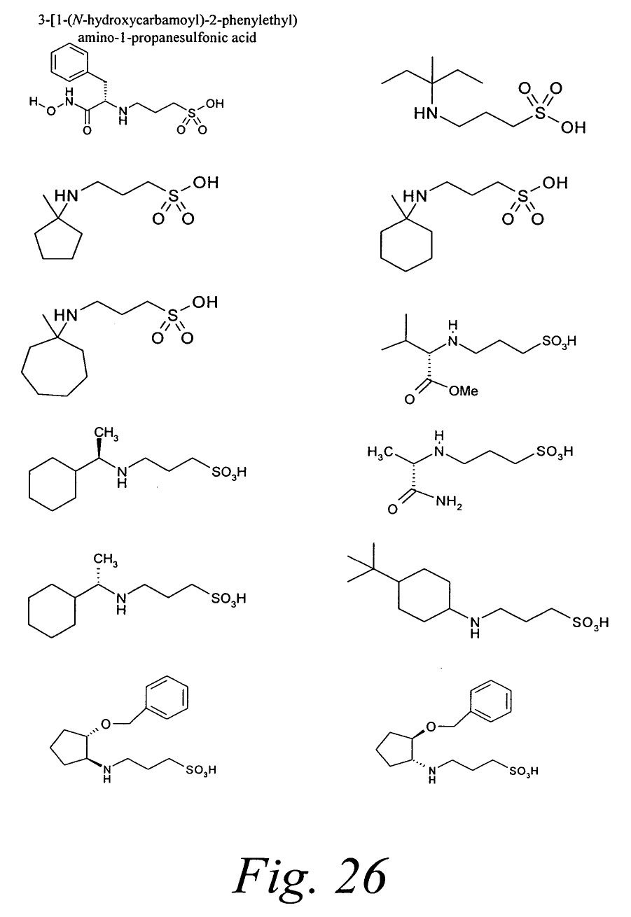

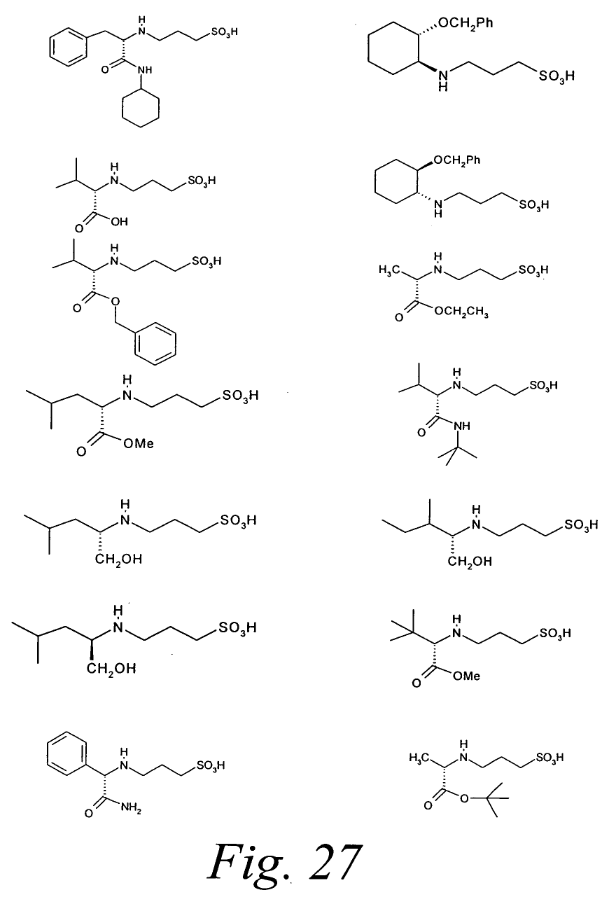

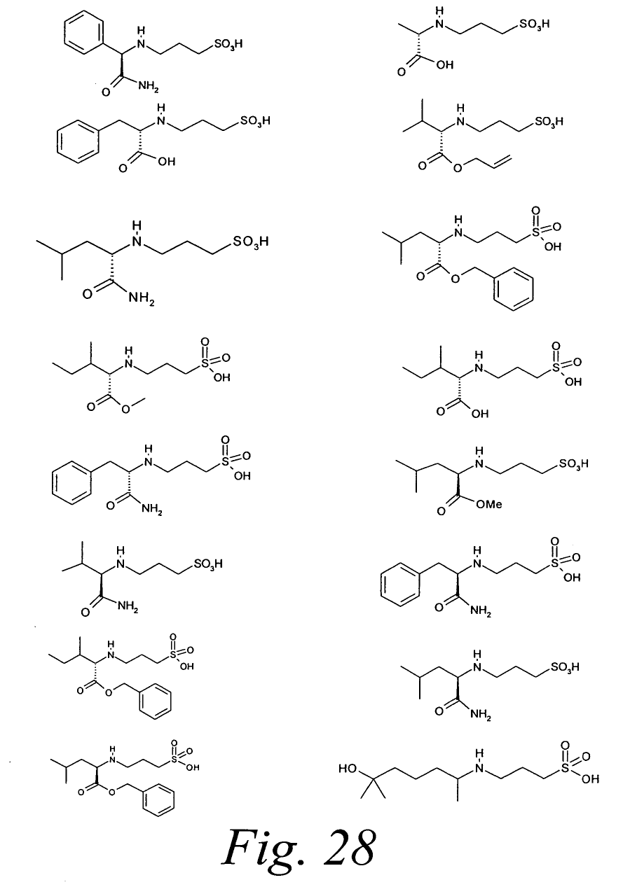

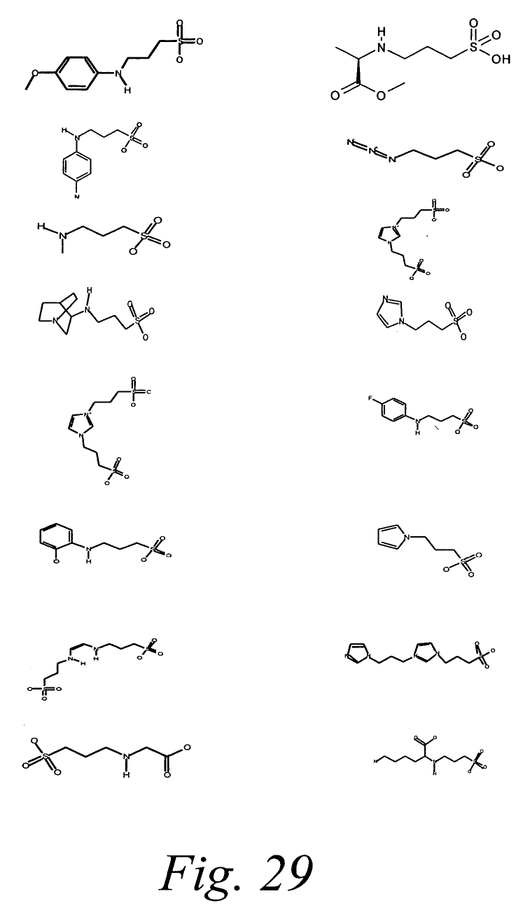

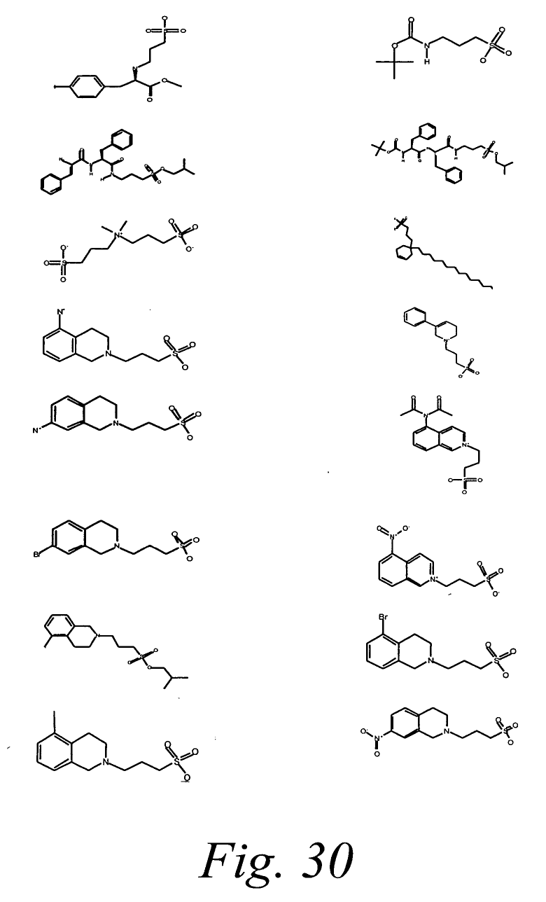

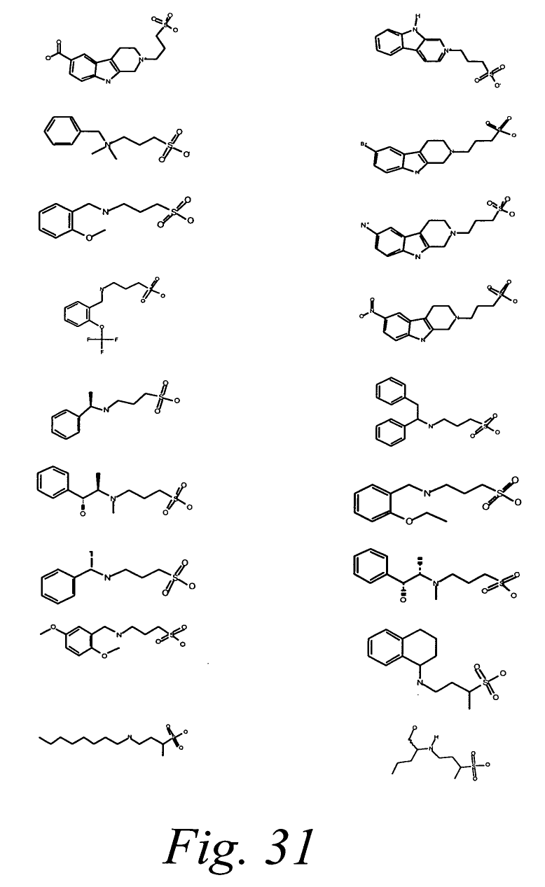

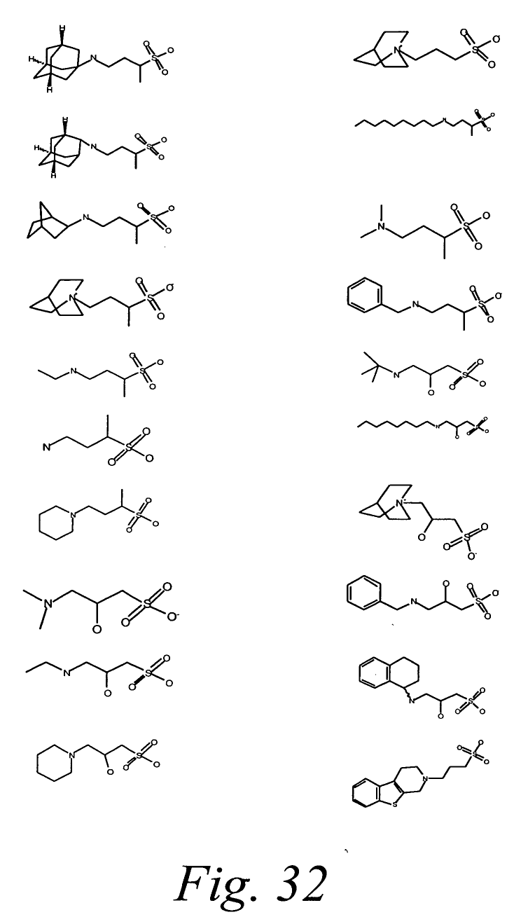

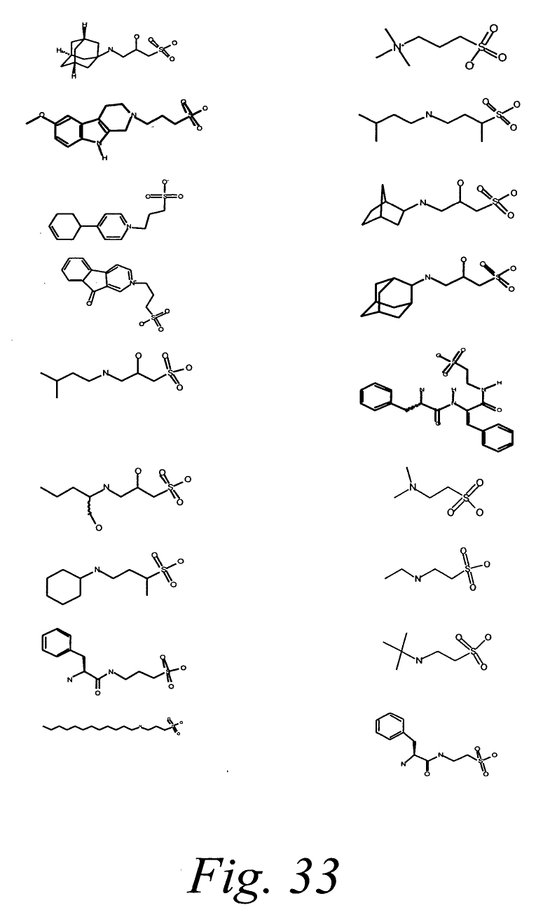

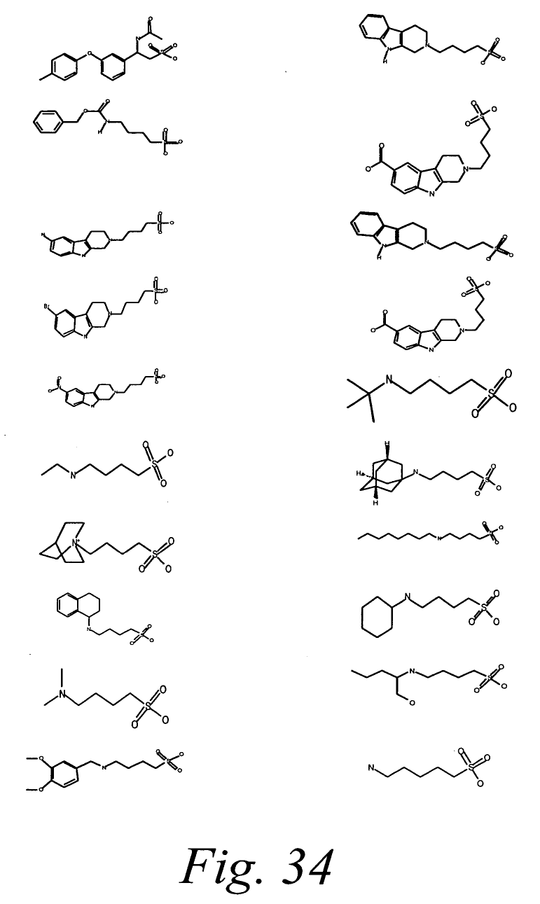

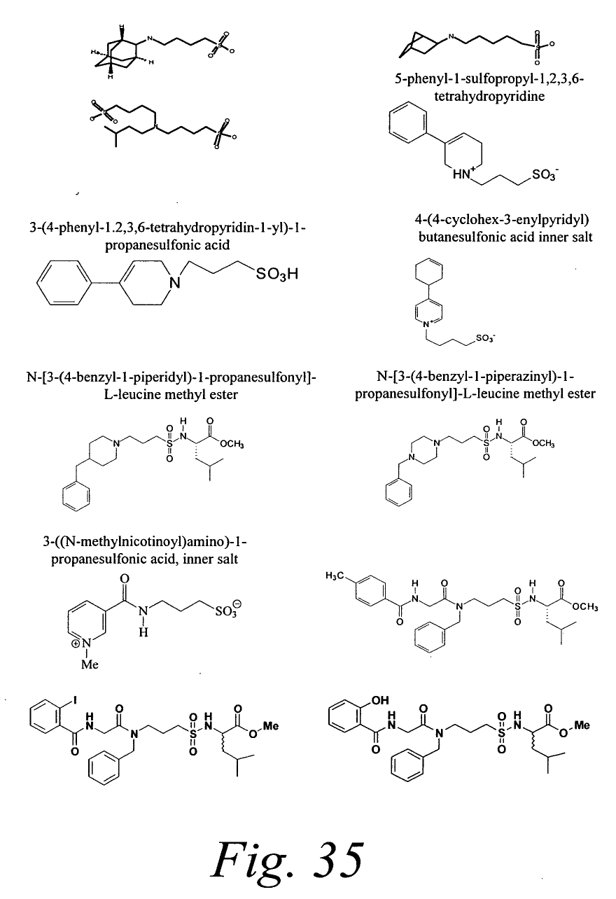

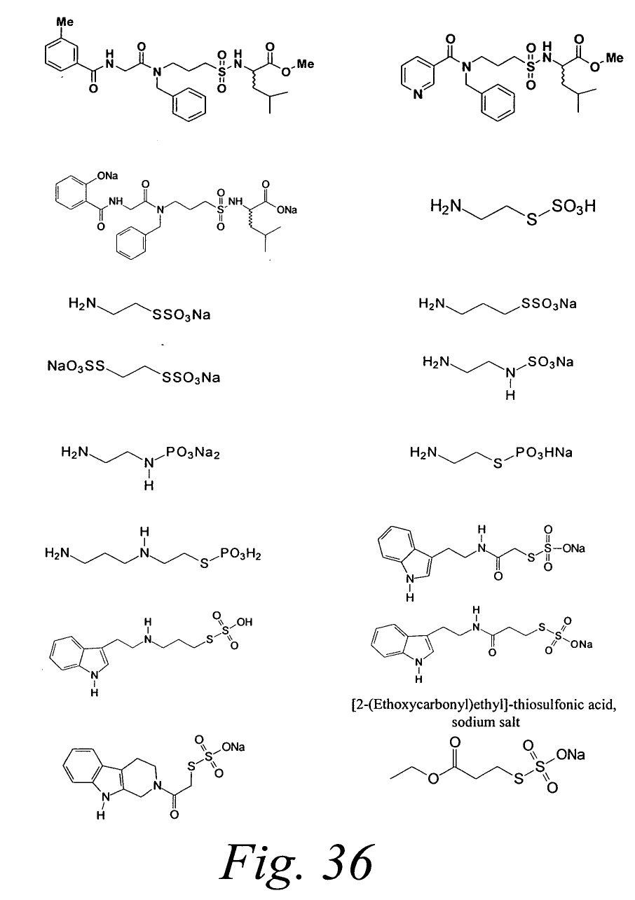

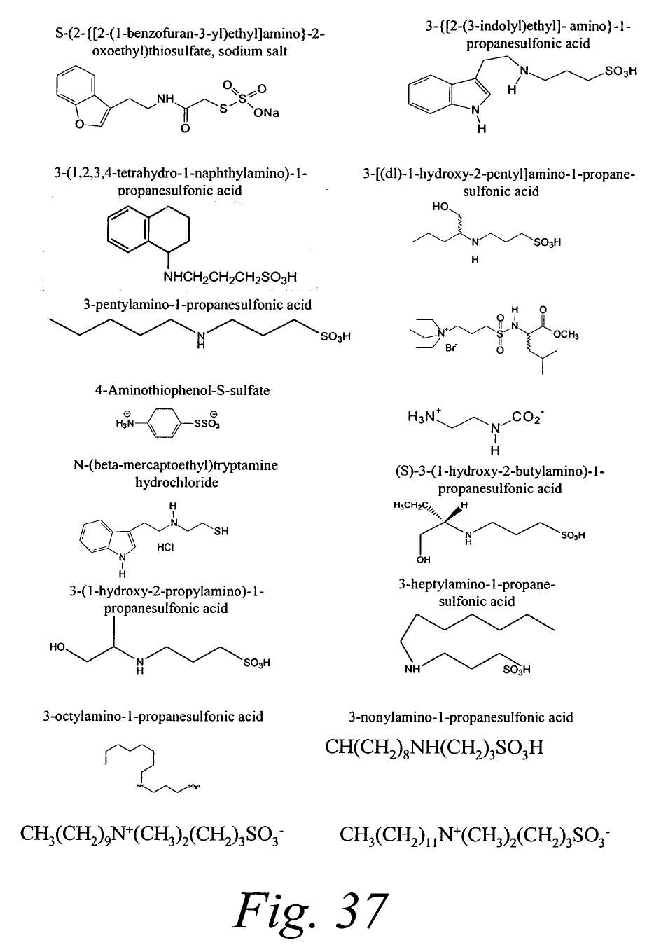

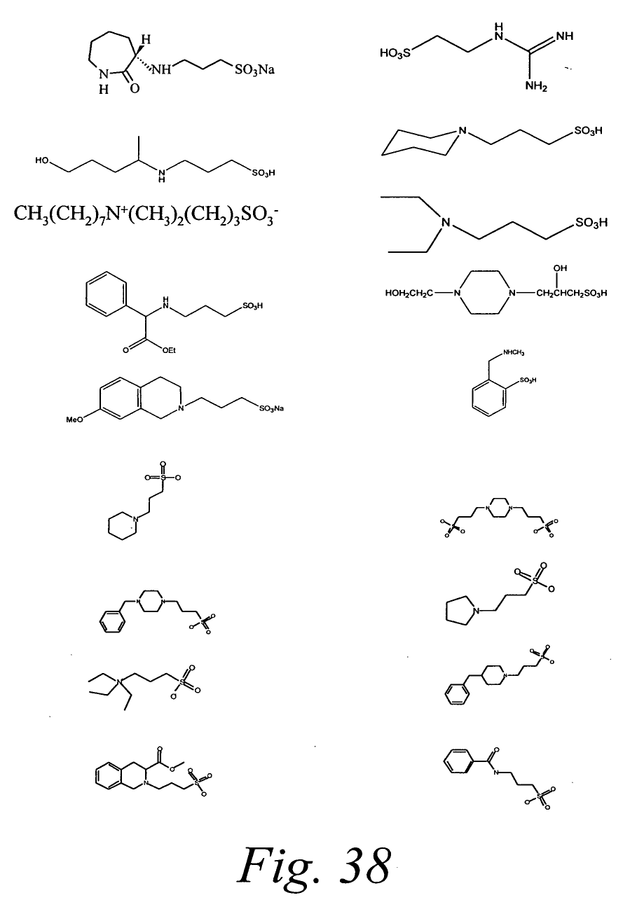

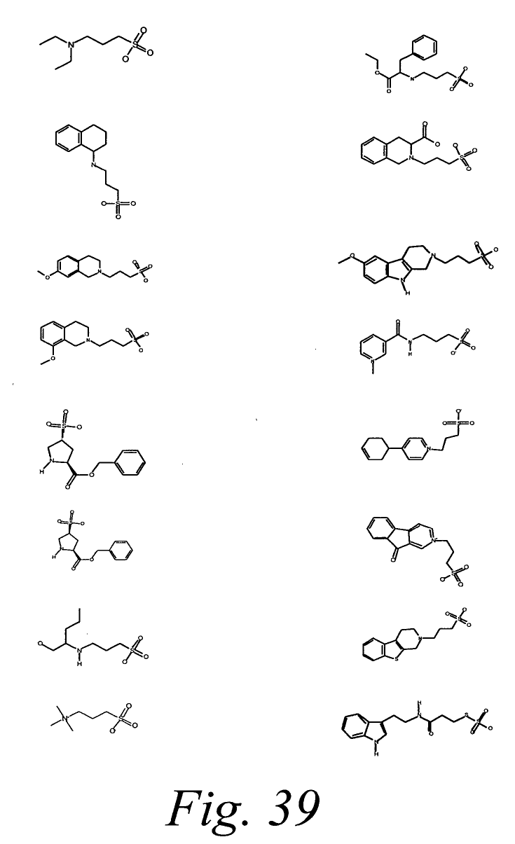

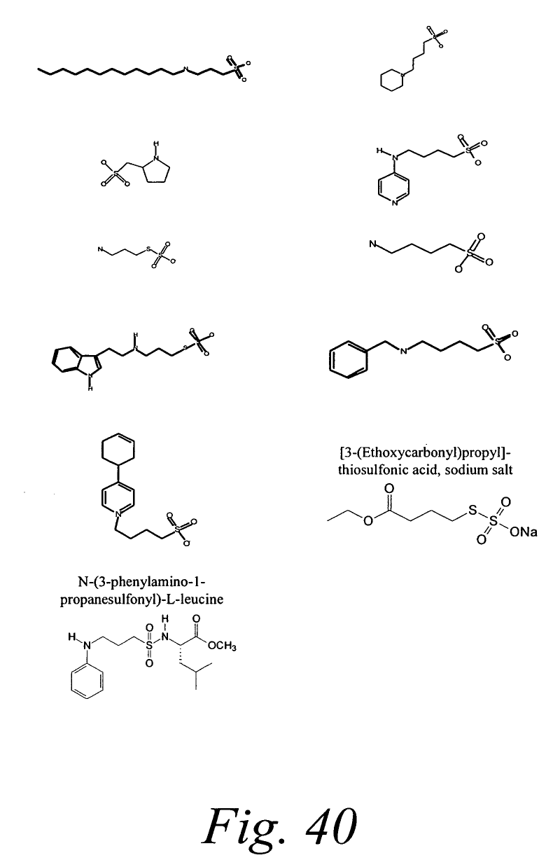

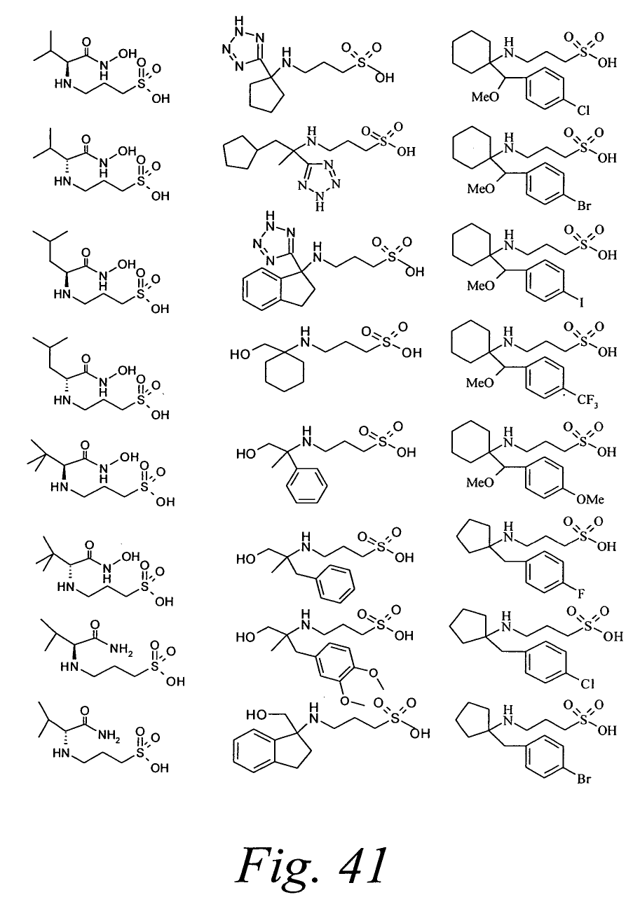

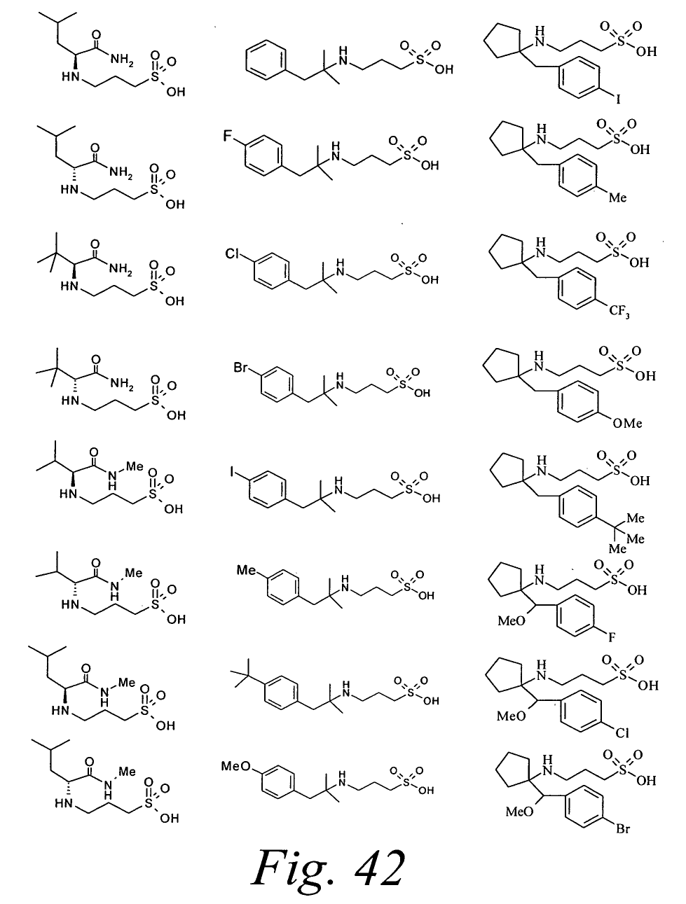

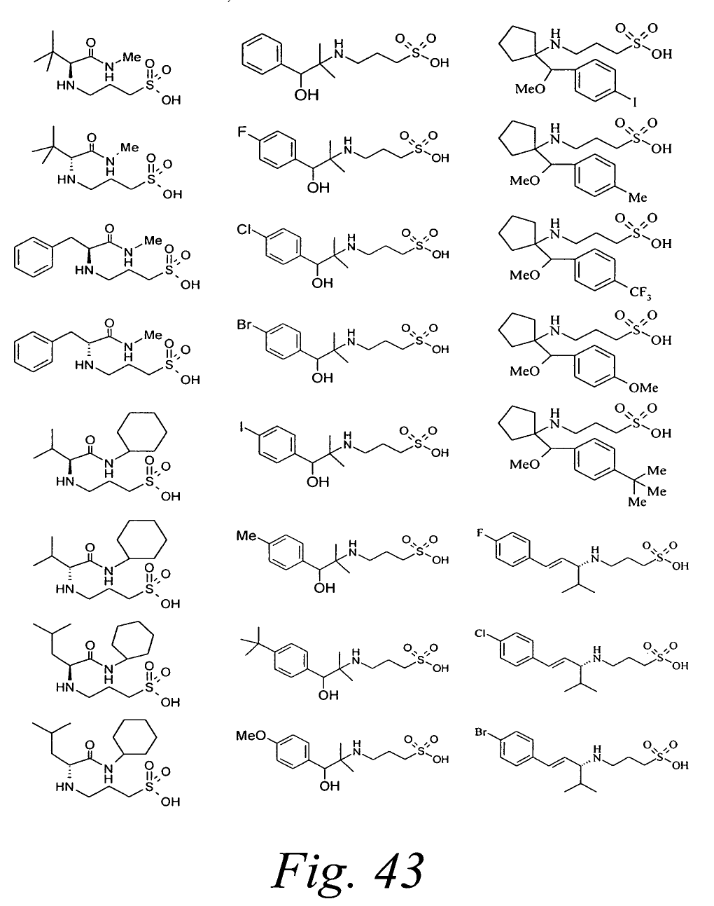

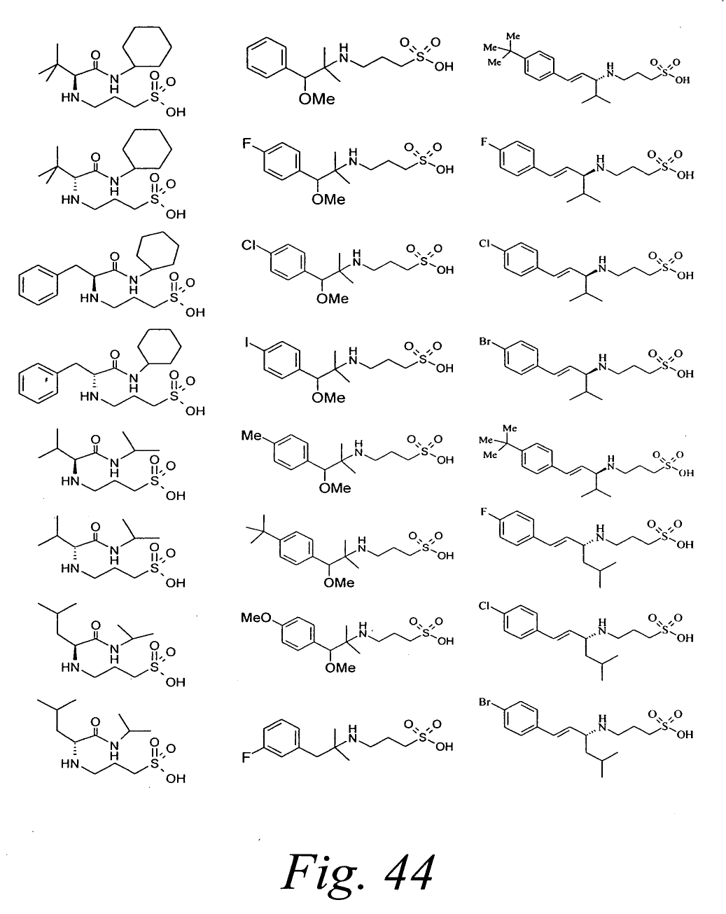

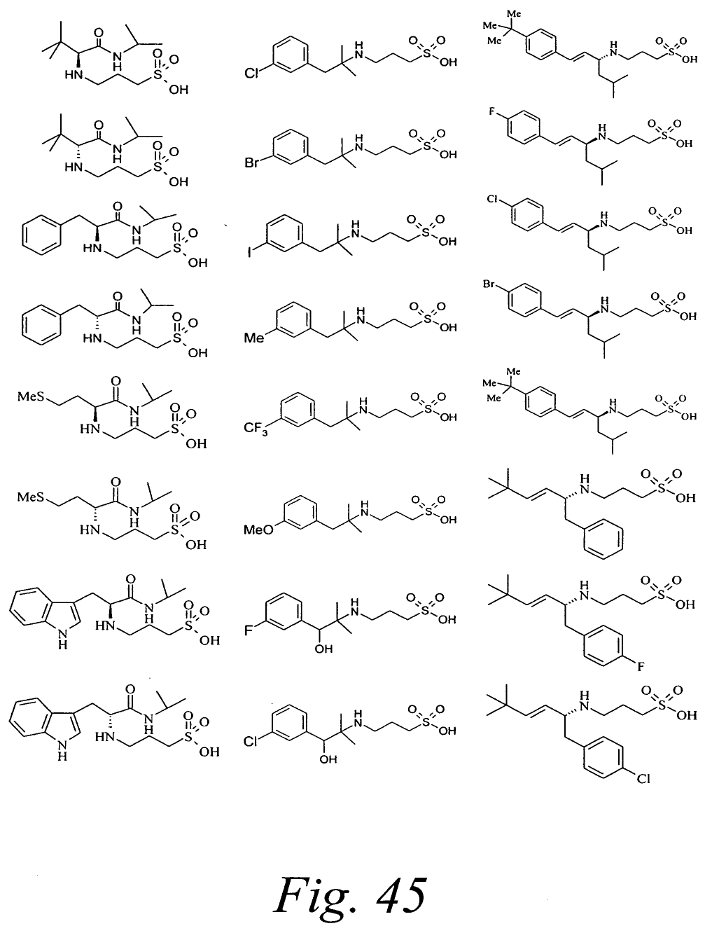

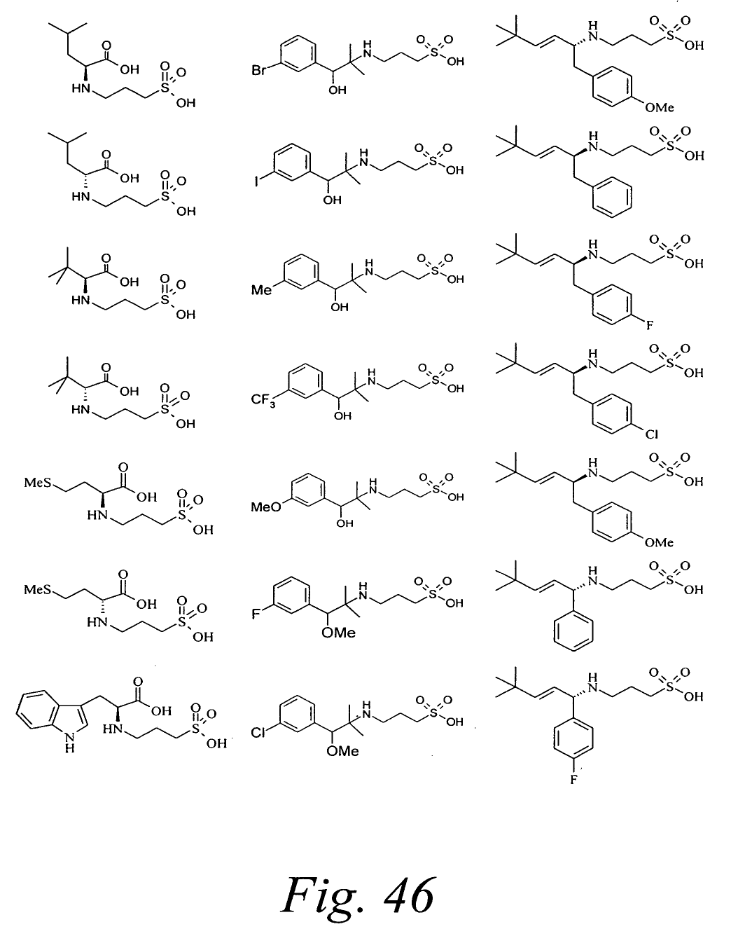

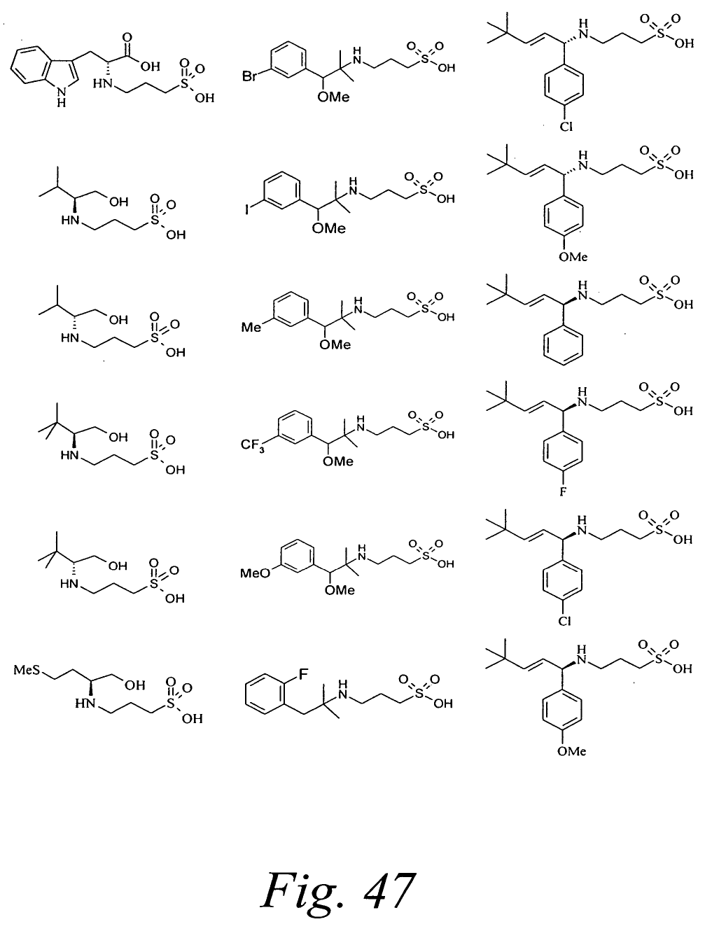

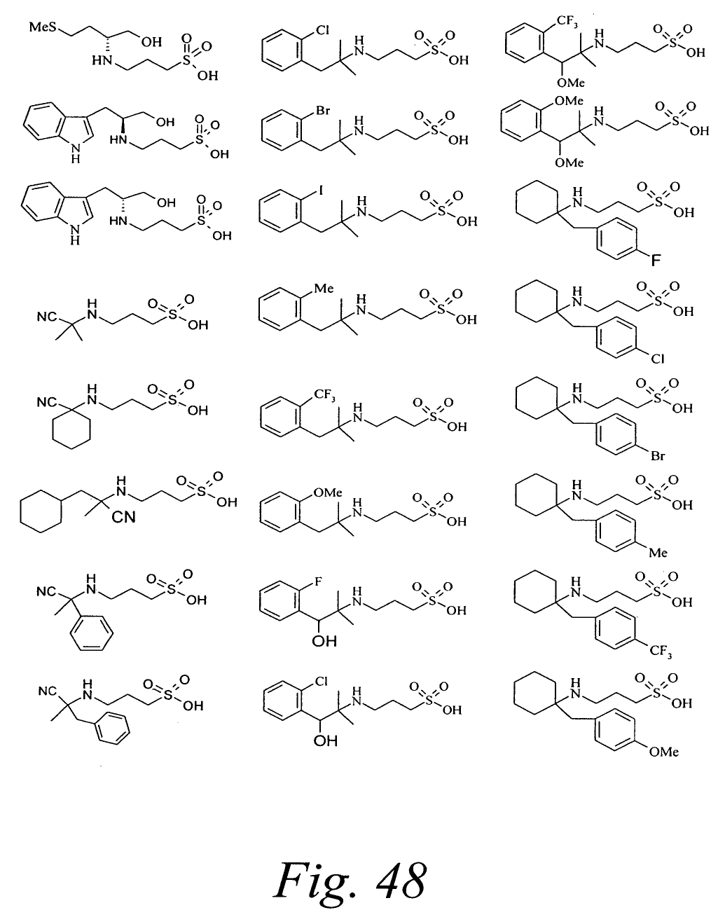

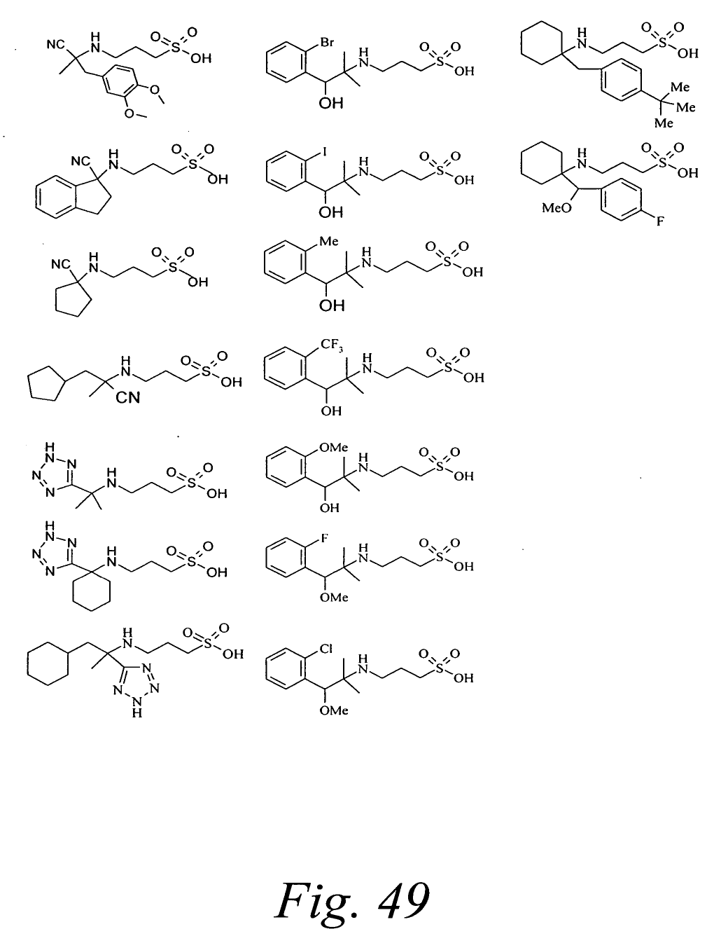

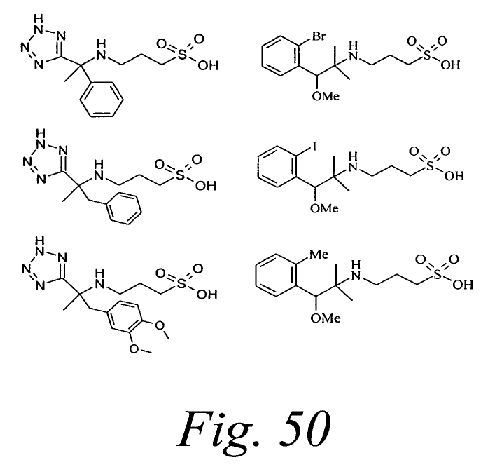

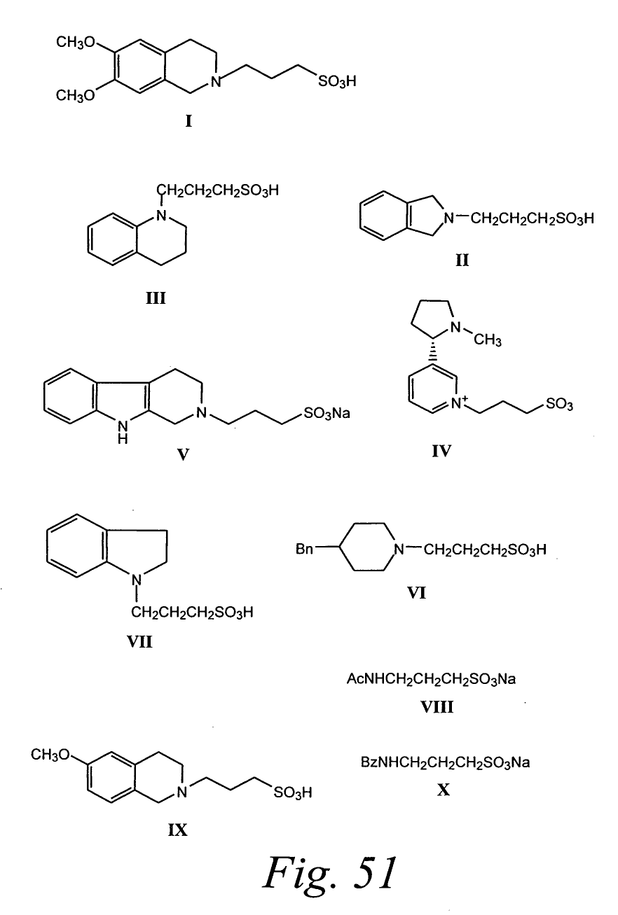

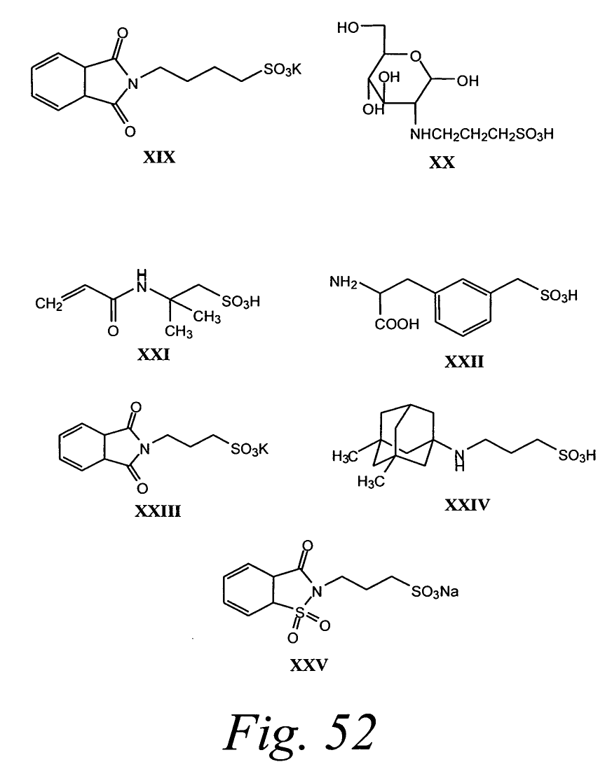

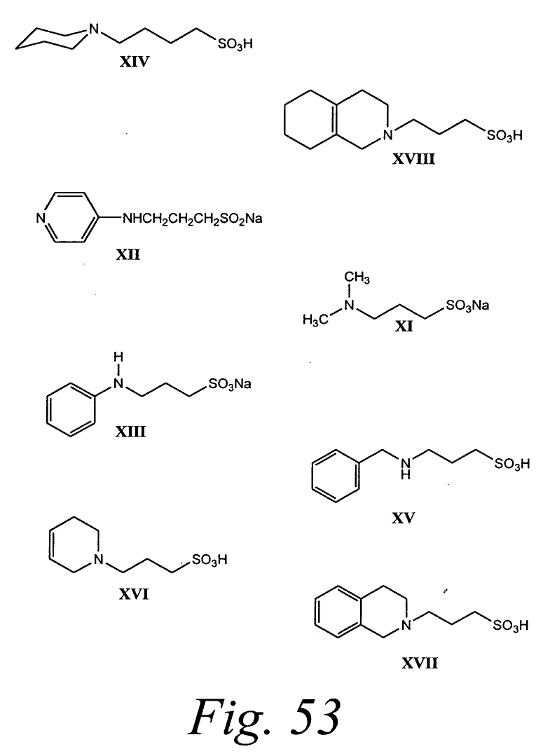

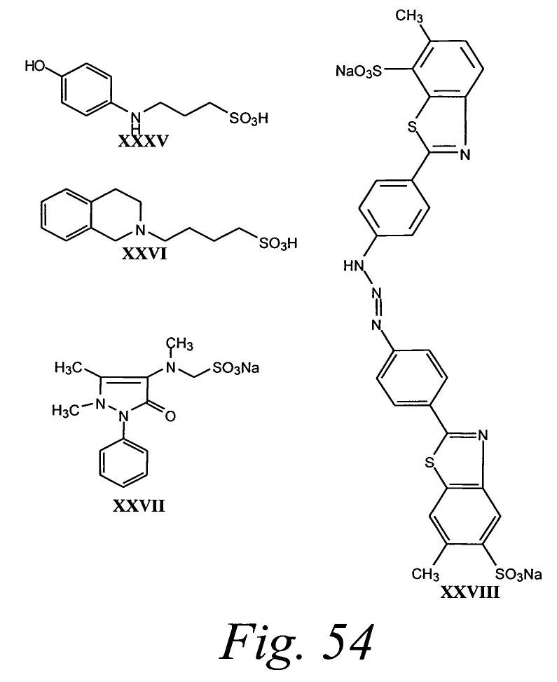

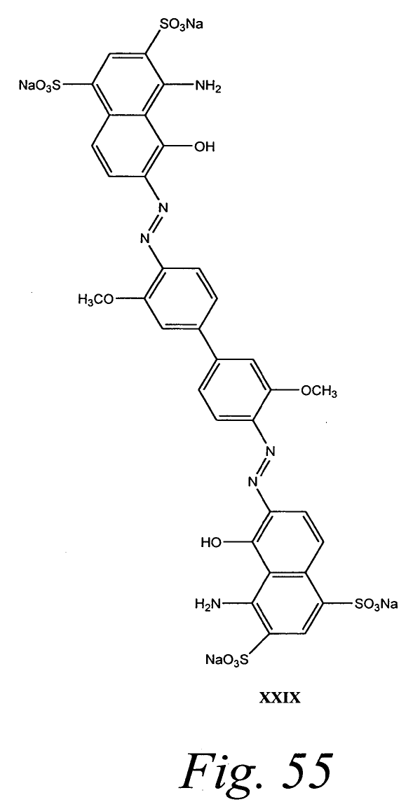

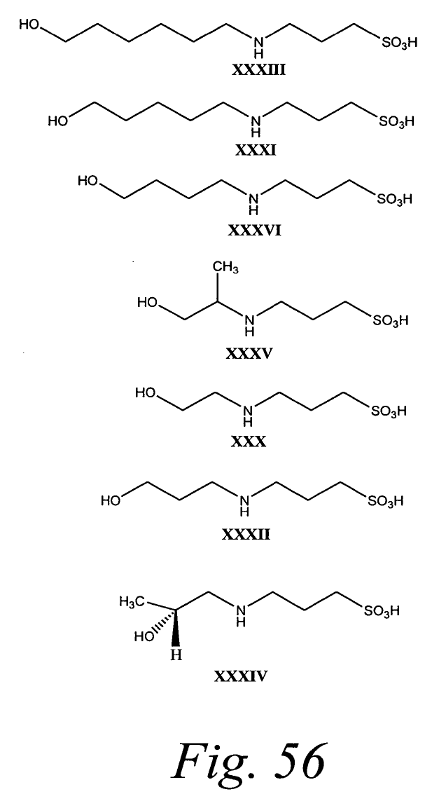

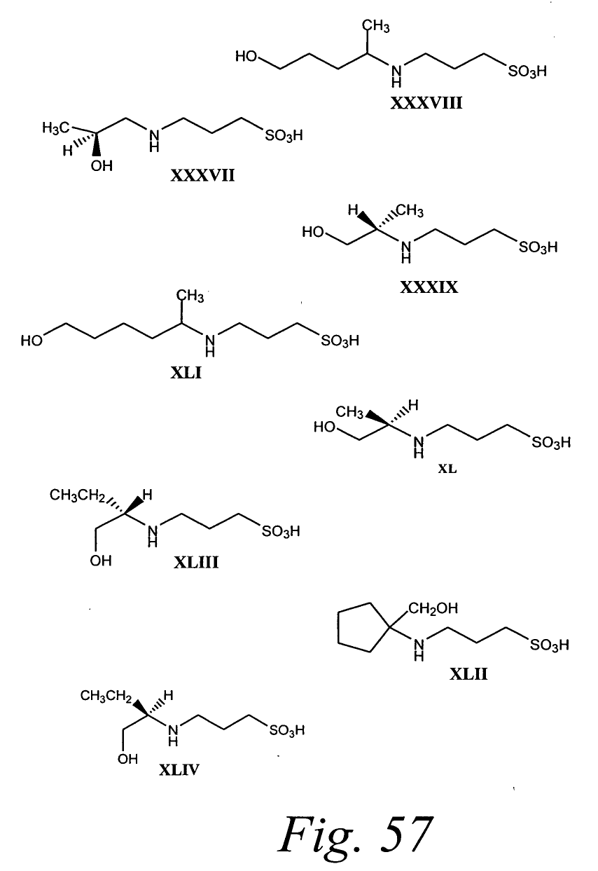

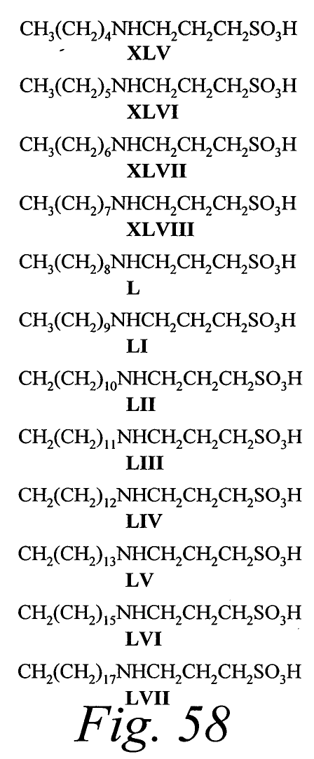

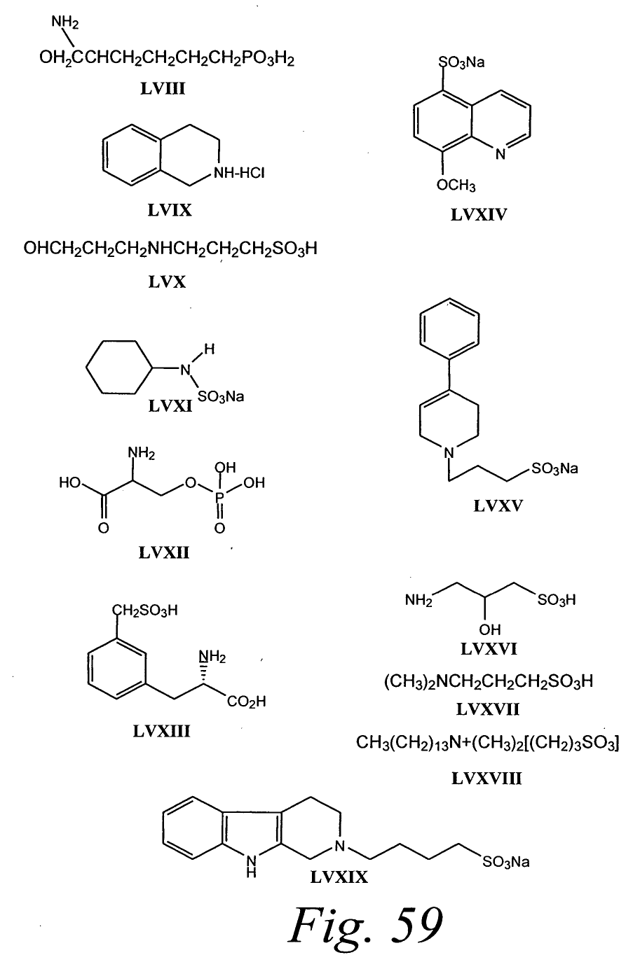

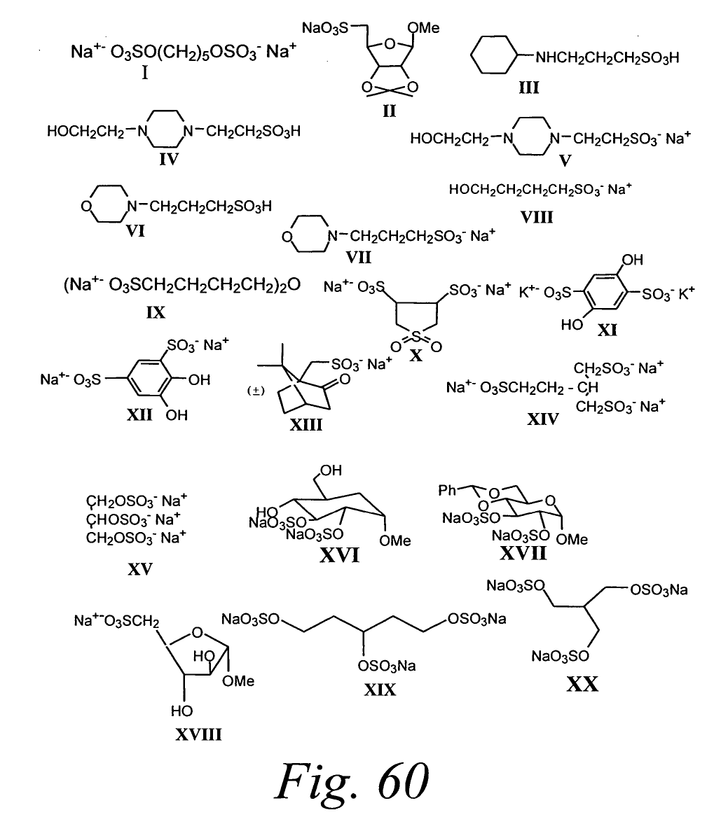

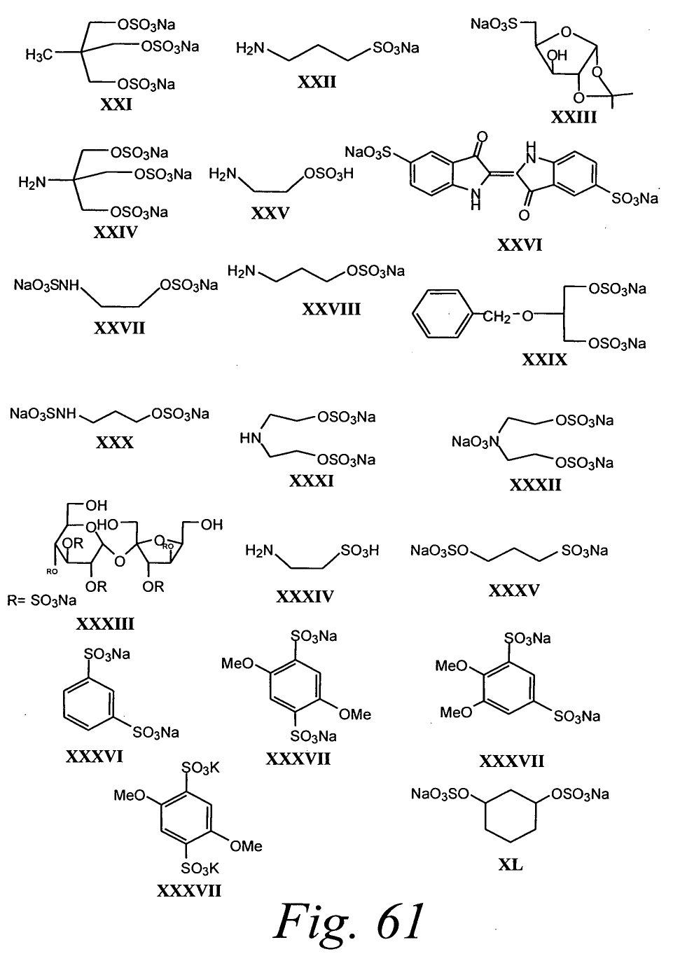

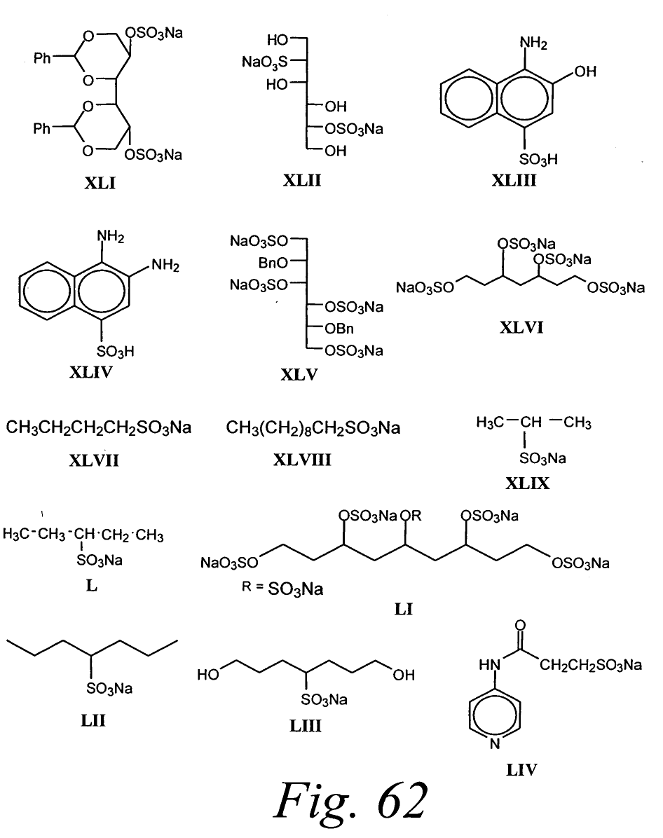

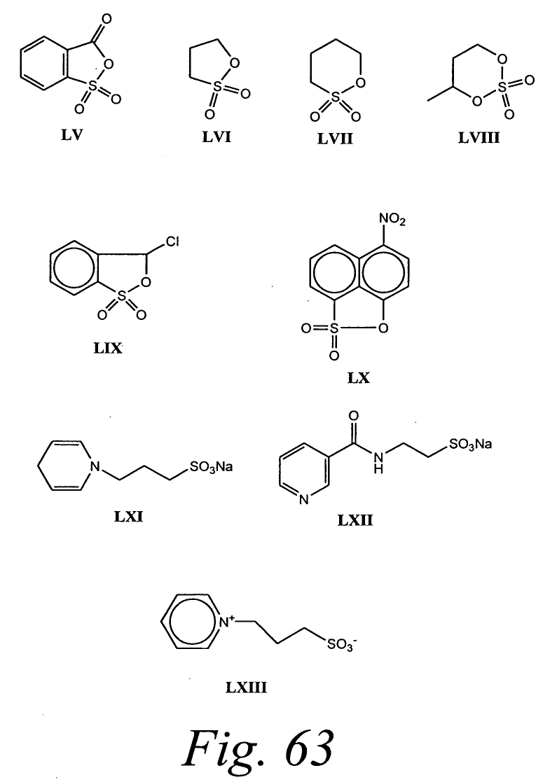

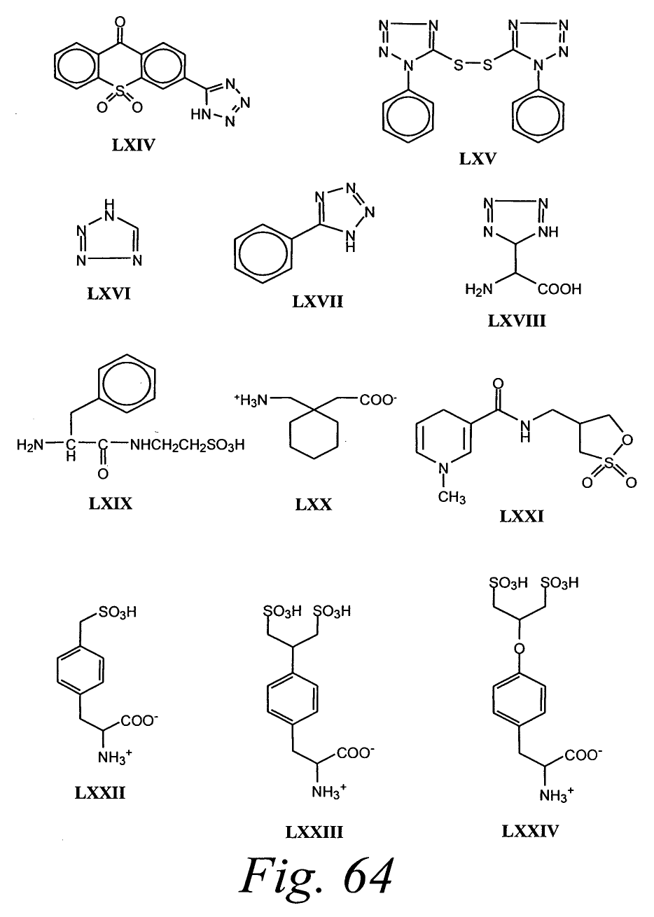

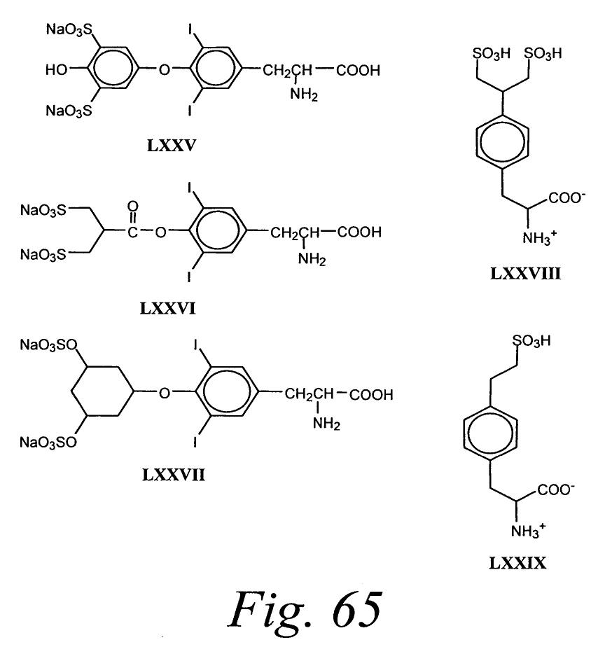

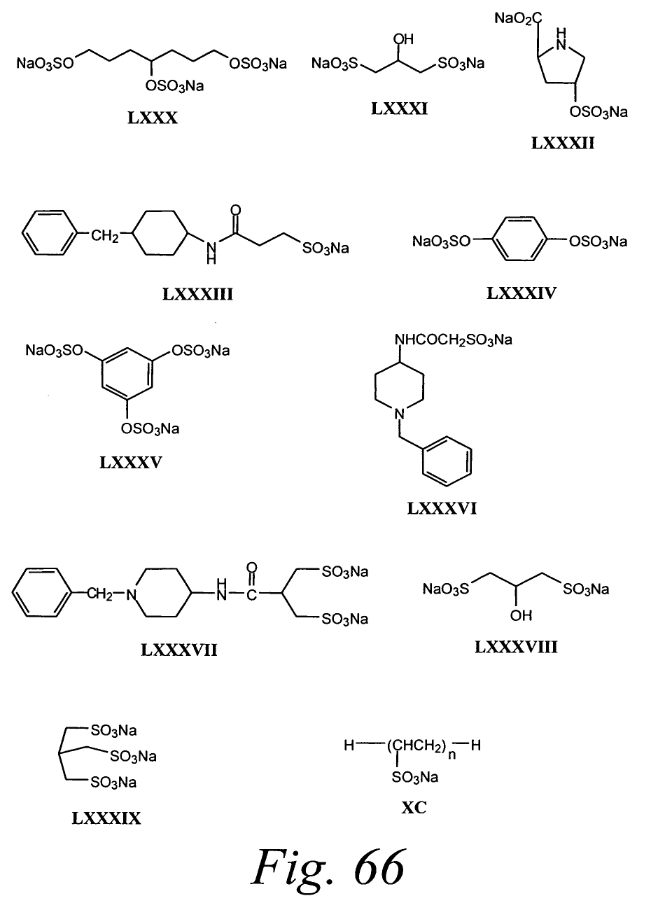

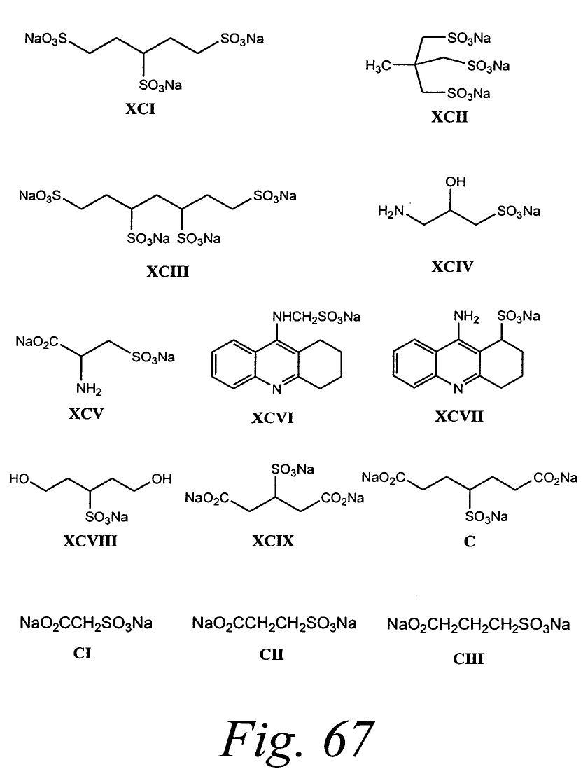

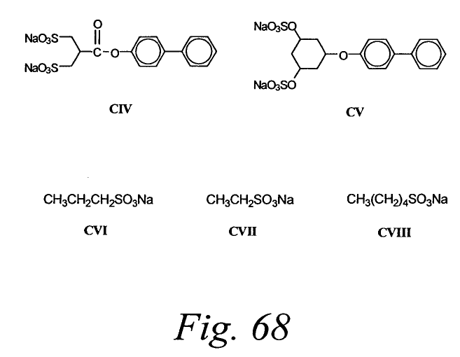

Circular dichroism analysis (FIG. 2) of NAC peptide conformation after 10-72 hours of incubation presented a minimum at 227 nm reminiscent of that observed with regions rich in α-helices. In presence of heparin the CD spectrum of NAC after incubation displayed a clear minimum at 218 nm typical of a β-sheet conformation. The appearance of NAC fibers was followed by Electron Microscopy (FIG. 3). Presence of heparin clearly promotes a conformational change and favors rich β-sheet content which facilitates aggregate/fibril formation (FIG. 2). This result indicates that glycosaminoglycans do participate in the oligomerization/fibril formation process of NAC and validates the approach of using GAG-mimetic compounds as a means to prevent oligomerization of NAC and the formation of toxic aggregates. This observation is supported by EM analysis where NAC peptides appeared to form longer and more interwined fibers in the presence of heparin (FIG. 4).

Results of the CD assay for a number of test compounds are summarized in the Table below.

CD Analysis of Test Compounds

| | CD Result | Activity | Structure |
|---|---|---|---|
| 1 | Random Coil β-sheet | (½) Yes | 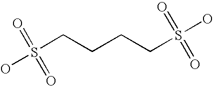 |
| 2 | Random Coil TRansition | ( 2/2) Yes | 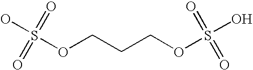 |
| 3 | β- turn/βsheet | ( 0/2) No | 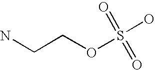 |
| 4 | β-turn | ( 0/2) No | 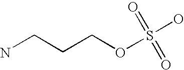 |
| 5 | β-sheet | ( 0/2) No | 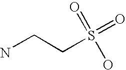 |
| 6 | β-turn transition | (½) Yes | 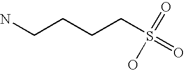 |
| 7 | β- turn/βsheet Random coil Transition | ( 2/4) Yes | 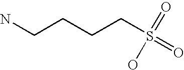 |
| 8 | β-turn/β- sheet | ( 0/2) No | 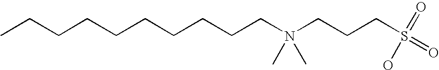 |
| 9 | Random Coil | ( 4/4) Yes | 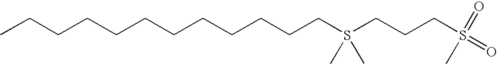 |
| 10 | Random Coil | ( 5/5) Yes | 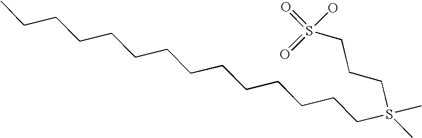 |
| 11 | Random Coil | ( 4/4) Coil | 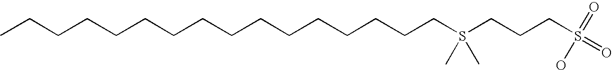 |
| 12 | β-turn Transition | (⅓) No | 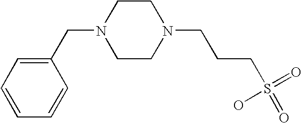 |
| 13 | Random coil Transition | ( 3/3) Yes | 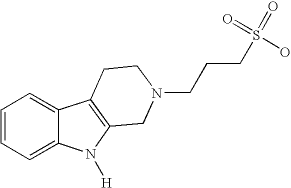 |
| 14 | Random Coil β-sheet/β- turn | ( 2/4) Yes | 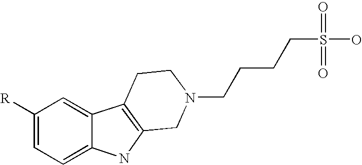 |
| 15 | β-sheet/β- turn Random Coil | (¼) No | 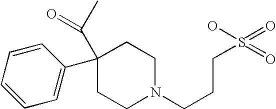 |
| 16 | Random Coil | ( 3/3) Yes | 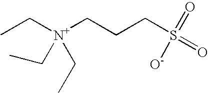 |
| 17 | β-turn Transition | (⅓) No | 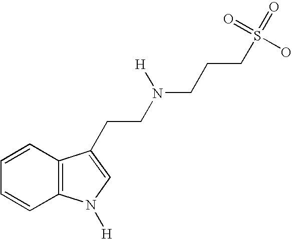 |
| 18 | β-sheet/β- turn | ( 0/2) No | 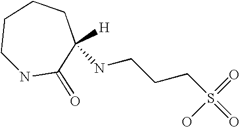 |
| 19 | Random Coil β-turn | (½) Yes | 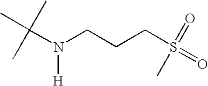 |
| 20 | β-turn | ( 0/2) No | 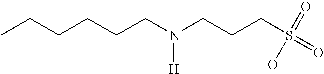 |
| 21 | β-sheet/β- turn Transition | (⅓) No | 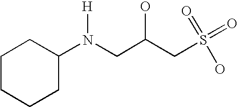 |
| 22 | Transition β-turn | (⅔) No | 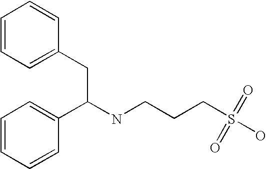 |
| 23 | Random Coil β-sheet | (½) Yes | 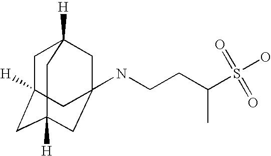 |
| 24 | Random Coil | ( 2/2) Yes | 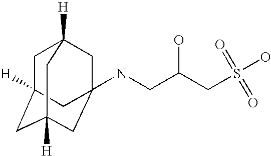 |
| 25 | Random Coil | ( 2/2) Yes | 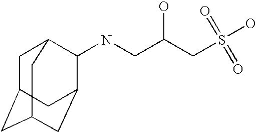 |
| 26 | Transition β-turn | (½) Yes | 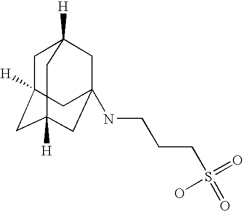 |
| 27 | β-sheet/β- turn | ( 0/2) No | 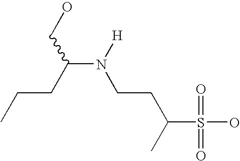 |
| 28 | β-turn/β- sheet | ( 0/3) No | 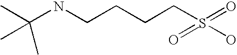 |
| 29 | β-turn | ( 0/3) No | 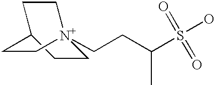 |
| 30 | Random Coil β-sheet/β- turn | ( 2/4) Yes | 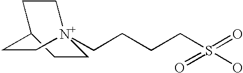 |
| 31 | β-sheet/β- turn α-helix | (⅓) No | 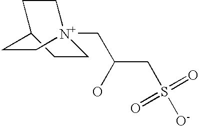 |
| 32 | β-sheet/β- turn Random coil | (¼) No | 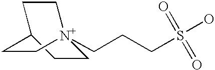 |
| 33 | Random Coil β-sheet/β- turn | (½) Yes | 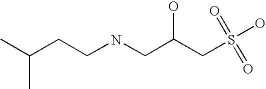 |
| 34 | Random Coil β-sheet | (½) Yes | 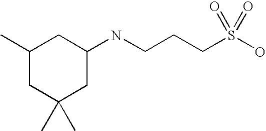 |
| 35 | β-turn | ( 0/2) No | 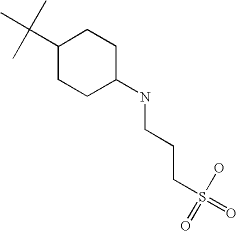 |
| 36 | β-sheet | ( 0/1) No | 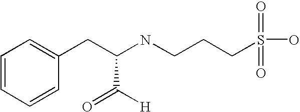 |
| 37 | β-sheet Transition | (½) Yes | 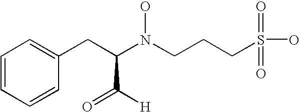 |

When incubated with test compounds, NAC displayed different conformations as observed by CD analysis: “Random Coil” signifies that the NAC in the sample was observed to have been maintained in its original random coil conformation indicating that the compound coincubated with NAC is active; “β-sheet” or “β-sheet/β-turn” signifies that the compound was not able to prevent the shift from random coil to β-sheet; “Transition” signifies that the NAC was observed to be in the process of converting from random coil to β-sheet indicating that the compound was able to slow down the conformational change and indicates activity. For compounds that have been tested multiple times, a compound is classified as active if it prevents the transition to β-turn/β-sheet in 50% or more of the trials.

Analysis by Electron Microscopy: The fibrillogenesis assays performed for CD analysis were also spotted for EM. The electron micrographs are visually examined and a score is assigned as follows. Briefly, 10 representative frames from a 300-mesh grid are analyzed and given a score representing the amount of fibers formed in the sample as compared to a micrograph with NAC alone without a test compound (100% fibers). A score was assigned based on this analysis on a scale from 0% to 100% fibers: (−) indicates an observation of less than about 25% fibers; (+) indicates about 25% fibers; (++) about 50% fibers; (+++) indicates about 75% fibers; and (++++) 100% fibers. (AA) indicates that amorphous aggregates were observed. The results for samples analyzed by EM are detailed in the Table below.

EM results

| Structure | EM Score | Activity |
|---|---|---|
| 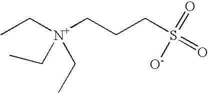 | AA+ | Yes |
| 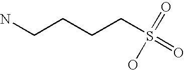 | AA+ | Yes |
| 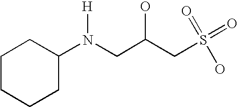 | +++ | No |
| 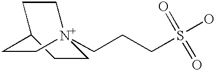 | + | Yes |

### Example 4

### Compounds which Prevent Protein Aggregation Disorders can be Screened in Cellular Models

Various cell culture models have been documented to follow the formation of aggresomes associated with the accumulation of proteins. In addition such proteins oligomerize and aggregate and induce toxicity in these cells in culture. For Parkinson's disease models, overexpression of various synuclein mutants induces the formation of aggresomes in several cell lines: SH-SY5Y cells (Kanda, et al. 2000. Neurosci 97:279), BE(2)-M17 neuroblastoma cells, HEK293 (Ko, et al. 2000. J Neurochem 75:2546), NT-2, SK-N-MC cell lines (Lee, et al. 2001. J Neurochem 76:998) and BE-M17 neuroblastoma cells (Ostrerova-Golts, et al. 2000. J Neurosci 20:6048).

Similar models have been developed to study the formation of aggresomes by other proteins such as huntingtin involved in the development of Huntington's disease; Expression of the mutant huntingtin gene in PC-12 cell line (Wu, et al. 2002. JBC 277:44208; Igarashi, et al. 2003. Mol Neurosci 14:565) or in Cos-7 (Carmichael, et al. 2002. Neurosci Lett 330:270-274; Yang, et al. 2002. Hum Mol Gen 11:2905) leads to aggresomes.

Detection of aggresomes or inclusions can readily be achieved by immuno-colocalization of target aggregated protein (e.g., synuclein or huntingtin) with various. cytoskeletal proteins such as β-tubulin, γ-tubulin or vimentin. The exclusion of other markers (such as α-mannosidase II for the Golgi apparatus) can also further define the cellular distribution of the inclusions. Other markers such as ubiquitin can be useful for the characterization of aggregates which accumulate as aggresomes in the perinuclear area at the microtubule organizing center (MTOC) as previously demonstrated for DPRLA and schwannomin (Shimohata, et al. 2002; Gautreau, et al. 2003; see table above). Other aggregates or inclusions can be found within the cytoplasm (Lewy bodies, Mallory bodies) or the nucleus (huntingtin, ataxins).

Although these large aggregates are visually detectable by microscopic analysis, they may not themselves harbor toxicity, and their presence may not be sufficient to induce toxicity. However, their presence is indicative of the accumulation of abberant protein conformers and assembly intermediates which have been shown to induce cellular toxicity.

Recent evidence clearly demonstrates the toxic effect of androgen-receptor containing expanded polyglutamine tracts responsible for spinobulbar muscular atrophy. Following tranfection of AR mutant with 112 glutamine repeats (AR-112Q) all cells display cytoplamic inclusions typical of aggresomes and display significant cell death (Taylor, et al. 2003. Hum Mol Genet 12:749). Importantly, inhibition of aggresome formation using nocodazole leads to an increase in cell death in a dose-dependent fashion underlining the importance of aggresomes in protecting cells against the toxicity of oligomers/aggregates.

Cells which accumulate aggregates can be treated in vitro using proprietary compounds and the formation of toxic aggregates can be followed using a number of methods. Following the treatment of cells which express mutant proteins, aggregates or inclusions can be quantified by microscopy using fluorescent markers and the viability of cells can be followed using viability assays as discussed above or using MTT or WST-1 staining. (Abee and Matsuke. 2000. Neurosc Res 38:3256; Berridge, et al. 1996. Biochemica 4:11). The amount of cell death can be quantified using FACS-based survival assays (Taylor, et al. 2003. Hum Mol Genet 12: 749).

The characterization and quantification can be furthered by isolating aggresomes or aggregates by cell disruption and centrifugation.

### Example 5

### A Method for Isolating Aggresomes Associated with Protein Aggregation Disorders

Because the aggresome includes a juxtanuclear cap of vimentin, the technique of Starger (Starger and Goldman, 1977. Proc Natl Acad Sci USA 74:2422) can be modified to optimize for the isolation ofisolate protein aggregates from aggresomes as demonstrated for the cystic fibrosis transmembrane regulator (CFTR) (Wanker, et al. 1999. Methods in Enzymology 309:375). In brief, cells are grown to 85% confluency in 100-mm dishes and treated with 10 mg/ml ALLN for 12 h before isolation. Cells are washed twice in PBS (6 mM sodium-potassium phosphate buffer, 170 mM NaCl, 3 mM KCl), scraped, and collected for 3 min at 2,500 g. Washed cells are resuspended in 1 ml PBS per 100-mm plate and passaged through a 25-gauge needle three to four times until bright-field microscopy reveals the majority of cells are disrupted. This material is washed by resuspension and sedimentation at 2,000 g three times in PBS. The resulting material is examined by fluorescence microscopy for the presence of GFP-containing isolated aggresomes. This resulting cellular fraction enriched for aggresomes is collected a final time at 2,000 g and resuspended in 200 ml of PBS/1% BSA. This can serve as the starting material for the immunoelectron microscopy techniques. The formation of these protein aggregates can be further quantified by quantitative ELISA following protein denaturation or dot blot filtration- retardation assay following the isolation of the aggregates by centrifugation as described below (Johnston, et al. 1998. JCB 143:1883).

### Example 6

### Dot-Blot Filter Retardation Assay

Cells expressing a mutated protein are washed in ice-cold phosphate-buffered saline (PBS), scraped, and pelleted by centrifugation (2000 g, 10 min. at 4° C.). Cells are lysed on ice for 30 min. in 500 μl lysis buffer [50 mM Tris-HCl (pH 8.8), 100 mM NaCl, 5 mM MgCl2, 0.5% (w/v) Nonidet P-40 (NP-40), 1 mM EDTA] containing protease inhibitors PMSF (2 mM), Leupeptin (10 μg/ml), pepstatin (10 μg/ml), aprotinin (1 μg/ml) and antipain (50 μg/ml). Insoluble material is removed by centrifugation for 5 min. at 14000 rpm in a microfuge at 4° C. Pellets containing the insoluble material are resuspended in 100 μl DNase buffer [20 mM Tris-HCl (pH 8.0), 15 mM MgCl2] and DNase I (Boehringer Mannheim) is added to a final concentration of 0.5 mg/ml followed by incubation at 37° C. for 1 hour. After DNase treatment the protein concentration is determined by the dot metric assay (Geno technology) using BSA as a standard. Incubations are terminated by adjusting the mixtures to 20 mM EDTA, 2% (w/v) SDS, and 50 mM DTT, followed by heating at 98° C. for 5 min.

The filter assay used to detect polyglutamine-containing huntingtin protein aggregates has been described (Johnson, et al. 1995. J Mol Recog 8:125). Denatured and reduced protein samples are prepared as described above, and aliquots corresponding to 50-250 ng fusion protein or 5-30 μg extract protein (pellet fraction) of transfected cells are diluted into 200 μl 2% SDS and filtered on a BRL dot-blot filtration unit through a cellulose acetate membrane (Schleicher and Schuell, Keene, N.H., 0.2 μm pore size) that has been preequilibrated with 2% SDS. Filters are washed twice with 200 μl 0.1% SDS and are then blocked in TBS (100 mM Tris-HCl, pH 7.4, 150 mM NaCl) containing 3% nonfat dried milk, followed by incubation with the anti-HDl antibody (1:1000). The filters are washed several times in TBS and are then incubated with a secondary anti-rabbit antibody conjugated to horseradish peroxidase (Sigma, 1:5000) followed by ECL (enhanced chemiluminescence, Amersham) detection. The developed blots are exposed for various times to Kodak (Rochester, NY) X-OMAT film or to a Lumi-Imager (Boehringer Mannheim) to enable quantification of the immunoblots.

For detection and quantification of polyglutamine-containing aggregates generated from the protease-treated fusion proteins with GST-x, the biotin/streptavidin-AP detection system is used. Following filtration, the cellulose acetate membranes are incubated with 1% (w/v) BSA in TBS for 1 hr at room temperature with gentle agitation on a reciprocal shaker. Membranes are then incubated for 30 min with streptavidin-alkaline phosphatase (Promega, Madison, Wis.) at a 1:1000 dilution in TBS containing 1%. BSA, washed three times in TBS containing 0.1% (v/v) Tween 20 and three times in TBS, and finally incubated for 3 min with either the fluorescent alkaline phosphatase substrate AttoPhos or the chloro-substituted 1,2-dioxetane chemiluminescence substrate CDPStar (Boehringer Mannheim) in 100 mM Tris-HCI, pH 9.0, 100 mM NaCl, and 1 mM MgCl₂. Fluorescent and chemiluminescent signals are imaged and quantified with the Boehringer Lumi-Imager Fl system and LumiAnalyst software (Boehringer Mannheim).

### Example 7

### Filter Trap Assay

The filter trap assay is used to detect the presence of aggregates in a simple assay starting from frozen brain samples. The method can be modified for the detection of various types of protein aggregates. Frozen mouse hemi-brains or pieces of frozen human brain are weighed and homogenized with a polytron in 10 volumes of phosphate-buffered saline (pH, 7.4) containing 1×protease inhibitor cocktail (Cat. #P8340, Sigma, St. Louis, Mo.). Homogenates are centrifuged at 3,000 rpm for 5 min at 4° C. in a microcentrifuge. The supernatant is aliquoted, and the protein concentration is determined by the BCA method (Pierce Chemical Co., Rockford, Ill.). Aliquots are frozen at −70° C. until use. Before filtering, the samples are thawed and diluted with PBS to a final volume of 200 μL containing 1% SDS (except in FIG. 2, where the final SDS concentration varied between 0.1 and 5%). The solution is then passed through cellulose acetate membranes, 0.2-μm pore size (OE66, Schleicher & Schuell, Keene, N.H.), using a 96-well dot-blot apparatus (Bio-Rad Laboratories, Hercules, Calif.). Before filtration, the membranes are immersed in PBS containing 1% SDS. Filter dots are washed twice with 500 μL of PBS (pH, 7.4). Proteins trapped by the filter are detected by immunostaining following protocols used in immunoblotting (Xu, et al. 2002 Alzheimer Dis Assoc Disord 16:191)

To analyze the proteins trapped by filtration, the target dots are cut out and boiled in 40 μL 1×SDS loading buffer (Laemmli, 1970) for 10 min and then mixed vigorously (by vortex). Samples (20 μL) are loaded on SDS/polyacrylamide gel and then blotted onto nitrocellulose membrane (BA-S85, Schleicher & Schuell, Keene, N.H.). Immobilized proteins are detected with mAb 6E10 and ECL (NEN Life Science Products, Inc., Boston, Mass.), as previously described (Jankowsky et al., 2001).

### Example 8

### Methodology used to Detect a Plurality of Detrimental Protein Aggregates Associated with Protein Aggregation Disorders

The following Table outlines references that detail methods and techniques to detect inclusions or aggresomes associated with various diseases. It should be noted that the techniques described hereincan be used by one skilled in the art to practice the methods described herein.

| | | | Inclusion | |
|---|---|---|---|---|
| Study | Aggresome detection | | type & | |
| Proteins | technique | Indication | localisation | Reference |
| Parkin | 1 - Colocalization by | Parkinson's | Lewy bodies | Junn et al., |
| α-synuclein | Immunofluorescence | disease | cytoplasmic | 2002. JBC 277, |
| synphilin-1 | with Parkin/γ-tubulin | | | 47870-47877 |
| | (MOTC marker) | | | |
| | 2 - Microtubule inhibitors/ | | | |
| | proteasome inhibitors | | | |
| DRPLA | 1 - Colocalization by | Dentatorubral- | Aggresomes | Shimohata et |
| protein | Immunofluorescence | pallidoluysian | Perinuclear | al. 2002., |
| | with β-tubulin/γ-tubulin/ | atrophy | & nuclear | Neurosci.Lett. |
| | vimentin | | inclusions | 323, 215-218 |
| | 2 - Microtubule inhibitors/ | | | |
| | proteasome inhibitors | | | |
| Huntingtin | Colocalization by | Huntington's | Intranuclear | Waelter et al., |
| | Immunofluorescence | disease | inclusions & | 2001. |
| | with γ-tubulin/vimentin | | Dystrophic | Mol.Biol.Cell |
| | | | neurites or | 12, 1393-1407 |
| | | | neuropil | |
| | | | cytoplasmic | |
| | | | aggregates | |
| Cystic fibrosis | 1 - Colocalization by | Cystic fibrosis | Aggresomes, | Johnston et |
| transmembrane | Immunofluorescence | | Cytoplasmic | al, 1998, JCB |
| regulator | with vimentin/γ-tubulin | | | 143, 1883-1898 |
| (CFTR) | 2 - proteasome inhibitor | | | |
| | 3 - Electron microscopy | | | |
| | 4 - Subcellular | | | |
| | fractionation and | | | |
| | aggresome isolation | | | |
| α-synuclein | Colocalization by | Parkinson's | Lewy bodies | McNaught et al |
| | Immunofluorescence | disease | cytoplasmic | 2003. |
| | with γ-tubulin | | | Eur.J. |
| | | | | Neurosci. 16, |
| | | | | 2136-2148 |
| Schwannomin | 1 - Colocalization by | Neurofibromatosis | Aggresomes | Gautreau et al |
| | Immunofluorescence | type 2 | cytoplasmic | 2003. |
| | with γ-tubulin | | | JBC 278, 6235-6242 |
| | 2 - Nocodazole | | | |
| | 3 - EM | | | |
| | 4 - Pulse | | | |
| | chase/immunoprecipitation | | | |
| | of components | | | |
| cytokeratin | Colocalization by | alcoholic | Mallory body | French et al., |
| | Immunofluorescence | hepatitis | | 2001. Exp. |
| | with ubiquitin/ | nonalcoholic | | Mol. Pathol. |
| | proteasome subunit P25 | steatohepatitis, | | 71, 241-246 |
| | | chronic | | |
| | | cholestasis, | | |
| | | copper | | |
| | | toxicity, drug | | |
| | | toxicity, and | | |
| | | hepatocellular | | |
| | | neoplasms. | | |

### Example 9

### Compound Screening in Transgenic Mouse Models of Protein Aggregation Disorders

Examples of transgenic mouse lines developed to model human proteopathies are provided in the Tables below.

Constructs for the expression of various mutant proteins have been designed and introduced in transgenic animals to model various Protein Aggregation Disorders or Proteophathies. The expression of these genes leads to the development of various neuropathological and behavioral changes consistent with the human condition associated with the mutant gene.

For example, transgenic mice expressing modest levels of the long isoform of Tau bearing mutations found in frontotemporal dementia and parkinsonism patients, develop a tauopathy characterized by congophilic hyperphosphorylated tau inclusions in forebrain neurons. These inclusions appear as early as 18 months of age. As with human cases, tau inclusions were composed of both mutant and endogenous wild-type tau, and are associated with microtubule disruption and flame-shaped transformations of the affected neurons. Behaviorally, aged Tg Tau R406W mice displayed cognitive deficits and in particular associative memory impairment (See Table).

Another example of the successful modeling of human disease is the transgenic mouse model expressing alpha-synuclein mutations, A30P and A53T. These mice develop early onset progressive decline of motor function. Neuropathologically these animals display typical alpha-synuclein immunoreactive Lewy body inclusions in the neurites (see Table).

Various huntingtin alleles have also been introduced in the mouse. Transgenic mice expressing huntingtin alleles with a diverse set of expanded polyglutamine stretches develop early clasping phenotype, motor coordination impairment and hyperactivity. This disorder is associated with neuropathological appearance of cortical, septal, hippocampal inclusions, reactive gliosis, cell loss, general brain atrophy and cellular inclusions consistent with changes normally seen in Huntington's patients (see Table).

Therefore, the use of such transgenic mouse models exhibiting phenotypes mimicking various human non-amyloid protepathies provides animal models for testing the therapeutic efficacy of compounds which can bind to the target proteins, prevent their assembly, oligomerization, aggregation or facilitate their clearance.

| Mutation/ | Transgene/ | Genetic | Behavioural | Neurological | |
|---|---|---|---|---|---|
| Gene | Promoter | Background | Phenotype | Characteristics | Citation |
| Tau P301L | Tau with 4 | C57BL6 | Early and | Fibrillary gliosis in | Lewis J et al. 2000. |
| | repeats, and | DBA/2 SW | severe motor | the anterior horns, | Nat Genet25: 402-5. |
| | exon 10 but with | | and | axonal | |
| | exons 2 and 3 | | behavioral | degeneration, | |
| | deletions and | | deficits | neuronal lesions | |
| | P301L mutation/ | | | | |
| | PrP promoter | | | | |
| Tau P301L | Tau40 isoform | B6D2F1, | Signs of | Numerous tau- | Götz J et al. 2001. J |
| | with 4 repeats, | C57BL/6 | Wallerian | reactive nerve cell | Biol Chem. 276: 529-34. |
| | exons 2 and 3 | | degeneration, | bodies and dendrites; | |
| | and P301L | | neurogenic | large numbers of | |
| | mutation/ | | muscle | pathologically | |
| | Thy1.2 | | atrophy, | enlarged axons with | |
| | promoter | | muscle | neurofilaments and | |
| | | | weakness. | tau-reactive | |
| | | | | spheroids | |
| Tau | Tau with R406W | B6SJL/F1 | Impaired | Accumulation of | Tatebayashi Y et al. |
| R406W | mutation/ | C57BL/6J | associative | insoluble tau. | 2002. Proc Natl Acad |
| | αCaMk-II | | memory. | Congophilic | Sci USA. 99: 13896-901. |
| | promoter | | Abnormal | hyperphosphorylated | |
| | | | prepulse | tau inclusions in | |
| | | | inhibition and | forebrain neurons. | |
| | | | forced swim | | |
| | | | test. | | |
| Tau G272V | Tau40 with | B6D2F1 | No | Filaments in | Götz J et al. 2001. |
| | G272V | C57BL/6 | neurological | oligodendrocytes, | Eur J Neurosci. |
| | mutation/prion | | deficits | with phosphor-tau | 13: 2131-40 |
| | protein promoter | | | phosphorylation. In | |
| | | | | the spinal cord, | |
| | | | | ThioS-positive | |
| | | | | fibrillary inclusions | |
| | | | | in oligodendrocytes | |
| | | | | and motor neurons | |
| Tau | Tau with V337M/ | B6SJL | High | Irregularly shaped | Tanemura K et al. |
| V337M | PDGF-b | | locomotion. | neurons in | 2002. J Neurosci. |
| | promoter, | | Significant | hippocampus were | 22: 133-41. |
| | Thy1.2 | | difference in | immunoreactive for | |
| | promoter | | elevated plus | Tau and containeind | |
| | | | maze test and | paired helical | |
| | | | conditional | filament. Atrophic | |
| | | | fear test. | cell death. | |
| Tau 3- | Tau with 3 | B6D2/F1 | Progressive | Accumulation of | Ishihara T et al. 1999. |
| repeats | repeats,/ | | motor | insoluble tau, | Neuron 24: 751-62 |
| | Prion Protein | | weakness, | profound astrocytosis | |
| | promoter, | | impaired | and axonal | |
| | | | coordination | degeneration. | |
| Tau 4- | Tau with 4 | FVB/N | Motor and | Axonal degeneration, | Spittaels K et al. 1999 |
| repeats | repeats/mouse | | sensory | astrogliosis and | Am J Pathol. 155: |
| | thy-1 promoter | | deficiencies. | ubiquitination of | 2153-65 |
| | | | | accumulated proteins | |
| Tau 4- | Tau with 4 | B6D2F1 x | Motor and | Prominent | Probst A et al. 2000 |
| repeats | repeats/mouse | B6D2F1 | reflex | somatodendritic | Acta Neuropathol. |
| ALZ17 | Thy.1.2 | C57BL/6 | deficiencies. | staining | 99: 469-81 |
| | promoter | | | ofhyperphosphorylated | |
| | | | | tau with pominent | |
| | | | | axonopathy | |

| Gene/ | Transgene/ | Genetic | Behavioural | Neurological | |
|---|---|---|---|---|---|
| Mutations | Promoter | Background | Phenotype | Characteristics | Citation |
| N171HD | cDNA encoding an | C3H | Behavioural | Inclusions in | Schilling G et al |
| | N-terminal fragment | C57BL/6 | abnormalities | striatum, cortex, | 1999. Human |
| | (171 amino acids) of | | appear only in | hippocampus, | Molecular |
| | huntingtin with 82, 44 | | 82Q present | amygdala and | Genetics 8: |
| | or 18 glutamines/ | | loss of | cerebellum. | 397-407. |
| | prion protein | | coordination, | Diffuse nuclear | |
| | promoter | | tremors, | accumulation of | |
| | | | hypokinesis and | htt protein. | |
| | | | abnormal gait. | Striatal cell loss | |
| | | | Early clasping | and overall brain | |
| | | | and motor | atrophy. | |
| | | | coordination | | |
| | | | impaired . . . | | |
| | | | Premature | | |
| | | | death. | | |
| HD | HD gene with either | FVB/N | Ccircling, | Iinclusions and | Reddy PH et al. |
| | 16, 48 or Q89 | | hyperactive | gliosis in | 1998. Nature |
| | glutamine repeats | | Early clasping | striatum, cerebral | Gen. 20: 198-202. |
| | CMV promoter | | phenotype in | cortex, thalamus, | |
| | | | mice with 89Q | hippocampus. | |
| | | | and 48Q. | Striatal cell loss. | |
| HD | HD gene exons 1-3 of | FVB/N | Prolonged | Inclusion | |
| | with 89Q repeats | | hyperactivity | throughout the | |
| | | | and early | brain. | |
| | | | clasping. | | |
| HD | HD gene with 100Q, | SJL | Hyperactivity. | Neuronal | DiFiglia et al. |
| | 48Q or 18Q repeats/ | C57BL/6 | 100Q mice | intranuclear | 1997 Science |
| | NSE promoter | | show early | inclusions and | 277: 1990-1993. |
| | | | clasping | dystrophic | |
| | | | phenotype and | neurites in cortex | |
| | | | motor | and striatum. | |
| | | | coordination | Cell loss and | |
| | | | impaired | brain atrophy. | |
| Conditional | Conditional | CBA | Late onset | Inclusions in | Yamamoto et |
| HD mutant | expression (tet-off; | C57BL/6 | tremors and | striatum, septum, | al. 2000. Cell |
| | CamKIIα-tTA) of a | | abnormal gait. | cortex, hippocam | 101: 57-66. |
| | mutated huntingtin | | Early clasping | pus. Reactive | |
| | protein with 94Q | | and motor | astrocytes. | |
| | | | coordination | Progressive | |
| | | | impaired | overall brain | |
| | | | | atrophy. | |

| Gene/ | Transgene/ | Genetic | Behavioral | Neurological | |
|---|---|---|---|---|---|
| Mutations | Promoter | Background | Phenotype | Characteristics | Citation |
| Alpha | α-synuclein | C57BL/6 | Early | Astrocytic gliosis and | Putten H et |
| Synuclein | with A53T/ | | progressive | microglial activation. | al. 2000. J |
| | Thy1 gene | | motor function | Diffuse perikaryal a- | Neurosci 20: |
| | promoter | | impairment. | synuclein staining. Intense | 6021-9. |
| | | | | staining with the Campbell- | Sommer B et |
| | | | | Switzer pyridine silver, | al. 2000 |
| | | | | showing Lewy-type changes | Exp Gerontol. |
| | | | | | 35: 1389-403. |
| Alpha- | α-synuclein - | C57BL/6 | No motor | Alpha-syn positive neurites | Kahle PJ et |
| Synuclein | with A30P/ | | abnormalities | exhibit Lewy body | al. 2002 |
| A30P | Thy1 promoter | | | charactersitics, emanating | J Clin. Invest |
| | | | | from neuronal cell bodies | 110: 1429-1439. |
| Alpha | wt α-synuclein | C57BL/6 | None listed. | Abnormal Tg α-syn-positive | Kahle et al. |
| Synuclein | | | | neurites, characteristic of | 2001. Am J |
| | | | | Lewy body disease, | Pathol. 159: |
| | | | | | 2215-25. |
| Alpha | α-synuclein | Tg5093 | progressive | No discrete Lewy body-like | Gomez-Isla et |
| synuclein | A30P or A53T | | motor disorder | α-synuclein inclusions could | al., 2003. |
| A30P or | | | with rigidity, | be identified. No specific | Neurobiology |
| A53T | | | dystonia, gait | deterioration of the | of Aging 24: |
| | | | impairment, | nigrostriatal dopaminergic | 245-258. |
| | | | and tremor. | system. | |
| Alpha | α-Synuclein. | C3H/HeJx | Adult-onset | Neuronal abnormalities in | Lee et al., |
| synuclein | A30P or A53T | C57BL6/J | neurodegenerative | perikarya and neurites | 2002. Proc |
| | | | disease | including pathological | Natl Acad Sci |
| | | | with | accumulations of | USA. |
| | | | progressive | α-Syn and ubiquitin. | 99: 8968-73 |
| | | | motor | | |
| | | | dysfunction. | | |
| APP + Alpha | Heterozygous | | | | Masliah E et |
| Synuclein | α-SYN mice | | | | al. 2001. Proc |
| | from line D | | | | Nat Acad Sci |
| | were crossed | | | | USA 98: |
| | with | | | | 12245-50. |
| | heterozygous | | | | |
| | hAPP mice | | | | |
| | from line J9 | | | | |

### Example 11

### Evaluation of Compounds Binding to NAC Peptide by Mass Spectrometry

The ability of the compounds of the present invention to bind to NAC peptide in aqueous solution was evaluated. The binding ability correlates to the intensities of the peptide-compound complex peaks observed by the Electrospray Mass Spectrum. Millipore distilled deionized water was used to prepare all aqueous solutions. For pH determination a Beckman (Φ36 pH meter fitted with a Corning Semi-Micro Combination pH Electrode was employed.

NAC (MW 3260.6 Da) at 20 μM was first analyzed at pH 7.40 and the usual sodium clusters was observed at +2, +3 and +4 at m/z 1335.5, 1116.7 and 843.4 respectively. The optimal cone voltage was determined to be 20V.

Mass spectrometry: Mass spectrometric analysis was performed using a Waters ZQ 4000 mass spectrometer equipped with a Waters 2795 sample manager. MassLynx 4.0 (earlier by MassLynx 3.5) was used for data processing and analysis. Test compounds were mixed with disaggregated peptides in aqueous media (6.6% EtOH) at a 5:1 ratio (20 μM NAC: 100 μM of test compound or 40 μM NAC : 200 μM of test compound). The pH of the mixture was adjusted to 7.4 (±0.2) using 0.1% NaOH (3-5 μL). Periodically, NAC peptide solution at 20 μM or 40 μM was also prepared in the same fashion and run as control. The spectra were obtained by introducing the solutions to the electrospray source by direct infusion using a syringe pump at a flow rate of 25 μl/min, and scanning from 100 to 2100 Da in the positive mode. The scan time was 0.9 sec per scan with an inter-scan delay of 0.1 sec and the run time was 5 min for each sample. All the mass spectra were sum of 300 scans. The desolvation and source temperature was 70° C. and the cone and capillary voltage were maintained at 20 V and 3.2 kV respectively.

The total area under the peaks for the bound NAC-compound complex divided by total area under the peaks for unbound NAC was determined for each compound tested. The results are summarized in the Table below.

NAC Peptide Binding Data

| | | Binding |
|---|---|---|
| No. | Structure | Strength* |
| 1 | NaO₃SCH₂(CH₂)₂CH₂SO₃Na | − |
| 2 | NaO₃SOCH₂CH₂CH₂OSO₃Na | − |
| 3 | NH₂CH₂CH₂OSO₃H | − |
| 4 | H₂NCH₂CH₂CH₂OSO₃Na | ++ |
| 5 | H₂NCH₂CH₂SO₃H | + |
| 6 | CH₃(CH₂)₉N⁺(CH₃)₂[(CH₂)₃SO₃⁻] | +++ |
| 7 | CH₃(CH₂)₁₁N⁺(CH₃)₂[(CH₂)₃SO₃⁻] | ++ |
| 8 | CH₃(CH₂)₁₃N⁺(CH₃)₂[(CH₂)₃SO₃⁻] | ++ |
| 9 | 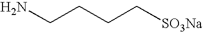 | − |
| 10 | 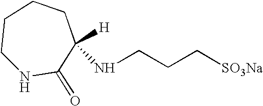 | + |
| 11 | 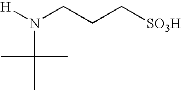 | ++ |
| 12 | 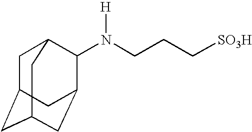 | ++ |
| 13 | 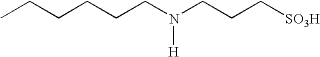 | +++ |
| 14 | 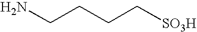 | + |
| 15 | 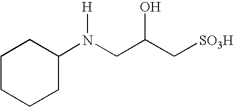 | +++ |
| 16 | 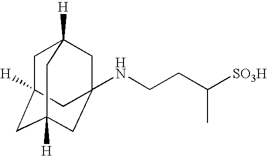 | Indeterminant |

*+++ = Strong; when the total binding is 120% and higher
++ = Moderate; when the total binding is between 120% and 70%
+ = Weak; when the total binding is between 70% and 30%
− = None; when the total binding is between 30% and 0%

1. A method for treating or preventing a Protein Aggregation Disorder in a subject comprising
administering to said subject having a Protein Aggregation Disorder an effective amount of a compound, wherein said compound has one of the following Formulae:
Q—[—Y⁻—X⁺]ₙ
wherein Q is a carrier molecule; Y⁻ is SO₃⁻X⁺, OSO₃⁻X⁺, or SSO₃⁻X⁺; X⁺ is a cationic group; and n is an integer selected such that the biodistibution of the therapeutic compound for an intended target site is not prevented while maintaining activity of the compound or 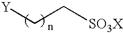
Y is either an amino group or a sulfonic acid group, n is an integer from 1 to 5, and X is hydrogen or a cationic group; or 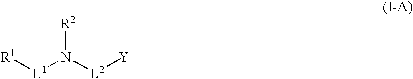
wherein R¹ is a substituted or unsubstituted cycloalkyl, aryl, arylcycloalkyl, bicyclic or tricyclic ring, a bicyclic or tricyclic fused ring group, or a substituted or unsubstituted C₂-C₁₀ alkyl group; R² is selected from the group consisting of hydrogen, alkyl, mercaptoalkyl, alkenyl, alkynyl, cycloalkyl, aryl, arylalkyl, thiazolyl, triazolyl, imidazolyl, benzothiazolyl, and benzoimidazolyl; Y is SO₃⁻X⁺, OSO₃⁻X⁺, or SSO₃⁻X⁺; X⁺ is hydrogen, a cationic group, or an ester-forming group; and each of L¹ and L² is independently a substituted or unsubstituted C₁-C₅ alkyl group or absent, or a pharmaceutically acceptable salt thereof, provided that when R¹ is alkyl, L¹ is absent; or 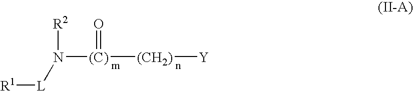
wherein R¹ is a substituted or unsubstituted cyclic, bicyclic, tricyclic, or benzoheterocyclic group or a substituted or unsubstituted C₂-C₁₀ alkyl group; R² is hydrogen, alkyl, mercaptoalkyl, alkenyl, alkynyl, cycloalkyl, aryl, arylalkyl, thiazolyl, triazolyl, imidazolyl, benzothiazolyl, benzoimidazolyl, or linked to R¹ to form a heterocycle; Y is SO₃⁻X⁺, OSO₃⁻X⁺, or SSO₃⁻X⁺; X⁺ is hydrogen, a cationic group, or an ester forming moiety; m is 0 or 1; n is 1, 2, 3, or 4; L is substituted or unsubstituted C₁-C₃ alkyl group or absent, provided that when R¹ is alkyl, L is absent; or 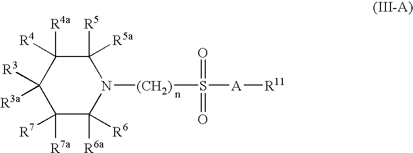
wherein A is nitrogen or oxygen; R¹¹ is hydrogen, salt-forming cation, ester forming group, —(CH₂)ₓ—Q, or when A is nitrogen, A and R¹¹ taken together may be the residue of a natural or unnatural amino acid residue or a salt or ester thereof; Q is hydrogen, thiazolyl, triazolyl, imidazolyl, benzothiazolyl, or benzoimidazolyl; x is 0, 1, 2, 3, or 4; n is 0, 1 2, 3, 4, 5, 6, 7, 8, 9, or 10; R³, R^{3a}, R⁴, R^{4a}, R⁵, R^{5a}, R⁶, R^{6a}, R⁷ and R^{7a} are each independently hydrogen, alkyl, mercaptoalkyl, alkenyl, alkynyl, cycloalkyl, aryl, alkylcarbonyl, arylcarbonyl, alkoxycarbonyl, cyano, halogen, amino or tetrazolyl, or two R groups on adjacent ring atoms taken together with the ring atoms form a double bond, provided that one of R³, R^{3a}, R⁴, R^{4a}, R⁵, R^{5a}, R⁶, R^{6a}, R⁷ and R^{7a} is a moiety of Formula IIIa-A: 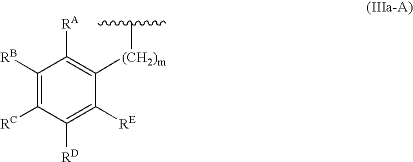
wherein m is 0, 1, 2, 3, or 4; R^{A}, R^{B}, R^{C}, R^{D}, and R^{E} are independently selected from a group of hydrogen, halogen, hydroxyl, alkyl, alkoxyl, halogenated alkyl, mercaptoalkyl, alkenyl, alkynyl, cycloalkyl, aryl, cyano, thiazolyl, triazolyl, imidazolyl, tetrazolyl, benzothiazolyl, and benzoimidazoly; and pharmaceutically acceptable salts and esters thereof, provided that said compound is not 3-(4-phenyl-1, 2, 3, 6-tetrahydro-1-pyridyl)-1-propanesulfonic acid; or 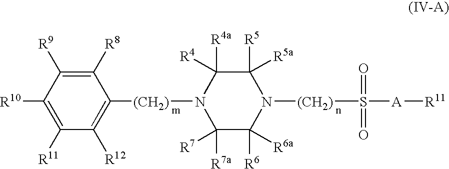
wherein A is nitrogen or oxygen; R¹¹ is hydrogen, salt-forming cation, ester forming group, —CH₂)ₓ—Q, or when A is nitrogen, A and R¹¹ taken together may be the residue of a natural or unnatural amino acid residue or a salt or ester thereof; Q is hydrogen, thiazolyl, triazolyl, imidazolyl, benzothiazolyl, or benzoimidazolyl; x is 0, 1, 2, 3, or 4; n is 0, 1, 2, 3, 4, 5, 6, 7, 8, 9, or 10; R⁴, R^{4a}, R⁵, R^{5a}, R⁶, R^{6a}, R⁷, and R^{7a} are each independently hydrogen, alkyl, mercaptoalkyl, alkenyl, alkynyl, cycloalkyl, aryl, alkylcarbonyl, arylcarbonyl, alkoxycarbonyl, cyano, halogen, amino, tetrazolyl, R⁴ and R⁵ are taken together, with the ring atoms they are attached to, form a double bond, or R⁶ and R⁷ are taken together, with the ring atoms they are attached to, form a double bond; m is 0, 1, 2, 3, or 4; R⁸, R⁹, R¹⁰, R¹¹, and R¹² are independently selected from a group of hydrogen, halogen, hydroxyl, alkyl, alkoxyl, halogenated alkyl, mercaptoalkyl, alkenyl, alkynyl, cycloalkyl, aryl, cyano, thiazolyl, triazolyl, imidazolyl, tetrazolyl, benzothiazolyl, and benzoimidazolyl; or 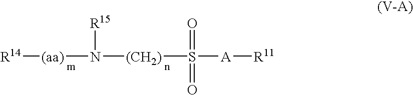
wherein A is nitrogen or oxygen; R¹¹ is hydrogen, salt-forming cation, ester forming group, —(CH₂)ₓ—Q, or when A is nitrogen, A and R¹¹ taken together may be the residue of a natural or unnatural amino acid residue or a salt or ester thereof; Q is hydrogen, thiazolyl, triazolyl, imidazolyl, benzothiazolyl, or benzoimidazolyl; x is 0, 1, 2, 3, or 4; n is 0, 1 ,2 ,3, 4, 5, 6, 7, 8, 9, or 10; aa is a natural or unnatural amino acid residue; m is 0, 1, 2, or 3; R¹⁴ is hydrogen or protecting group; R¹⁵ is hydrogen, alkyl or aryl; or 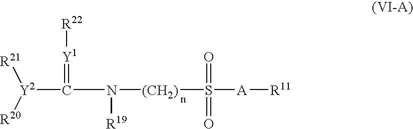
wherein n is 1, 2, 3, 4, 5, 6, 7, 8, 9, or 10; A is oxygen or nitrogen; R¹¹ is hydrogen, salt-forming cation, ester forming group, —(CH₂)ₓ—Q, or when A is nitrogen, A and R¹¹ taken together may be the residue of a natural or unnatural amino acid residue or a salt or ester thereof; Q is hydrogen, thiazolyl, iriazolyl, imidazolyl, benzothiazolyl, or benzoimidazolyl; x is 0, 1, 2, 3, or 4; R¹⁹ is hydrogen, alkyl or aryl; Y¹ is oxygen, sulfur, or nitrogen; Y² is carbon, nitrogen, or oxygen; R²⁰ is hydrogen, alkyl, amino, mercaptoalkyl, alkenyl, alkynyl, cycloalkyl, aryl, arylalkyl, thiazolyl, triazolyl, tetrazolyl, imidazolyl, benzothiazolyl, or benzoimidazolyl; R²¹ is hydrogen, alkyl, mercaptoalkyl, alkenyl, alkynyl, cycloalkyl, aryl, arylalkyl, thiazolyl, triazolyl, tetrazolyl, imidazolyl, benzothiazolyl, benzoimidazolyl, or absent if Y² is oxygen; R²² is hydrogen, alkyl, mercaptoalkyl, alkenyl, alkynyl, cycloalkyl, aryl, arylalkyl, thiazolyl, triazolyl, tetrazolyl, imidazolyl, benzothiazolyl, benzoimidazolyl; or R²² is hydrogen, hydroxyl, alkoxy or aryloxy if Y¹ is nitrogen; or R²² is absent if Y¹ is oxygen or sulfur; or R²² and R²¹ may be linked to form a cyclic moiety if Y¹ is nitrogen; or 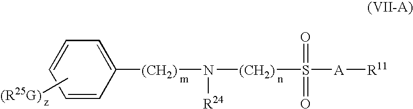
wherein n is 2, 3, or 4; A is oxygen or nitrogen; R¹¹ is hydrogen, salt-forming cation, ester forming group, —(CH₂)ₓ—Q, or when A is nitrogen, A and R¹¹ taken together may be the residue of a natural or unnatural amino acid residue or a salt or ester thereof; Q is hydrogen, thiazolyl, triazolyl, imidazolyl, benzothiazolyl, or benzoimidazolyl; x is 0, 1, 2, 3, or 4; G is a direct bond or oxygen, nitrogen, or sulfur; z is 0, 1, 2, 3, 4, or 5; m is 0 or 1; R²⁴ is selected from a group consisting hydrogen, alkyl, mercaptoalkyl, alkenyl, alkynyl, aroyl, alkylcarbonyl, aminoalkylcarbonyl, cycloalkyl, aryl, arylalkyl, thiazolyl, triazolyl, imidazolyl, benzothiazolyl, and benzoimidazolyl; each R²⁵ is independently selected from hydrogen, halogen, cyano, hydroxyl, alkoxy, thiol, amino, nitro, alkyl, aryl, carbocyclic, or heterocyclic;
and wherein said Protein Aggregation Disorder is not an Amyloid Proteopathy, such that said Protein Azgreiation Disorder is treated or prevented.

2. A method for treating or preventing a Protein Aggregation Disorder in a subject comprising
administering to said subject having a Protein Aggregation Disorder an effective amount of a compound, such that said Protein Aggregation Disorder in said subject is treated or prevented, wherein said compound has one of the following Formulae: 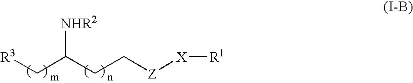
wherein X is oxygen or nitrogen; Z is C═O, S(O)₂, or P(O)OR⁷; m and n are each independently 0, 1, 2, 3, 4, 5, 6, 7, 8, 9 or 10; R¹ and R⁷ are each independently hydrogen, metal ion, alkyl, mercaptoalkyl, alkenyl, alkynyl, cycloalkyl, aryl, a moiety together with X to form natural or unnatural amino acid residue, or —(CH₂)ₚ—Y; Y is hydrogen or a heterocyclic moiety selected from the group consisting of thiazolyl, triazolyl, tetrazolyl, imidazolyl, benzothiazolyl, and benzoimidazolyl; p is 0, 1, 2, 3, or 4; R² is hydrogen, alkyl, mercaptoalkyl, alkenyl, alkynyl, cycloalkyl, aryl, alkylcarbonyl, arylcarbonyl, or alkoxycarbonyl; R³ is hydrogen, amino, cyano, alkyl, mercaptoalkyl, alkenyl, alkynyl, cycloalkyl, heterocyclic, substituted or unsubstituted aryl, heteroaryl, thiazolyl, triazolyl, tetrazolyl, imidazolyl, benzothiazolyl, or benzoimidazolyl; or 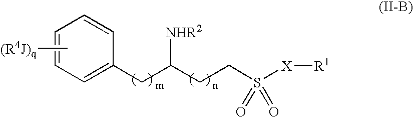
wherein each R⁴ is independently selected from the group consisting of hydrogen, halogen, hydroxyl, thiol, amino, cyano, nitro, alkyl, aryl, carbocyclic or heterocyclic; J is absent, oxygen, nitrogen, sulfur, or a divalent link-moiety consisting of, without limiting to, lower alkylene, alkylenyloxy, alkylenylamino, alkylenylthio, alkylenyloxyalkyl, alkylenylamonialkyl, alkylenylthioalkyl, alkenyl, alkenyloxy, alkenylamino, or alkenylthio; and q is 1, 2, 3, 4, or 5; or 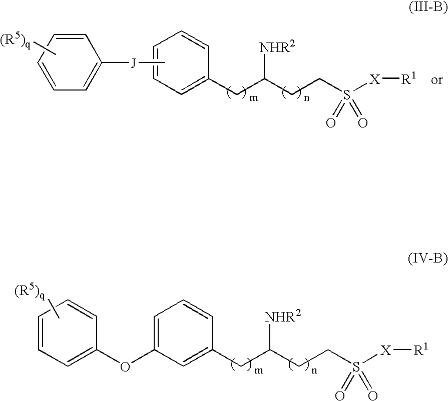
wherein X is oxygen or nitrogen; m and n are each independently 0, 1, 2, 3, 4, 5, 6, 7, 8, 9 or 10; q is 1, 2, 3, 4, or 5; R¹ is hydrogen, metal ion, alkyl, mercaptoalkyl, alkenyl, alkynyl, cycloalkyl, aryl, or a moiety together with X to form a natural or unnatural amino acid residue, or —CH₂)ₚ—Y; Y is hydrogen or a heterocyclic moiety selected from the group consisting of thiazolyl, triazolyl, tetrazolyl, imidazolyl, benzothiazolyl, and benzoimidazolyl; p is 0, 1, 2, 3, or 4; R² is hydrogen, alkyl, mercaptoalkyl, alkenyl, alkynyl, cycloalkyl, aryl, alkylcarbonyl, arylcarbonyl, or alkoxycarbonyl; R⁵ is selected from the group consisting of hydrogen, halogen, amino, nitro, hydroxy, carbonyl, thiol, carboxy, alkyl, alkoxy, alkoxycarbonyl, acyl, alkylamino, acylamino; q is an integer selected from 1 to 5; J is absent, oxygen, nitrogen, sulfur, or a divalent link-moiety consisting of, without limiting to, lower alkylene, alkylenyloxy, alkylenylamino, alkylenylthio, alkylenyloxyalkyl, alkylenylamonialkyl, alkylenylthioalkyl, alkenyl, alkenyloxy, alkenylamino, or alkenylthio; or 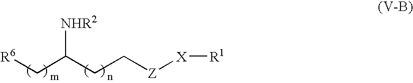
wherein:
R⁶ is a substituted or unsubstituted heterocyclic moiety;
wherein the remaining substituents are as defined above; and wherein said Protein Aggregation Disorder is not an Amyloid Proteopathy.

3. A method for treating or preventing a Protein Aggregation Disorder in a subject comprising
administering to said subject having a Protein Aggregation Disorder an effective amount of a compound, such that said Protein Aggregation Disorder in said subject is treated or prevented, wherein said compound is of the formula 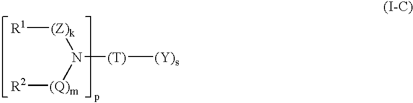
wherein R¹ and R² are each independently a hydrogen atom or a substituted or unsubstituted aliphatic or aryl group; Z and Q are each independently a carbonyl (C═O), thiocarbonyl (C═S), sulfonyl (SO₂), or sulfoxide (S═O) group; k and m are 0 or 1, provided when k is 1, R¹ is not a hydrogen atom, and when m is 1, R² is not a hydrogen atom; T is a linking group and Y is a group of the formula is SO₃⁻X⁺, OSO₃⁻X⁺, or SSO₃⁻x⁺; wherein X⁺ is a cationic group; or
wherein R¹ is an alkyl, alkenyl, or a single-ring aromatic group, where said alkyl group may be substituted with a hydroxyl group; R² is a alkyl, alkenyl, hydroxyalkyl, a single-ring aromatic group, or a hydrogen atom, or R¹ and R², taken together with the nitrogen to which they are attached, form a heterocyclic group which is a fused ring structure; k and m are zero, and p and s are one; T is an alkylene group; Y is SO₃X, and X is a cationic group; or
wherein R¹ is an alkyl, an alkenyl, or an aromatic group; R² is a hydrogen atom, an alkyl group, or an aromatic group, or R¹ and R², taken together, form a heterocyclic group which is a fused ring structure; Z and Q are each independently a carbonyl (C═O), thiocarbonyl (C═S), sulfonyl (SO₂), or sulfoxide (S═O) group; k is 1 and m is 0 or 1, provided when k is 1, R¹ is not a hydrogen atom and when m is 1, R² is not a hydrogen atom; p and s are each 1; T is an alkylene group; and Y is SO₃X, and X is a cationic group; or
wherein R¹ and R² are alkyl, alkenyl, or single-ring aromatic groups, or R¹ and R², taken together with the nitrogen to which they are attached, form a heterocyclic group which is a fused ring structure; k and m are zero, and p and s are one; T is an alkylene group; Y is SO₃X, and X as a cationic group; or
wherein R¹ is an alkyl, alkenyl, or single-ring aromatic group, where said alkyl group may be substituted with a hydroxyl group; R² is a alkyl, alkenyl, single-ring aromatic group, or a hydrogen atom, where said alkyl group may be substituted with a hydroxyl group, or R¹ and R², taken together with the nitrogen to which they are attached, form a heterocyclic group which is a fused ring structure; k and m are zero, and p and s are one; T is an alkylene group; Y is SO₃X, and X is a cationic group;
or pharmaceutically acceptable salts or prodrugs thereof
wherein said Protein Aggregation Disorder is not an Amyloid Proteopathy.

4. A method for modulating a Protein Aggregation Disorder in a subject comprising
administering to said subject having a Protein Aggregation Disorder an effective amount of a compound of claim 1 such that said Protein Aggregation Disorder in said subject is modulated, wherein said Protein Aggregation Disorder is not an Amyloid Proteopathy.

5. A method for modulating detrimental protein aggregation comprising
contacting a detrimental protein aggregate with an effective amount of the compound of claim 1 such that said detrimental protein aggregation is modulated, wherein said detrimental protein aggregate is not associated with an Amyloid Proteopathy.

6. A method for modulating cellular toxicity, comprising
contacting a cell in the presence of a detrimental protein aggregate with an effective amount of a compound of claim 1 such that said cellular toxicity is modulated, wherein said detrimental protein aggregate is not associated with an Amyloid Proteopathy.

7. The method of claim 1, wherein said subject is a mammal.

8. The method of claim 7, wherein said mammal is a human.

9. The method of claim 1, wherein said Protein Aggregation Disorder is selected from the group consisting of Pick's Disease, corticobasal degeneration, progressive supranuclear palsy, amyotrophic lateral sclerosis/parkinsonism dementia complex, Parkinson's Disease (PD), Huntington's disease (HD), dystrophia myotonica, dentatorubro-pallidoluysian atrophy, Friedreich's ataxia, fragile X syndrome, fragile XE mental retardation, spinobulbar muscular atrophy, Wilson's Disease, and spinocerebellar ataxia type 1 (SCA1); spinocerebellar ataxia type 2 (SCA2), Machado-Joseph disease (MJD or SCA3), spinocerebellar ataxia type 6 (SCA6), spinocerebellar ataxia type 7 (SCA7), spinocerebellar ataxia type 17 (SCA 17), chronic liver diseases, cataracts, serpinopathies, haemolytic anemia, cystic fibrosis, neurofibromatosis type 2, demyelinating peripheral neuropathies, retinitis pigmentosa, Marfan syndrome, emphysema, idiopathic pulmonary fibrosis, Argyophilic grain dementia, corticobasal degeneration, diffuse neurofibrillary tangles with calcification, frontotemporal dementia/parkinsonism linked to chromosome 17, Hallervorden-Spatz disease, Nieman-Pick disease type C, and subacute sclerosing panencephalitis.

10. The method of claim 1, wherein said Protein Aggregation Disorder is familial.

11. The method of claim 1, wherein said Protein Aggregation Disorder is idiopathic.

12. The method of claim 1, wherein said Protein Aggregation Disorder is an Alpha-Synucleinopathy.

13. The method of claim 1, wherein said Protein Aggregation Disorder is a Tauopathy provided that said Tauopathy is not Alzheimer's disease, Prion diseases, or cerebral amyloid angiopathy.

14. The method of claim 13, wherein said Tauopathy is selected from the group; Amyotrophic lateral sclerosis/parkinsonism-dementia complex, Argyophilic grain dementia, Corticobasal degeneration, Diffuse neurofibrillary tangles with calcification, Frontotemporal dementia/parkinsonism linked to chromosone 17, Hallervorden-Spatz disease, Multiple system atrophy, Nieman-Pick disease type C, Pick's disease, Progressive supranuclear palsy and Subacute sclerosing panencephalitis.

15. The method of claim 12, wherein the Alpha-Synucleinopathy is Parkinson's Disease, Shy-Drager syndrome, Neurologic orthostatic hypotension, Shy-McGee-Drager syndrome, and Parkinson's plus syndrome.

16. The method of claim 6, wherein, said cellular toxicity is associated with neurotoxicity.

17. The method of claim 6, wherein said cellular toxicity is associated with inclusions.

18. A method for treating or preventing a Neurofibrillary Tangle associated with tau in a subject comprising
administering to said subject having a Neurofibrillary Tangle associated with tau an effective amount of a compound of claim 1, such that said Neurofibrillary Tangle associated with tau in said subject is treated or prevented.

19. A method for modulating a Neurofibrillary Tangle associated with tau in a subject, comprising
administering to said subject having a Neurofibrillary Tangle associated with tau an effective amount of a compound of claim 1, such that said Neurofibrillary Tangle associated with tau in said subject is modulated.

20. A method for treating or preventing an inclusion containing the α-synuclein NAC fragment in a subject comprising
administering to said subject having an inclusion containing the α-synuclein NAC fragment an effective amount of a compound of claim 1, such that said inclusion containing the α-synuclein NAC fragment in said subject is treated or prevented.

21. A method for modulating an inclusion containing the α-synuclein NAC fragment in a subject, comprising
administering to said subject having an inclusion containing the α-synuclein NAC fragment an effective amount of a compound of claim 1, such that said inclusion containing the α-synuclein NAC fragment in said subject is modulated.

22. The method of claims 5, wherein said effective amount is effective to inhibit detrimental protein aggregation.

23. The method of claim 6, wherein said effective amount is effective to inhibit cellular toxicity.

24. The method of claim 1, wherein said method further comprises administering said compound in combination with a pharmaceutically acceptable carrier.

25. A method for modulating detrimental protein aggregation, comprising contacting a protein aggregate with a compound, such that said detrimental protein aggregation is modulated, wherein said compound is according to claim 1.

26. The method of claim 5, wherein said detrimental protein aggregate is extracellular.

27. The method of claim 5, wherein said detrimental protein aggregate is intracellular.

28. The method of claim 5, wherein said detrimental protein aggregate is cytosolic.

29. The method of claim 5, wherein said detrimental protein aggregate is nuclear.

30. The method of claim 5, wherein said detrimental protein aggregate is intra-membranal.

31. The method of claim 5, wherein said detrimental protein aggregate is in the endoplasmic reticulum.

32. The method of claim 5, wherein said detrimental protein aggregate is in the trans-Golgi network.

33. The method of claim 5, wherein said detrimental protein aggregate is associated with an aggresome.

34. The method of claim 5, wherein said detrimental protein aggregate is associated with misfolding of mature protein.

35. The method of claim 5, wherein said detrimental protein aggregate is associated with improper degradation of protein.

36. The method of claim 5, wherein said detrimental protein aggregate is associated with a misfolded protein that has evaded the ubiquitin-proteasome system.

37. The method of claim 5, wherein said detrimental protein aggregation is inhibited.

38. The method of claim 5, wherein said detrimental protein aggregation is modulated by enhancing degradation of said protein aggregate.

39. The method of claim 5, wherein said detrimental protein aggregation is modulated by increasing the clearance of said protein aggregate.

40. The method of claim 5, wherein said detrimental protein aggregation is associated with fibrils, β-sheets, or hydrophobic domains.

41. A pharmaceutical composition, comprising an effective amount of a compound of claim 1, wherein said effective amount is effective to treat a Protein Aggregation Disorder and a pharmaceutically acceptable carrier.

42. The pharmaceutical composition of claim 41, wherein, said Protein Aggregation Disorder is selected from the group consisting of Pick's Disease, corticobasal degeneration, progressive supranuclear palsy, amyotrophic lateral sclerosis/parkinsonism dementia complex, Parkinson's Disease (PD), Huntington's Disease (HD), dystrophia myotonica, dentatorubro-pallidoluysian atrophy, Friedreich's ataxia, fragile X syndrome, fragile XE mental retardation, spinobulbar muscular atrophy, Wilson's Disease, and spinocerebellar ataxia type 1 (SCA1) gene; spinocerebellar ataxia type 2 (SCA2), Machado-Joseph disease (MJD or SCA3), spinocerebellar ataxia type 6 (SCA6), spinocerebellar ataxia type 7 (SCA7) caused, spinocerebellar ataxia type 17 (SCA17), chronic liver diseases, cataracts, serpinopathies, haemolytic anemia, and cystic fibrosis. Pick's Disease, corticobasal degeneration, progressive supranuclear palsy, amyotrophic lateral sclerosis/parkinsonism dementia complex, Parkinson's Disease (PD, Huntington's disease (HD), dystrophia myotonica, dentatorubro-pallidoluysian atrophy, Friedreich's ataxia, fragile X syndrome, fragile XE mental retardation, Machado-Joseph disease, spinobulbar muscular atrophy, Wilson's Disease, spinocerebellar ataxia, and cataracts.

43. A packaged composition for treatment of a Protein Aggregation Disorder, comprising a compound of claim 1 and directions for using said compound for treating said Protein Aggregation Disorder.

44. A method for identifying a candidate compound useful for treatment or prevention of a Protein Aggregation Disorder comprising:
a) administering a test compound to a mouse model of a Protein Aggregation Disorder;
b) determining the effectiveness of said test compound to prevent, modulate, reduce or inhibit the development of progressive degenerative changes associated with said Protein Aggregation Disorder in said mouse model;
c) identifying said selected compound as a candidate compound useful as a treatment or prevention for a Protein Aggregation Disorder.

45. The method of claim 5, wherein said detrimental protein aggregate is associated with at least one of the proteins selected from the group consisting of: α-synuclein, tau, NAC, huntingtin, DRPLA, Schwannomin, cytokeratin, myelin protein 22, rhodopsin, atrophin-1, fibrillin-1, ataxin-1, ataxin-2, ataxin-3, ataxin-6, ataxin-7, ataxin-17, androgen receptor, surfactant protein-C, and alphal-antitrypsin.

46. A method for identifying a candidate compound useful preventing, modulating, reducing or inhibiting detrimental protein aggregates comprising:
a) contacting a detrimental protein aggregate in vitro;
b) determining the effectiveness of said test compound to prevent, modulate, reduce or inhibit detrimental protein aggregates;
c) identifying said selected compound as a candidate compound useful as a treatment or prevention for a Protein Aggregation Disorder.

47. The method of claim 6, wherein said cell is a neuronal cell or a glial cell.

48. The method of claim 44, further comprising determining the effectiveness of said test compound to facilitate clearance of said detrimental protein aggregates.

49. The method of claim 44, further comprising determining the effectiveness of said test compound to facilitate the degredation of said detrimental protein aggregates.

50. A method for modulating detrimental protein aggregation comprising
contacting a detrimental protein aggregate with an effective amount of the compound of claim 1 such that clearance of said detrimental protein aggregation is modulated, thereby modulating detrimental protein aggregation, wherein said detrimental protein aggregate is not associated with an Amyloid Proteopathy.

51. A method for modulating detrimental protein aggregation comprising
contacting a detrimental protein aggregate with an effective amount of the compound of claim 1 such that cellular toxicity of said detrimental protein aggregation is modulated, thereby modulating detrimental protein aggregation, wherein said detrimental protein aggregate is not associated with an Amyloid Proteopathy.

52. A method for modulating detrimental protein aggregation comprising
contacting a protein that has a propensity to form β-sheet structures with an effective amount of the compound of claim 1 such that said detrimental protein aggregation is modulated, wherein said Protein Aggregation Disorder is not an Amyloid Proteopathy.

53. A method for modulating detrimental protein aggregation comprising
contacting a protein that has a propensity to form β-sheet structures with an effective amount of the compound of claim 1 such that clearance of said detrimental protein aggregation is modulated, thereby modulating detrimental protein aggregation, wherein said Protein Aggregation Disorder is not an Amyloid Proteopathy.

54. A method for modulating detrimental protein aggregation comprising
contacting a protein that has a propensity to form β-sheet structures with an effective amount of the compound of claim 1 such that cellular toxicity of said detrimental protein aggregation is modulated, thereby modulating detrimental protein aggregation, wherein said Protein Aggregation Disorder is not an Amyloid Proteopathy.

55. The method of claim 1, wherein said carrier molecule is selected from the group consisting of carbohydrates, polymers, peptides, peptide derivatives, aliphatic groups, alicyclic groups, heterocyclic groups, aromatic groups and combinations thereof.
